(19) United States
(12) Patent Application Publication
(10) Pub. No.: US 2021/0145795 A1
(43) Pub. Date: May 20, 2021

(54) SOCE FACILITATORS FOR USE IN TREATING OR PREVENTING VIRAL INFECTIONS

(71) Applicant: THE UNIVERSITY OF NOTTINGHAM, Nottingham (GB)

(72) Inventors: Kin-Chow CHANG, Sutton Bonington (GB); Leah Victoria GOULDING, Sutton Bonington (GB)

(21) Appl. No.: 17/045,083

(22) PCT Filed: Apr. 4, 2019
(86) PCT No.: PCT/GB2019/050977

§ 371 Date: Oct. 2, 2020

(30) Foreign Application Priority Data
Apr. 5, 2018 (GB) 1805665.5

(51) Int. Cl. A61K 31/366; A61K 31/015; A61K 31/19; A61K 45/06; A61K 9/00; G01N 33/569

(52) U.S. Cl. CPC A61K 31/366; A61K 31/015; A61K 31/19; G01N 2333/115; A61K 9/0078; G01N 33/56983; G01N 2333/11; A61K 45/06

(57) ABSTRACT

The invention provides an agent for the treatment or prevention of viral infection in a subject. The agent is preferably a compound of Formula (I)

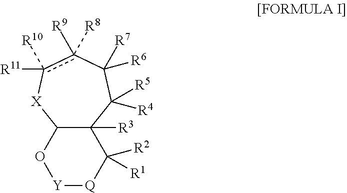

wherein R¹-R¹¹, X, Y and Q are as defined herein. Also provided are pharmaceutical compositions and combinations comprising such agents. An in vitro method of evaluating the antiviral activity or potential antiviral activity of a compound against a virus is also provided.

### FIELD OF THE INVENTION

The present invention relates to compounds and their use in the treatment or prevention of viral infection in a subject. The invention also provides pharmaceutical compositions comprising such compounds. The compounds can be used in combination therapy, for example with one or more additional antiviral agents. The invention also provides an aerosol formulation comprising such compounds. The compounds find use in treatment of viral infections, particularly infection by RNA viruses such as influenza viruses and Paramyxoviruses. The invention also provides an in vitro method of evaluating antiviral activity of a compound against a virus such as an RNA virus.

### BACKGROUND

Influenza A virus is a major global pathogen of humans and a wide range of mammals and birds. One particular challenge to treating influenza virus arises from the high mutational rate of the virus, which occurs through re-assortment of the segmented genome between different virus strains and as a result of its error-prone RNA polymerase complex. The arising high mutational rate of the virus presents serious challenges to the development of effective antiviral drugs and vaccines.

A number of approaches have been proposed for targeting influenza infections. One strategy that has been considered relies on inhibition of the M2 proton channel present in the viral envelope of the influenza A virus. Inhibition of the M2 channel prevents viral uncoating with the result that the ribonucleoprotein complex core fails to promote infection. Pharmaceuticals targeting the M2 channel include amantadine and rimantadine. However, the build up of viral resistance against these compounds has led to the need for improved pharmaceuticals to target the virus.

A second strategy that has been considered relies on inhibition of the influenza neuraminidase enzyme which is responsible for cleaving glycosidic linkages of neuraminic acids. Anti-neuraminidases are the only group of drugs recommended by the World Health Organisation. Known anti-neuraminidases include zanamivir, oseltamivir, laninamivir and peramivir. These drugs function by blocking the function of viral neuraminidases, ultimately preventing virus release by budding from the host cell membrane. However, the efficacy of these drugs has been called into question. Furthermore, the potential for these drugs to combat virulent strains of viral infection such as epidemic and pandemic influenza strains is limited as such compounds in their widespread use are highly vulnerable to the development of virus resistance. There is thus a pressing need for new classes of pharmaceutical which target viral infection but which are less vulnerable to development of viral resistance.

Recognising this need, attempts have been previously made to target the viral host, rather than the virus per se. These approaches typically rely on sub-type specific vaccines against particular viral infections. This approach is widely considered to be effective and safe. However, the process of identifying and generating appropriate vaccines is too protracted to allow a rapid response to highly infective viral strains, such as in the case of pandemic influenza. In addition, viral response to such vaccines is complex and can be unpredictable. For example, it has previously been shown that antibodies generated in vivo following vaccination against a particular viral strain can in some circumstances have unexpected and adverse effects in terms of increasing the infectivity of other viral strains. Moreover, adequate broad spectrum vaccines have not yet been developed, thus requiring yearly antigen updates.

Accordingly, there is an urgent need for new approaches to targeting viral infection in a subject. In particular, there is a need for new therapies for viral infection which have reduced vulnerability to development of viral resistance, which have wide applicability thus avoiding the need for lengthy vaccine generation, thus offering a viable protection against virulent viral strains, and/or which have predictable effects and which avoid enhancing infectivity of off-target viral strains. The present invention aims to address some or all of these issues.

### SUMMARY OF THE INVENTION

The present inventors have surprisingly found that the brief cellular activation of a host Ca²⁺ signalling pathway, Ca²⁺ release-activated Ca²⁺ (CRAC) entry, induces a potent and sustained antiviral host innate immune response. The inventors have surprisingly found that CRAC entry operated store operated Ca²⁺ entry (SOCE) induces prolonged host resistance that dramatically reduces virus production. This antiviral response is functionally effective pre- or post-infection in a variety of cell types. The inventors further found that certain compounds described herein are particularly suited to activating SOCE. The inventors thus recognised that such compounds have applications in treating and/or preventing viral infection in a subject. The compounds can be advantageously provided in the form of an aerosol formulation. The compounds can advantageously be used in the form of a pharmaceutical composition comprising a pharmaceutically acceptable carrier or diluent. The compounds can also be advantageously used in the form of a combination comprising an additional antiviral agent. Such combination therapies have particular relevance in the prevention or treatment of viral infection caused by highly infectious viral strains such as epidemic or pandemic influenza strains and Paramyxoviridae viruses.

Ca²⁺ Release-Activated Ca²⁺ (CRAG) Entry, Innate Immunity and Endoplasmic Stress

Ca²⁺ release-activated Ca²⁺ (CRAC) entry is a primary process for Ca²⁺-specific signalling and for maintenance of intracellular Ca²⁺ concentration. The process of CRAC entry begins with Ca²⁺ depletion from the endoplasmic reticulum (ER) Ca²⁺ store which is primarily triggered by inositol 1,4,5-trisphosphate [IP₃]) produced by activated phospholipase C (PLC). Binding of IP₃ to its ER IP₃ receptor leads to Ca²⁺ release into the cytosol, hence the term “ER Ca²⁺ store depletion”.

Upon detection of reduced Ca²⁺ within the ER lumen by stromal interaction molecules (STIM1 and STIM2), these Ca²⁺ sensors undergo conformational change and migrate to sites in the ER membrane in close proximity to the plasma membrane. Here they interact with plasma membrane-sited store-operated Ca²⁺ (SOC) channel proteins (Orai1, Orai2 and Orai3) to form fully activated tetrameric SOC channels leading to extracellular Ca²⁺ entry known as store operated Ca²⁺ entry (SOCE). SOCE refers to the activated function of the STIM-Orai complex in directing extracellular Ca²⁺ influx.

CRAC entry is evident in many types of immune and non-immune cells, and contributes to the control of a variety of physiological functions. Some studies have sought to investigate the role of SOCE in innate immunity. However, the role of CRAC (if any) in innate immunity remains unclear. It is known, however, that excessive extracellular Ca²⁺ influx is detrimental and promotes apoptosis (Ueda et al., 2010; Flourakis et al., 2010). The antiviral effects of SOCE (in particular the effect of SOCE on influenza virus) are not known.

Ca²⁺ depletion from the ER Ca²⁺ store is often accompanied by ER stress-associated accumulation of unfolded or misfolded proteins in the ER which in turn triggers the unfolded protein response (UPR), a cellular adaptive response that attempts to restore homeostasis in protein production. UPR typically involves activation of three ER-transmembrane receptors (ER stress sensors): protein kinase RNA-like endoplasmic reticulum kinase (PERK), activating transcription factor-6 (ATF6) and inositol-requiring enzyme (kinase) 1 (IRE1α). Activated PERK, a serine threonine kinase, phosphorylates and inactivates the eIF2α subunit within minutes to hours of UPR activation to attenuate global protein synthesis including that of influenza virus (Janssens et al., 2014; Silva et al., 2007; Landera-Bueno et al., 2017). Stimulation of ATF6 and PERK can lead to the activation of NF-κB and induction of cytokines (Janssens et al., 2014). IRE1α is a major contributor to chronic inflammatory conditions; it recruits NOD1/2-TRAF2-RIPK2 complex leading to the activation of NF-κB that induces IL6 expression (Keestra-Gounder et al., 2016). By disrupting viral protein synthesis via PERK activation (Landera-Bueno et al., 2017), or possibly by stimulating RIG-I-type I IFN cascade via IRE1α activation (Lencer et al., 2015; Cho et al., 2013; Perry et al., 2012), ER stress could conceivably play a role in limiting virus replication. As with SOCE, the role of ER stress, in particular the function of IRE1α receptor, as an antiviral mediator of influenza virus replication is unclear.

Roles of Ca²⁺ and CRAG Entry in Virus Replication

Ca²⁺ signalling, from extracellular influx of activated ion channels or from indirect signal transduction that leads to Ca²⁺ release from ER store, affects a whole host of cellular processes including excitation-contraction, motility, exocytosis and apoptosis. Modulation of Ca²⁺ signalling is also a key step in the pathogenesis of a number of viruses. Raised cytosolic Ca²⁺ or the process of extracellular Ca²⁺ entry is actively triggered by different viruses to facilitate their replication or pathogenesis.

For example, in rotavirus infection, increase in cytosolic Ca²⁺, mediated by viral non-structural protein 4 (NSP4), has been shown to be necessary for virus replication (Hyser et al., 2013). Omission of Ca²⁺ from culture media inhibits rotavirus production (Michelangeli et al., 1995). NSP4, a transmembrane ER bound viroporin, induces chronic ER Ca²⁺ store depletion throughout infection leading to sustained extracellular Ca²⁺ influx through activating the STIM-Orai channel complex (SOCE) (Hyser et al., 2013), and other Ca²⁺ entry channels such as transient receptor potential cation channels (TRPCs) (Díaz et al., 2012).

Hepatitis B virus X protein (HBx) raises cytosolic Ca²⁺ through activation of SOC channels to enhance HBV replication in primary rat hepatocytes (Casciano et al., 2017). HBx does not appear to actively elicit ER Ca²⁺ store depletion but promotes mitochondrial Ca²⁺ uptake (Yang and Bouchard, 2012), and does not increase the expression of STIM1 or Orai1 (Casciano et al., 2017). Similarly, Epstein Barr virus (EBV) latent membrane protein-1 (LMP1) has been shown to increase Ca²⁺ influx through SOC channels (Orai1) and to raise Orai1 expression in B lymphoid cells in connection with its oncogenic function. Like HBx, ER Ca²⁺ store appears not to be depleted (Dellis et al., 2011).

SOCE triggered by matrix proteins of hemorrhagic fever viruses at the late stages of viral replication has been shown to be necessary for virus budding (Han et al., 2015).

Accordingly, whilst the precise role of Ca²⁺ influx and CRAC in particular is complex, studies to date indicate that as a general rule promoted extracellular Ca²⁺ entry is proviral. This finding has also been demonstrated in influenza A virus infection. For influenza A, Ca²⁺ influx has been identified as a pro-viral factor that is required for cell entry (via phosphatidylinositol 4-phosphate 5-kinase [PIP5K] clathrin-mediated, and Ras-mediated clathrin-independent endocytosis) and replication (Fujioka et al., 2013).

In studying ER Ca²⁺ store depletion, use has been made of the compound thapsigargin (TG). TG is a known inhibitor of sarcoplasmic/endoplasmic reticulum Ca²⁺-ATPase (SERCA) pump, which impedes the replenishment of Ca²⁺ in the ER store. The role of TG in viral replication has not been extensively studied. However, a previous study reported that MDCK (Madin-Darby Canine Kidney) cells treated with a low dose of TG (0.001 μM) over 48 h of infection by influenza A virus was found to increase progeny influenza virus output, suggesting that Ca²⁺ released from the ER supports influenza virus replication (Fujioka et al., 2013). The indication from this study is that prolonged exposure of TG has a proviral effect in influenza infection.

Other studies have sought to probe the pro-viral function of Ca²⁺. One hypothesis that has been advanced is that Ca²⁺ could physiologically switch on the sialidase activity of newly synthesised influenza virions facilitating their release from host cell membrane (Chong et al., 1991). Furthermore, Ca²⁺/calmodulin-dependent protein kinase (CaM kinase) IIb (CAMK2B) has been implicated in promoting influenza viral RNA transcription (Konig et al., 2010). Accordingly, Ca²⁺ appears to promote the replication of a number of viruses including influenza A virus (Zhou et al., 2009; Fujioka et al., 2013; Marois et al., 2014).

Thus, in summary, modulation of Ca²⁺ signalling is a key step in the pathogenesis of a number of viruses. Raised cytosolic Ca²⁺ levels and the process of extracellular Ca²⁺ entry is actively triggered by different viruses to facilitate their replication or pathogenesis. In this context, the present invention can be readily understood.

The Present Invention

The present inventors sought to elucidate the role of CRAC entry in influenza A virus replication. The present inventors surprisingly found that, contrary to expectation, CRAC influx, for example activated by brief TG exposure at non-toxic doses, induced prolonged host resistance that dramatically reduced influenza A virus production. The inventors surprisingly found that this antiviral response is functionally effective in a variety of cell types, including human primary respiratory epithelial cells, the frontline cell type in the initiation of influenza virus infection in vivo. Furthermore, the antiviral response was effective when activated before or during virus infection. These results are described in more detail herein.

Accordingly, the invention provides a Store-Operated Ca²⁺ Entry (SOCE) facilitator for use in the treatment or prevention of viral infection in a subject. Preferably, the SOCE facilitator is as further described herein, such as for example a compound of Formula (I) or a pharmaceutically acceptable salt, derivative, or prodrug thereof.

The invention also provides a compound for use in treating viral infection in a subject in need thereof, wherein said compound is a sesquiterpene or sesquiterpene lactone. The sesquiterpene or sesquiterpene lactone is preferably a compound of Formula (I) or a pharmaceutically acceptable salt, derivative, or prodrug thereof. The invention also provides a pharmaceutical composition for use in the treatment or prevention of viral infection in a subject comprising an SOCE facilitator as described herein or a compound which is a sesquiterpene or sesquiterpene lactone, and at least one pharmaceutically acceptable carrier or diluent. Also provided is a combination comprising (i) an SOCE facilitator as described herein or a compound which is a sesquiterpene or sesquiterpene lactone and (ii) an additional antiviral agent, and optionally (iii) at least one pharmaceutically acceptable carrier or diluent. Further provided is the use of such a combination in the treatment or prevention of viral infection in a subject. The invention also provides an aerosol formulation of an SOCE facilitator or a compound which is a sesquiterpene or sesquiterpene lactone as described herein.

The invention thus also provides an SOCE facilitator, a compound, composition and/or a combination as described herein for use in the treatment or prevention of viral infection in a subject in need thereof. Also provided is a method for treating or preventing viral infection in a subject, wherein said method comprises administering to said subject an effective amount of an SOCE facilitator, a compound, a composition and/or a combination as described herein. Further provided is the use of an SOCE facilitator, a compound, a composition and/or a combination as described herein in the manufacture of a medicament for use in treating or preventing viral infection in a subject.

The invention also provides an in vitro method of evaluating the antiviral or potential antiviral activity of a compound against a virus. The method comprises assessing the ability of the compound to activate CRAC entry mediated SOCE.

The antiviral properties of Store-Operated Ca²⁺ Entry (SOCE) facilitators (such as the sesquiterpene or sesquiterpene lactone compounds described herein) administered at non-toxic dosages is a surprising finding of the present invention. Furthermore, even transient administration has been shown to lead to a sustained antiviral response. Studies that have previously claimed to observe antiviral effects of SOCE facilitators such as thapsigargin have typically used concentrations of such compounds in the toxic range, have linked the alleged antiviral effects with increased cytotoxicity, and have shown that the SOCE facilitator leads to increased cell death compared to viral infection alone. This is contrary to the present invention, which is based in part on the finding that brief exposure to SOCE facilitators at non-cytotoxic doses leads to CRAC influx and induces prolonged host resistance that dramatically reduces viral production.

### DESCRIPTION OF THE FIGURES

FIG. 1—Raised extracellular Ca²⁺ reduced influenza virus output from porcine primary muscle (myotube) and neonatal pig tracheal epithelial (NPTr) cells. FIGS. 1A and B show that raising extracellular [Ca²⁺] in the culture media of influenza virus infected cells (NPTr cells (FIG. 1A) and primary porcine muscle cells, myotubes (FIG. 1B) resulted in significantly reduced production of progeny virus. Significance is in relation to corresponding cells cultured in the presence of 100mg/L Ca²⁺. FIGS. 1C and D show that the different Ca²⁺ concentrations on corresponding uninfected cells had no adverse impact on cell viability. Results are described in Example 2.

FIG. 2—Ca²⁺ influx triggered by thapsigargin (TG) was dependent on extracellular Ca²⁺.

FIG. 2A: Intracellular Ca²⁺ accumulation in porcine myoblasts, NPTr cells, PTECs and NHBE cells exposed to TG for 10 min at indicated non-toxic concentrations in the presence of 0 mg/L (black) or 200 mg/L (grey) extracellular calcium chloride. FIG. 2B: Porcine myoblasts, NPTr cells, PTECs and NHBE cells were infected with USSR H1N1 virus at 2.0 MOI, 1.0 MOI, 1.0 MOI and 1.0 MOI respectively for 15 min before intracellular Ca²⁺ fluorescence readings were taken. Results are described in Example 3.

FIG. 3—TG priming of NPTr cells, myoblasts and NHBE cells reduced progeny virus output. Influenza virus output (USSR H1N1 or pdm H1N1 virus), normalised viral M-gene expression and cell viability of NPTr cells (FIG. 3A), myoblasts (FIG. 3B) and NHBE cells (FIG. 3C) are shown at the indicated non-toxic TG concentrations. Results are described in Example 3.

FIG. 4—TG-primed NPTr cells, PTECs and porcine myoblasts showed sustained potency in reducing influenza virus production. The antiviral state of TG-primed NPTr cells (FIG. 4A), PTECs (FIG. 4B) and porcine myoblasts (FIG. 4C) lasted for at least 24 h post-TG exposure. Influenza virus output (USSR H1N1 virus) and normalised viral M-gene expression of NPTr cells, myoblasts and NHBE cells are shown. Results are described in Example 4.

FIG. 5—Cells primed with TG before or during infection were comparably effective in inhibiting virus production. Indicated influenza virus output, normalised viral M-gene expression, and expression of type I IFN associated genes (RIG-I and OAS1) are shown for NPTr cells (FIG. 5A), NHBE cells (FIG. 5B) and myoblasts (FIG. 5C). Results are described in Examples 4 and 5.

FIG. 6—NPTr cells (FIG. 6A), porcine myoblasts (FIG. 6B) and NHBE cells (FIG. 6C) primed with TG showed elevated expression of type I IFN associated genes (RIG-I and OAS1) in response to infection by USSR H1N1 virus. Significance is in relation to corresponding DMSO control. Results are described in Example 5.

FIG. 7—NPTr cells (FIG. 7A), porcine myoblasts (FIG. 7B) and NHBE cells (FIG. 7C) primed with TG showed no reduction in viral NP and M1 proteins after 24 h of USSR H1N1 virus infection. Significance is in relation to corresponding DMSO control. Results are described in Example 6.

FIG. 8—Pre-treatment with TG did not appear to affect the morphology of budding influenza virions from infected NPTr cells. NPTr cells were grown on Thermanox coverslips and pre-treated with DMSO (FIGS. 8A and B) or 0.5 μM TG (FIG. 8C and D) for 30 min, subsequently washed with PBS and infected with USSR H1N1 at 1.0 MOI for 12 h. NPTr cells were processed for transmission electron microscopy. Examples of influenza virions marked with arrowheads. Scale bars a, c=500 nm, b,d=1000 nm. Results are described in Example 6.

FIG. 9—Priming with non-toxic doses of TG induced ER stress in a dose dependent response in NPTr cells (FIG. 9A), porcine myoblasts (FIG. 9B) and NHBE cells (FIG. 9C) primed with TG and infected with USSR H1N1 virus. Significance is in relation to corresponding DMSO control. Results are described in Example 7.

FIG. 10—Tunicamycin, at non-toxic dose doses did not induce Ca²⁺ influx in NPTr cells but strongly up-regulated expression of ER stress genes (FIG. 10A), had only a limited effect in reducing virus production as compared to TG (FIG. 10B), and had little or no effect on the expression of type I IFN associated genes (RIG-I, OAS1 and PKR) (FIG. 10C). Significance is in relation to corresponding DMSO control. Results are described in Example 8.

FIG. 11—Over-expression of SOCE members (STIM1 and Orai isoforms) reduced virus production to a similar extent as TG priming (FIGS. 11Ai and Aii) without affecting viral M protein and NP production (FIGS. 11Aiii and 11Aiv), did not appear to increase expression of type I IFN associated genes (RIG-I and OAS1) (FIG. 11B), and had little or no effect on the expression of ER stress associated genes (FIG. 11C) in NPTr cells, transiently transfected with the indicated plasmids and infected with USSR H1N1. Significance is in relation to corresponding control. Results are described in Example 9.

FIG. 12—Over-expression of SOCE members (STIM1 and Orai isoforms) reduced virus production to a similar extent as TG priming (FIGS. 12Ai and Aii) without affecting viral M protein and NP production (FIGS. 12Aiii and Aiv), had little or no effect on expression of type I IFN associated genes (FIG. 12B) and ER stress associated genes (FIG. 12C) in porcine myoblasts, transiently transfected with the indicated plasmids and infected with USSR H1N1. Significance is in relation to corresponding control. Results are described in Example 9.

FIG. 13—Individual over-expression of CRAC2RA and STIMATE, positive regulators of SOCE, in NPTr cells significantly reduced progeny USSR H1N1 and pdm H1N1 virus release (FIG. 13Ai) without reduction in viral M gene expression (FIG. 13Aii), showed reduction in expression of type I IFN associated genes (RIG-I and OAS1) in response to USSR H1N1 virus infection (FIG. 13B), and resulted in little increase in the expression of ER stress associated genes (DDIT3, HSPA5 and HSP90B1) in uninfected cells (FIG. 13C). Significance is in relation to corresponding control. Results are described in Example 9.

FIG. 14—Individual over-expression of CRAC2RA and STIMATE in porcine myoblasts significantly reduced progeny influenza virus release (FIG. 14Ai) with variable effects on viral M-gene expression (FIG. 14Aii), showed little or no effect on expression of type I IFN associated genes (RIG-I and OAS1) in response to USSR H1N1 virus infection (FIG. 14B), and had little or no effect on the expression of ER stress associated genes (DDIT3, HSPA5 and HSP90B1) in uninfected cells (FIG. 14C). Significance is in relation to corresponding control. Results are described in Example 9.

FIG. 15—Individual over-expression of CRAC2RA and STIMATE in NHBE cells significantly reduced progeny USSR H1N1 virus release without reduction in viral M gene expression (FIG. 15A), had little effect on expression of type I IFN associated genes (RIG-I and OAS1) in response to infection (FIG. 15B), and had little or no effect on the expression of ER stress associated genes (DDIT3, HSPA5 and HSP90B1) in uninfected cells (FIG. 15C). Significance is in relation to corresponding control. Results are described in Example 9.

FIG. 16—Inhibition of SOCE increased influenza virus output. Exposure of NPTr cells to Orai inhibitors, 150 nM BTP2 and 5 μM Synta66, conferred small rise in progeny virus output (FIG. 16A). Progeny virus output raised in STIM1-knockdown NHBE cells (FIG. 16B). Knockdown of STIM1 but not Orai1 in NHBE cells reduced the inhibitory effect of TG in virus production (FIG. 16C). Expression of type I IFN associated genes was reduced in STIM1 and Orai 1 knockdown NHBE cells in response to infection (FIG. 16D). Significance is in relation to corresponding control. Results are described in Example 10.

FIG. 17—Priming of NPTr cells with non-toxic doses of CPA (<5 μM) (FIG. 17A) did not induce extracellular Ca²⁺ influx (FIG. 17B), had no effect on progeny virus output (USSR H1N1 virus [FIG. 17C] and pdm H1N1 virus [FIG. 17D]). Significance is in relation to corresponding DMSO control. Results are described in Example 11.

FIG. 18—Schematic summary of how TG mediated-CRAC entry may resist influenza virus production.

FIG. 19—Priming of NPTr cells with artemisinin, a compound structurally related to TG, reduced progeny USSR H1N1 and pdm H1N1 virus release (FIG. 19A). Effect of artemisinin on extracellular Ca²⁺ influx in NPTr cells, PTECs and myoblasts is shown in FIG. 19B. Significance is in relation to corresponding DMSO control. Results are described in Example 12.

FIG. 20—Separate priming of NPTr cells with additionally indicated sesquiterpenes (valerenic acid, (+)-ledene, dihydroleucodine and artemisinin, in particular dehydroleucodine and (+)-ledene, for 30 min prior to infection reduced USSR H1N1 virus production like that of TG (FIG. 20A to C). Priming with selected sesquiterpenes did not adversely affect cell viability (FIG. 20D). The effect of priming with selected sesquiterpenes on extracellular Ca²⁺ influx is shown in FIG. 20E. Significance is in relation to corresponding DMSO control. Results are described in Example 13.

FIG. 21—Separate priming of NHBE cells with (+)-ledene and dehydroleucodine at 2.5 μM for 30 min prior to infection with USSR H1N1 reduced progeny virus output (FIG. 21A). Sesquiterpenes used at 2.5 μM were non-toxic to cells (FIG. 21B). Further comparison of compounds priming of NHBE cells in reducing USSR H1N1 virus production (FIG. 21C). Significance is in relation to corresponding DMSO control. Results are described in Example 13.

FIG. 22—TG priming of HEp2 cells at non-toxic doses blocks RSV production. HEp2 cells were incubated with indicated concentrations of TG or control DMSO. TG at non toxic levels (FIG. 22B) blocks RSV production (FIG. 22A). FIG. 22C shows representative immuno-staining results. Results are described in Example 14.

FIG. 23—TG-activated anti-RSV state in HEp2 cells lasts more than 48 h and is rapidly triggered during infection. FIG. 23A: HEp2 cells were pre-incubated with indicated concentrations of TG or control DMSO for 30 min, rinsed and further cultured for 24 or 48 h in normal media followed by RSV infection at 0.1 MOI for 3 days. FIG. 23B: HEp2 cells were infected with RSV at 0.1 MOI for 24 or 48h followed by priming with TG at indicated concentrations or DMSO control for 30 min. Fresh media were used to replace TG containing media of 24 h infected cells; supernatants collected earlier from 48 h infected cells were used to replace TG containing media of 48 h infected cells. TG effectively blocks the RSV production when administered either prior to infection (FIG. 23A) or during infection (FIG. 23B). Results are described in Example 15.

### DETAILED DESCRIPTION OF THE INVENTION

Definitions

As used herein, a C₁₋₇ alkyl group is a linear or branched alkyl group containing from 1 to 7 carbon atoms. A C₇ alkyl group may be n-heptyl. A C₁₋₇ alkyl group is often a C₂₋₄ alkyl group. Examples of C₁₋₄ alkyl groups include methyl, ethyl, n-propyl, iso-propyl, n-butyl, iso-butyl, sec-butyl, and tert-butyl. A C₁₋₄ alkyl group is often a C₁₋₂ alkyl group or a C₂₋₄ alkyl group. A C₁ to C₂ alkyl group is methyl or ethyl, typically methyl. A C₂₋₄ is often an n-propyl group. For the avoidance of doubt, where two alkyl groups are present, the alkyl groups may be the same or different.

As used herein, a C₂₋₇ alkenyl group is a linear or branched alkenyl group containing from 2 to 7 carbon atoms and having one or more, e.g. one or two, typically one double bonds. Typically a C₂₋₇ alkenyl group is a C₃₋₅ alkenyl group. Examples of C₃₋₅ alkenyl groups include propenyl, butenyl and pentenyl. A C₃₋₅ alkenyl group is typically a C₄ alkyenyl group such as n-butenyl or but-2-en-2-yl; typically but-2-en-2-yl (—C₄H₇). For the avoidance of doubt, where two alkenyl groups are present, the alkenyl groups may be the same or different.

An alkyl or alkenyl group as used herein may be unsubstituted or substituted. Unless otherwise stated, substituted alkyl, alkenyl or alkynyl groups typically carry one or more, e.g. 1, 2, 3 or 4, such as one, two or three e.g. one, or two, e.g. one substituent selected from —COOH, —C₆H₄—COOH, halogen, —OH and the like; typically the substituent is selected from —COOH and —C₆H₄—COOH. The substituents on a substituted alkyl, alkenyl or alkynyl group are typically themselves unsubstituted unless otherwise stated. Where more than one substituent is present, these may be the same or different.

As used herein, a halogen is typically chlorine, fluorine, bromine or iodine and is preferably chlorine, bromine or fluorine, especially chorine or fluorine, especially fluorine.

As used herein, an oxo group is an oxygen atom bonded by a double bond to carbon or another element; for example an oxo group may be bonded to carbon to form a >C═O moiety.

A 5-membered carbocyclic group is a cyclic hydrocarbon containing 5 carbon atoms. A 6-membered carbocyclic group is a cyclic hydrocarbon containing 6 carbon atoms. A carbocyclic group may be saturated or partially unsaturated. A 5-membered partially unsaturated carbocyclic group is a cyclic hydrocarbon containing 5 carbon atoms and containing 1 or 2, e.g. 1 double bond. A 6-membered partially unsaturated carbocyclic group is a cyclic hydrocarbon containing 6 carbon atoms and containing 1 or 2, e.g. 1 double bond. Examples of 5- and 6-membered carbocyclic groups include cyclopentyl, cyclopentenyl, and cyclohexyl groups. A 5- or 6-membered carbocyclic group can be fused to another group such as a further cyclic group to form a fused ring compound.

As used herein, a fused ring compound is a compound comprising two cyclic moieties sharing a common bond between two atoms.

A carbocyclic group may be unsubstituted or substituted as described herein. For example, a carbocyclic group may be unsubstituted or substituted with 1, 2, 3, 4 or 5, typically 1, 2, 3 or 4 such as e.g. 1, 2 or 3 substituents. Suitable substituents include alkyl groups, —OH; —OC(O)R^{B} (wherein R^{B} is as defined herein), halogen groups and the like. The substituents on a substituted carbocyclic group are typically themselves unsubstituted, unless otherwise stated.

As used herein, a pharmaceutically acceptable salt is a salt with a pharmaceutically acceptable acid or base. Pharmaceutically acceptable acids include both inorganic acids such as hydrochloric, sulphuric, phosphoric, diphosphoric, hydrobromic or nitric acid and organic acids such as oxalic, citric, fumaric, maleic, malic, ascorbic, succinic, tartaric, benzoic, acetic, methanesulphonic, ethanesulphonic, benzenesulphonic or p-toluenesulphonic acid. Pharmaceutically acceptable bases include alkali metal (e.g. sodium or potassium) and alkali earth metal (e.g. calcium or magnesium) hydroxides and organic bases such as alkyl amines, aralkyl amines and heterocyclic amines. Hydrochloride salts and acetate salts are preferred, in particular hydrochloride salts.

In Formula (I), the stereochemistry is not limited unless otherwise specified. In particular, compounds of Formula (I) containing one or more chiral centre may be used in enantiomerically or diastereoisomerically pure form, or in the form of a mixture of isomers unless otherwise specified. Further, for the avoidance of doubt, such compounds may be used in any tautomeric form. Typically, the agent or substance described herein contains at least 50%, preferably at least 60, 75%, 90% or 95% of a compound according to Formula (I) which is enantiomerically or diasteriomerically pure. Typically, a compound of the invention comprises by weight at least 60%, such as at least 75%, 90%, or 95% of a single enantiomer or diastereomer. Preferably, the compound is substantially optically pure.

As used herein, a prodrug of a compound is a compound that readily undergoes chemical changes under physiological conditions to provide the active drug (the “parent compound”). Prodrugs can also be converted to the active drug compound by chemical or biochemical methods in an ex vivo environment. Prodrugs are typically pharmacologically inactive until converted into the active drug. Prodrugs are typically obtained by masking a functional group in the drug believed to be in part required for activity with a progroup to form a promoiety which undergoes a transformation, such as cleavage, under the specified conditions of use to release the functional group, and hence the active drug. Cleavage of the promoiety may proceed spontaneously (e.g. by hydrolysis) or may be induced by another (endogenous or exogenous) agent, e.g. exposure to an enzyme, light, acid or base, etc. Progroups are typically attached to the functional group of the active drug via bonds that are cleavable under specified conditions of use. A progroup is the portion of a promoiety that cleaves to release the functional group once administered to a subject. Progroups suitable for masking functional groups in active compounds are well-known in the art. A hydroxyl functional group may be masked as a sulfonate, ester (such as acetate or maleate) or carbonate promoiety, which may be hydrolyzed in vivo to provide the hydroxyl group. An amino functional group may be masked as an amide, carbamate, imine, urea, phosphenyl, phosphoryl or sulfenyl promoiety, which may be hydrolyzed in vivo to provide the amino group. A carboxyl group may be masked as an ester (including methyl, ethyl, pivaloyloxymethyl, silyl esters and thioesters), amide or hydrazide promoiety, which may be hydrolyzed in vivo to provide the carboxyl group.

As used herein, a derivative of a compound (e.g. an active drug) is a compound having a structure derived from the structure of a parent compound (e.g. a compound such as an SOCE facilitator disclosed herein) and whose structure is sufficiently similar to those disclosed herein and based upon that similarity, would be expected by one skilled in the art to exhibit the same or similar activities and utilities as the parent compound, or to induce, as a precursor, the same or similar activities and utilities as the parent compounds. Exemplary derivatives include salts, esters, amides, salts of esters or amides, pegylated derivatives of a parent compound and N-oxides of a parent compound.

SOCE Facilitators and Their Uses

As set out above, the invention provides a Store-Operated Ca²⁺ Entry (SOCE) facilitator for use in the treatment or prevention of viral infection in a subject. An SOCE facilitator is typically an SOCE activator. An SOCE facilitator can also be described as an SOCE inducer. A compound which causes SOCE, i.e. the activated function of the STIM-Orai complex (described above) in directing extracellular Ca²⁺ influx into the cell, is an SOCE facilitator as used herein. An SOCE facilitator (also known as an SOCE agonist) typically activates the ORAI channel to trigger extracellular Ca²⁺ influx into the cell. An SOCE facilitator may or may not cause ER calcium store depletion and ER stress. SOCE facilitators (and uses thereof) which activate the ORAI channel to trigger extracellular Ca²⁺ influx into the cell but which do not cause ER calcium store depletion and/or ER stress are within the scope of the invention. Typically, however, the SOCE facilitator does cause ER calcium store depletion and/or ER stress as well as activating the ORAI channel to trigger extracellular Ca²⁺ influx into the cell.

It will be apparent to those skilled in the art that the SOCE facilitator thus elevates intracellular calcium (Ca²⁺) levels. Accordingly, those skilled in the art will appreciate that SOCE may specifically refer to the activated function of the STIM-Orai complex in directing extracellular Ca²⁺ influx. CRAC entry relates to depletion of Ca²⁺ from the endoplasmic reticulum (ER) store and associated SOCE. Furthermore, a compound that facilitates transient SOCE during infection to induce a potent antiviral state may not necessarily cause overt extracellular Ca²⁺ influx during exposure to uninfected healthy cells.

Preferably, the SOCE facilitator is an inhibitor of the sarcoplasmic/endoplasmic reticulum Ca²⁺-ATPase (SERCA) pump. The SERCA pump resides in the endoplasmic/sarcoplasmic reticulum (ER) within myocytes. It is a Ca²⁺ ATPase that transfers Ca²⁺ from the cytosol of the cell to the lumen of the ER at the expense of ATP hydrolysis. The structure of purified SERCA derived from bovine muscle has been determined by X-ray crystallography (Sacchetto et al., 2012). Inhibitors of SERCA are known in the art, and can be identified by means such as by in vitro binding assays or by computational modelling of protein-ligand binding (molecular docking simulations). Known SERCA inhibitors include thapsigargin. Preferably, inhibition of the SERCA pump results in ER calcium store depletion and ensuing extracellular calcium influx. More preferably, inhibition of the SERCA pump results in ER calcium store depletion and extracellular calcium influx through activated SOCE. However, facilitation of SOCE without overt extracellular calcium influx at the point of administration can also be effective in virus inhibition. Calcium levels can be determined using methods known in the art such as fluorescence-based assays for detecting intracellular calcium mobilization.

Suitable assay kits are commercially available e.g. the Fluo-8 Ca²⁺ assay kit available from Abcam, used in accordance with its standard operating instructions.

The SOCE facilitator may also target other elements of the SOCE pathway without necessarily inhibiting SERCA. For example, the SOCE facilitator may activate one or more of Orai, STIM1, STIMATE and/or CRACR2A.

Preferably, in the invention, the SOCE facilitator inhibits progeny virus production from infected cells. Progeny virus production can be determined by methods known in the art such as immunodetection methods. For example, progeny virus production can be determined by immunodetection of viral nucleoprotein in MDCK cells infected with supernatant from virally-infected cells (e.g. using 6 h focus forming assays on MDCK cells), as described in Kuchipudi et al, Immunol. Cell Biol. 90:116-123 (2012). Preferably, progeny virus production (for example determined by 6 h focus forming assays) is reduced by at least 40%, e.g. at least 50%, for example at least 60%, e.g. at least 70%, more preferably at least 80% e.g. at least 90%, for example at least 95% or more, such as at least 96%, at least 97%, at least 98% or at least 99% compared to progeny virus production from untreated infected cells (i.e. cells which have not been treated with the SOCE facilitator, compound, composition or combination of the invention). Preferably, the SOCE facilitator, compound, composition or combination of the invention reduces progeny viral production from NPTr cells, primary porcine muscle cells [myoblasts], primary porcine tracheal epithelial cells [PTECs] and/or primary normal human bronchial epithelial [NHBE] cells, e.g. when measured by the methods disclosed in the examples. Preferably, the SOCE facilitator induces prolonged resistance of the host subject to viral infection. The prolonged resistance preferably last at least 4 hours, such as at least 6 hours, more preferably at least 8 hours e.g. at least 12 hours such as at least 24 hours, for example at least 48 hours e.g. at least 72 hours such as at least 96 hours or more.

Typically, the SOCE facilitator does not significantly decrease viral RNA expression. Viral RNA expression can be determined by extracting total RNA from infected cells using conventional means (e.g. RNeasy Plus Minikit, Qiagen) followed by cDNA synthesis (e.g. performed using Superscript III First Strand synthesis kit) with appropriate primers for viral RNA; e.g. primers for the viral M-gene.

Preferably, the SOCE facilitator inhibits virus replication in infected cells in the subject. Any infected cell type in the subject may be targeted. Preferably, the infected cells are infected respiratory epithelial cells or non-epithelial cells such as muscle cells; more preferably the infected cells are infected respiratory epithelial cells. In some embodiments, the infected cells are not kidney cells. The SOCE facilitator thus preferably inhibits virus replication in infected respiratory epithelial cells in the subject.

Preferably, the SOCE facilitator is a sesquiterpene or a pharmaceutically acceptable salt, derivative or prodrug thereof. More preferably, the SOCE facilitator is a sesquiterpene lactone or a pharmaceutically acceptable salt, derivative or prodrug thereof. A sesquiterpene is a compound typically derived from isoprene (CH₂═C(CH₃)—CH═CH₂) units. A sesquiterpene lactone is a compound typically derived from isoprene units, and incorporating a cyclic ester (lactone) group. A sesquiterpene or sesquiterpene lactone is typically cyclic and more typically comprises fused and/or bridged rings. Preferably, the sesquiterpene lactone is or is derived from a germacranolides, a heliangolide, a guaianolide, a pseudoguaianolide, a hypocretenolide or a eudesmanolide. Those skilled in the art will appreciate that a sesquiterpene or sesquiterpene lactone may comprise one or more double bond(s); one or more heteroatom(s) (such as O, N and/or S; preferably O) and may preferably be functionalised. For example, a sesquiterpene or sesquiterpene lactone may comprise one or more, such as from one to 10, e.g. 3 to 10 substituents (e.g. 3, 4, 5, 6, 7, 8, 9 or 10 substitutents) each preferably independently selected from unsubstituted C₁₋₂ alkyl; —OH; oxo; —C(O)—R^{B}; —R^{Z} and —C(O)R^{Z} wherein R^{B} and R^{Z} are each as described herein. As used herein, a sesquiterpene lactone may be functionalised at the lactone carbonyl moiety e.g. by replacing the carbonyl moiety with a C(H)—OR^{Y} group wherein R^{Y} is as described herein. Many sesquiterpenes and sesquiterpene lactones are known to the skilled person and are typically available commercially or can be derived from natural sources (e.g. plants) or synthesized or modified by known routes.

Preferably, in the invention, the SOCE facilitator is a compound of formula (I) or a pharmaceutically acceptable salt, derivative or prodrug thereof,

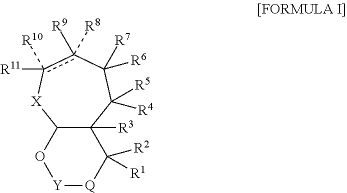

wherein:
X is selected from >C═R^{A}, >CH—R^{A} and —O—;
Y is selected from >C═O and >CH—OR^{Y};
R^{Y} is selected from H, R^{Z}, and —C(O)—R^{Z}; wherein R^{Z} is a C₁₋₂alkyl group and wherein R^{Z} is unsubstituted or is substituted with —COOH or —C₆H₄COOH;
Q is a bond or is CR¹²R¹³ wherein R¹² and R¹³ are each independently selected from H and methyl;
the moiety

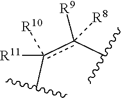

is selected from

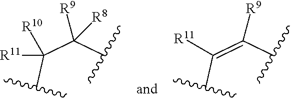

R⁵, R⁶ and R⁷ are each independently selected from H and methyl;
R⁹ is selected from H, —OH, unsubstituted C₁₋₂ alkyl and —OC(O)R^{B};
R⁸ and R¹⁰ if present are each independently selected from H and methyl;
Each R_{B} is independently selected from unsubstituted C₁₋₇ alkyl and unsubstituted C₂₋₇ alkenyl;

and wherein
when X is >C═R^{A} or >CH—R^{A}:
   R¹¹ is bonded to R^{A} to form, together with the atoms to which they are attached, a 5-membered carbocyclic group which is substituted by 2 to 4 groups independently selected from —OH, unsubstituted C₁₋₂ alkyl, oxo and —OC(O)R^{B};
   R¹ is selected from H and methyl and R² is selected from H, —OH and unsubstituted C₁₋₂ alkyl; or
   R¹ and R² together form a methylene moiety such that >CR¹R² is >C═CH₂;
   R³ is selected from H, —OH and unsubstituted C₁₋₂ alkyl;
   R⁴ is selected from H, —OH, unsubstituted C₁₋₂ alkyl and —OC(O)R^{B};

and
when X is O,
   R¹ is selected from H and methyl;
   R¹¹ is —O— and R³ is —O— and R¹¹ is bonded to R³ to form a —O—O— linker group;
   R⁴ is bonded to R² to form, together with the atoms to which they are attached, a 6-membered carbocyclic group which is substituted by 1 to 3 groups independently selected from —OH and unsubstituted C₁₋₂ alkyl.

Preferably, in Formula (I), R⁵ is H.

Preferably, in Formula (I), R⁶ is H.

Preferably, in Formula (I), R⁷ is H.

Preferably, in Formula (I), R⁹ is H, methyl or —OC(O)R^{B} wherein R^{B} is as defined herein. More preferably, R⁹ is H, methyl or —OC(O)R^{B} wherein R^{B} is unsubstituted C₁₋₇ alkyl, preferably methyl.

In Formula (I), Y is selected from >C═O and >CH—OR^{Y} wherein R^{Y} is selected from H, R^{Z}, and —C(O)—R^{Z}; wherein R^{Z} is a C₁₋₂ alkyl group and wherein R^{Z} is unsubstituted or is substituted with —COOH or —C₆H₄COOH. Preferably, when Y is >CH—OR^{Y}, R^{Y} is selected from H, unsubstituted C₁₋₂ alkyl and —C(O)—(C₂H₄)—COOH. More preferably, when Y is >CH—OR^{Y}, R^{Y} is selected from H and unsubstituted C₁₋₂ alkyl, preferably methyl. Still more preferably, when Y is >CH—OR^{Y}, R^{Y} is H.

Most preferably, Y is >C═O.

In Formula (I), Q is preferably a bond or is CHCH₃.

In Formula (I), the moiety

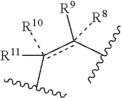

is selected from

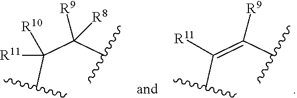

For avoidance of doubt, when the moiety

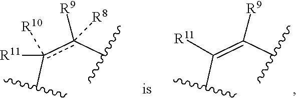

groups R⁸ and R¹⁰ are not present.

Preferably, therefore, the SOCE facilitator is a compound of formula (I) or a pharmaceutically acceptable salt, derivative or prodrug thereof, wherein
R⁵ is H;
R⁶ is H;
R⁷ is H;
R⁹ is H, methyl or —OC(O)R^{B} wherein R^{B} is as defined herein; more preferably R^{B} is unsubstituted C₁₋₇ alkyl, e.g. methyl;
Y is selected from >C═O and >CH—OR^{Y} wherein R^{Y} is selected from H, unsubstituted C₁₋₂alkyl and —C(O)—(C₂H₄)—COOH; more preferably R^{Y} is selected from H and unsubstituted C₁₋₂alkyl; most preferably Y is >C═O; and
Q is a bond or is CHCH₃.

In Formula (I), X is >C═R^{A} or >CH—R^{A}, or X is O. When X is >C═R^{A} or >CH—R^{A}, R¹¹ is preferably bonded to R^{A} to form, together with the atoms to which they are attached, a 5-membered carbocyclic group which is substituted by (i) two —OC(O)R^{B} groups and by one unsubstituted C₁₋₂ alkyl group; or (ii) one oxo group and one unsubstituted C₁₋₂ alkyl group. More preferably, when X is >C═R^{A} or >CH—R^{A}, R¹¹ is bonded to R^{A} to form, together with the atoms to which they are attached, (A) a 5-membered carbocyclic group which is substituted by (i) one methyl group; (ii) one —OC(O)—C₇H₁₅ group and (iii) one —OC(O)—C₄H₇ group; or (B) a 5-membered carbocyclic group which is substituted by (i) one oxo group and (ii) one methyl group.

Preferably, in one embodiment, when X is >C═R^{A} or >CH—R^{A}, R¹ is methyl and R² is selected from H, —OH and methyl; more preferably R¹ is methyl and R² is selected from H and —OH, most preferably —OH. Preferably, in another embodiment, when X is >C═R^{A} or >CH—R^{A}, R¹ and R² together form a methylene moiety such that >CR¹R² is >C═CH₂. Preferably, when X is >C═R^{A}, R¹ is methyl and R² is selected from H, —OH and methyl. Preferably, when X is >CH—R^{A}, R¹ and R² together form a methylene moiety such that >CR¹R² is >C═CH₂.

Preferably, when X is >C═R^{A} or >CH—R^{A}, R³ is selected from H, —OH and methyl, more preferably R³ is selected from H and —OH, most preferably R³ is —OH. Preferably, when X is >C═R^{A}, R³ is —OH. Preferably, when X is >CH—R^{A}, R³ is H.

Preferably, when X is >C═R^{A} or >CH—R^{A}, R⁴ is selected from H and —OC(O)R^{B}, preferably R⁴ is —OC(O)R^{B}. When R⁴ is —OC(O)R^{B}, R^{B} is preferably unsubstituted C₁₋₇ alkyl, preferably unsubstituted C₂₋₄ alkyl, more preferably C₃ alkyl. Preferably, when X is >C═R^{A}, R⁴ is —OC(O)R^{B}. Preferably, when X is >CH—R^{A}, R⁴ is H

Preferably, when X is >C═R^{A} or >CH—R^{A}, R⁸ when present is methyl. Preferably, when X is >C═R^{A} or >CH—R^{A}, R¹⁰ when present is H. Preferably, when X is >C═R^{A}, the moiety

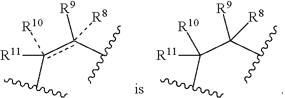

Preferably, when X is >CH—R^{A}, the moiety

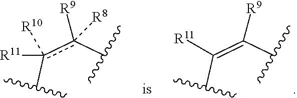

Preferably, when X is >C═R^{A} or >CH—R^{A}, R⁹ is unsubstituted C₁₋₂ alkyl, preferably methyl, or R⁹ is —OC(O)R^{B} wherein R^{B} is unsubstituted C₁₋₇ alkyl. Preferably, when X is >C═R^{A}, R⁹ is —OC(O)R^{B}. Preferably, when X is >CH—R^{A}, R⁹ is methyl.

Preferably, when X is >C═R^{A} or >CH—R^{A}, Q is a bond.

Preferably, therefore, the SOCE facilitator may be a compound of formula (I) or a pharmaceutically acceptable salt, derivative or prodrug thereof, wherein:
X is >C═R^{A} or >CH—R^{A};
R¹¹ is bonded to R^{A} to form, together with the atoms to which they are attached, a 5-membered carbocyclic group which is substituted by (i) two —OC(O)R^{B} groups and by one unsubstituted C₁₋₂ alkyl group or (ii) one oxo group and one unsubstituted C₁₋₂ alkyl group;
R¹ is methyl and R² is —OH; or
R¹ and R² together form a methylene moiety such that >CR¹R² is >C═CH₂;
R³ is selected from H and —OH;
R⁴ is selected from unsubstituted H and —OC(O)R^{B}, wherein R^{B} is preferably unsubstituted C₁₋₇ alkyl;
R⁵, R⁶ and R⁷ are each H;
R⁸ when present is methyl;
R⁹ is methyl or is —OC(O)R^{B} wherein R^{B} is unsubstituted C₁₋₇ alkyl;
R¹⁰ where present is H; and/or
Q is a bond.

Preferably, when X is >C═R^{A}, the SOCE facilitator is a compound of formula (II) or a pharmaceutically acceptable salt, derivative or prodrug thereof:

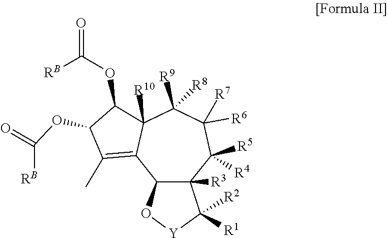

wherein Y, R¹, R², R³, R⁴, R⁵, R⁶, R⁷, R⁸, R⁹, R¹⁰ and R^{B} are each independently as described herein.

More preferably, when the SOCE facilitator is a compound of formula (II) or a pharmaceutically acceptable salt, derivative or prodrug thereof:
R¹ is methyl;
R² is selected from H and —OH;
R³ is selected from H and —OH;
R⁴ is —OC(O)R^{B}, wherein R^{B} is preferably unsubstituted C₂₋₄ alkyl;
R⁵ is H;
R⁶ is H;
R⁷ is H;
R⁸ is methyl;
R⁹ is —OC(O)R^{B} wherein R^{B} is C1₇ alkyl, e.g. methyl;
R₁₀ is H
Each R^{B} is independently unsubstituted C₁₋₇ alkyl (e.g. C₇ alkyl) or C₂₋₇ alkenyl (e.g. C₄ alkenyl); and
Y is selected from >C═O and >CH—OR^{Y} wherein R^{Y} is selected from H, unsubstituted C₁₋₂ alkyl and —C(O)—(C₂H₄)—COOH; more preferably R^{Y} is selected from H and unsubstituted C₁₋₂ alkyl; most preferably Y is >C═O.

Still more preferably, when X is >C═R^{A}, the SOCE facilitator is a compound of formula (IIa) or a pharmaceutically acceptable salt, derivative or prodrug thereof:

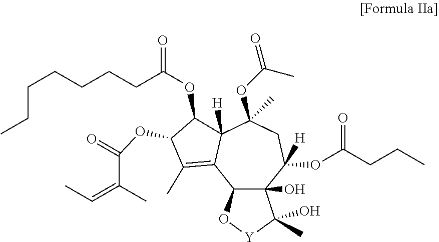

wherein Y is as described herein. Most preferably, when the SOCE facilitator is a compound of formula (IIa) or a pharmaceutically acceptable salt, derivative or prodrug thereof, Y is >C═O.

Preferably, when X is >CH—R^{A}, the SOCE facilitator is a compound of formula (III) or a pharmaceutically acceptable salt, derivative or prodrug thereof:

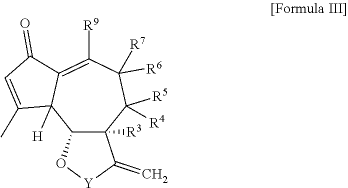

wherein Y, R³, R⁴, R⁵, R⁶, R⁷, and R⁹ are each independently as described herein.

More preferably, when the SOCE facilitator is a compound of formula (III) or a pharmaceutically acceptable salt, derivative or prodrug thereof:
R³ is H;
R⁴ is H;
R⁵ is H;
R⁶ is H;
R⁷ is H;
R⁹ is methyl; and
Y is selected from >C═O and >CH—OR^{Y} wherein R^{Y} is selected from H, unsubstituted C₁₋₂ alkyl and —C(O)—(C₂H₄)—COOH; more preferably R^{Y} is selected from H and unsubstituted C₁₋₂alkyl; most preferably Y is >C═O.

Still more preferably, when X is >CH—R^{A}, the SOCE facilitator is a compound of formula (IIIa) or a pharmaceutically acceptable salt, derivative or prodrug thereof:

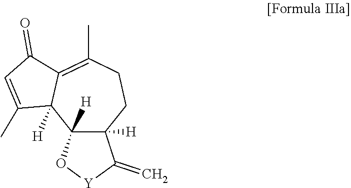

wherein Y is as described herein. Most preferably, when the SOCE facilitator is a compound of formula (IIIa) or a pharmaceutically acceptable salt, derivative or prodrug thereof, Y is >C═O.

When X is —O—, R¹¹ is —O— and R³ is —O— and R¹¹ is bonded to R³ to form a —O—O— linker group.

Preferably, when X is —O—, R¹ is H.

Preferably, when X is —O—, R⁴ is bonded to R² to form, together with the atoms to which they are attached, a 6-membered carbocyclic group which is substituted by 1 or 2 groups independently selected from —OH and unsubstituted C₁₋₂ alkyl. More preferably, when X is —O—, R⁴ is bonded to R² to form, together with the atoms to which they are attached, a 6-membered carbocyclic group which is substituted by 1 unsubstituted C₁₋₂ alkyl group, preferably methyl.

Preferably, when X is —O—, R⁸ is H.

Preferably, when X is —O—, R⁹ is H

Preferably, when X is —O—, R¹⁰ is methyl.

Preferably, when X is —O—, Q is CR¹²R¹³ wherein R¹² and R¹³ are each independently selected from H and methyl. More preferably, when X is —O—, Q is CHCH₃.

Preferably, therefore, the SOCE facilitator may be a compound of formula (I) or a pharmaceutically acceptable salt, derivative or prodrug thereof, wherein:
X is —O—;
R¹¹ is —O— and R³ is —O— and R¹¹ is bonded to R³ to form a —O—O— linker group;
R¹ is H;
R⁴ is bonded to R² to form, together with the atoms to which they are attached, a 6-membered carbocyclic group which is substituted by 1 unsubstituted C₁₋₂ alkyl group, preferably methyl;
R⁸ is H;
R⁹ is H;
R¹⁰ is methyl; and/or
Q is CR¹²R¹³ wherein R¹² and R¹³ are each independently selected from H and methyl.

Preferably, when X is —O—, the SOCE facilitator is a compound of formula (IV) or a pharmaceutically acceptable salt, derivative or prodrug thereof:

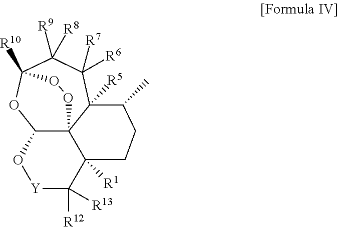

wherein Y, R¹, R⁵, R⁶, R⁷, R⁸, R⁹, R¹⁰, R¹² and R¹³ are each independently as described herein.

More preferably, when X is —O—, when the SOCE facilitator is a compound of formula (IV) or a pharmaceutically acceptable salt, derivative or prodrug thereof:
R¹ is H;
R⁵ is H;
R⁶ is H;
R⁷ is H;
R⁸ is H;
R⁹ is H;
R¹⁰ is methyl;
R¹² is H;
R¹³ is methyl; and
Y is selected from >C═O and >CH—OR^{Y} wherein R^{Y} is selected from H, unsubstituted C₁₋₂ alkyl and —C(O)—(C₂H₄)—COOH; more preferably R^{Y} is selected from H and unsubstituted C₁₋₂alkyl; most preferably Y is >C═O.

Still more preferably, when X is —O—, the SOCE facilitator is a compound of formula (IVa) or a pharmaceutically acceptable salt, derivative or prodrug thereof:

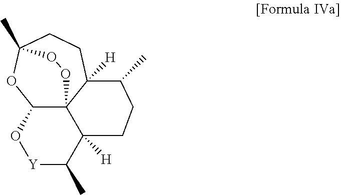

wherein Y is as described herein. Most preferably, when the SOCE facilitator is a compound of formula (IVa) or a pharmaceutically acceptable salt, derivative or prodrug thereof, Y is >C═O.

Preferably, the SOCE facilitator is thapsigargin, artemisinin, (+)-ledene, dehydroleucodine, or valerenic acid; or a pharmaceutically acceptable salt, derivative or prodrug of is thapsigargin, artemisinin, (+)-ledene, dehydroleucodine, or valerenic acid. Most preferably, the SOCE facilitator is thapsigargin or artemisinin or a pharmaceutically acceptable salt, derivative or prodrug of thapsigargin or artemisinin. The structures of thapsigargin, artemisinin, (+)-ledene, dehydroleucodine, and valerenic acid are shown below. Most preferably, the SOCE facilitator is thapsigargin.

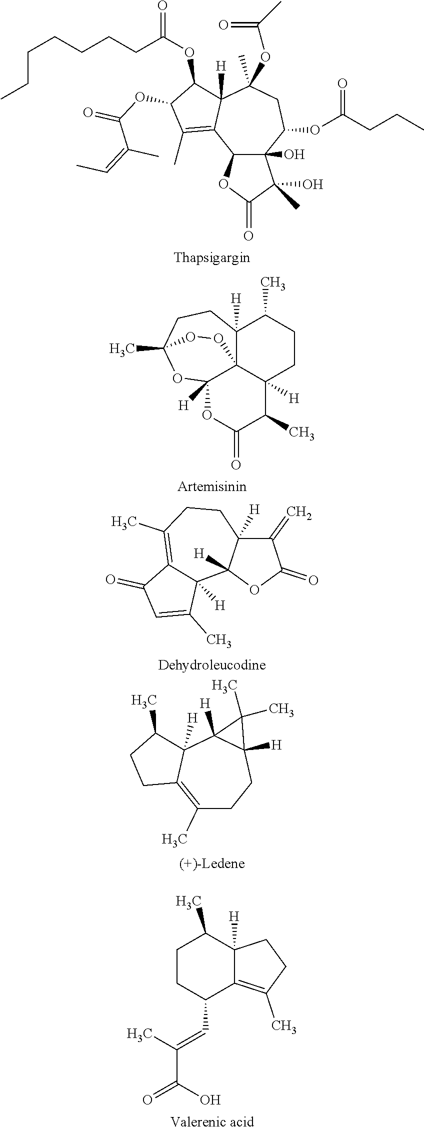

The invention also provides a compound for use in the treatment or prevention of viral infection in a subject in need thereof, wherein said compound is a sesquiterpene or sesquiterpene lactone. Preferably, the sesquiterpene or sesquiterpene lactone is a compound of Formula (I) as defined above. More preferably, the sesquiterpene or sesquiterpene lactone is a compound of Formula (II), (III) or (IV) as defined above. Most preferably, the sesquiterpene or sesquiterpene lactone is a compound of Formula (IIa), (IIIa) or (IVa) as defined above. Preferred sesquiterpenes and sesquiterpene lactones are thapsigargin, artemisinin, (+)-ledene, dehydroleucodine, and valerenic acid; and pharmaceutically acceptable salts, derivatives and prodrugs of thapsigargin, artemisinin, (+)-ledene, dehydroleucodine, and valerenic acid. More preferred sesquiterpenes and sesquiterpene lactones are thapsigargin and artemisinin and pharmaceutically acceptable salts, derivatives and prodrugs thereof. Thapsigargin is most preferred.

Synthesis

An SOCE facilitator in accordance with the invention can be prepared by any suitable method. SOCE facilitators as defined herein are known in the art or are commercially available, or can be synthesized by known methods. For example, compounds of Formula (I) can typically be isolated from natural products such as from plants e.g. Artemisia annua, Artemisia douglasiana or Thapsia garganica. Chemical synthesis of compounds of Formula (I) is also possible. Common starting points include artemisinic acid. Ring closure reactions known to the skilled chemist can be employed to produce the compounds of Formula (I). Functionalisation is achieved via known routes; for example ester groups can readily by obtained by reaction of a hydroxy group with an appropriate carboxylic acid or activated form thereof. Suitable alcohol protecting groups are well known to those skilled in the art and include benzyl (Bn); [bis-(4-methoxyphenyl)phenylmethyl] (DMT); Tetrahydrofuran (THF); trimethylsilyl (TMS), tert-butyldimethylsilyl (TBDMS), tri-iso-propylsilyloxymethyl (TOM), and triisopropylsilyl (TIPS) ether protecting groups, and the like. Exemplary compounds of Formula (I) are compounds of Formula (II), (III) and (IV) above. Compounds of Formula (II) can be readily synthesized from commercially available materials such as thapsigargin. Compounds of Formula (III) can be readily synthesized from commercially available materials such as dehydroleucodine. Compounds of Formula (IV) can be synthesized from commercially available materials such as artemisinic acid. The compounds of Formula (I) are sesquiterpene lactones. The lactone group can be selectively reduced with hydride-reducing agents, such as sodium borohydride, potassium borohydride, and lithium borohydride, to yield the dihydro lactol form (i.e. when Y is >CH—OR^{Y} and R^{Y} is H). This form can be further functionalised (e.g. when Y is >CH—OR^{Y} and R^{Y} is selected from R^{Z}, and —C(O)—R^{Z} wherein R^{Z} is as defined herein) by reaction of the dihydro lactol form with appropriate reagents such as with R^{Z}—COOH. Artemisinin, dehydroleucodine and thapsigargin are both commercially available e.g. from Sigma Aldrich and can be used as starting materials for the production of compounds of Formula (I), (II), (IIa), (III), (IIIa), (IV) and (IVa) as set out above. Ledene (also known as (+)-ledene) and valerenic acid are commercially available e.g. from Sigma Aldrich.

Therapeutic Efficacy

As will be apparent from the above discussion, the SOCE facilitators, sesquiterpenes and sesquiterpene lactones used in accordance with the present invention are therapeutically useful. The invention therefore also provides the use of an SOCE facilitator or a compound which is a sesquiterpene or sesquiterpene lactone as described herein in the manufacture of a medicament for treating or preventing viral infection in a subject in need thereof. The invention also provides methods of treating or preventing viral infection using an SOCE facilitator or a compound which is a sesquiterpene or sesquiterpene lactone as described herein. For the avoidance of doubt, the SOCE facilitators used in accordance with the present invention may be administered in the form of a solvate.

Also provided is a pharmaceutical composition for use in the treatment of prevention of viral infection in a subject in need thereof comprising a compound as defined herein together with a pharmaceutically acceptable carrier or diluent and optionally further comprising another antiviral agent. Typically, the composition contains up to 50 wt % of the compound. More typically, it contains up to 20 wt % of the compound, e.g. up to 10 wt % for example up to 1 wt % e.g. up to 0.1 wt % such as up to 0.01wt % e.g. up to 0.001 wt % or less. Compositions comprising low amounts (wt %) of the compound are particularly appropriate for highly active compounds. Preferred pharmaceutical compositions are sterile and pyrogen free. Further, when the pharmaceutical compositions provided by the invention contain a compound which is optically active, the compound is typically a substantially pure optical isomer.

The composition of the invention may be provided as a kit comprising instructions to enable the kit to be used in the methods described herein or details regarding which subjects the method may be used for.

As explained above, the compound used in the present invention is useful in treating or preventing viral infection in a subject in need thereof. In particular, they are inhibitors of RNA viruses.

An SOCE facilitator or compound which is a sesquiterpene or sesquiterpene lactone as described herein or may be used as a standalone therapeutic agent. For example, an SOCE facilitator or sesquiterpene or sesquiterpene lactone as described herein may be used as a standalone adjunct in antiviral therapy. Alternatively, it may be used in combination with other antiviral agents to enhance the action of the other antiviral agent. The SOCE facilitator, sesquiterpene or sesquiterpene lactone may find particular use in treating or preventing viral infection caused by viruses which are resistant to treatment with conventional antiviral agents (e.g. amantadine, remantadine, zanamivir, oseltamivir, laninamivir and peramivir) when administered alone. Treatment or prevention of such infection with conventional antiviral agents alone may be unsuccessful.

The present invention therefore also provides a combination comprising (i) an SOCE facilitator as defined herein or a compound which is a sesquiterpene or sesquiterpene lactone as described herein and (ii) an additional antiviral agent. The SOCE facilitator or compound which is a sesquiterpene or sesquiterpene lactone and the additional antiviral agent may be provided in a single formulation, or they may be separately formulated.

Where separately formulated, the two agents may be administered simultaneously or separately. They may be provided in the form of a kit, optionally together with instructions for their administration. The products may also be referred to herein as products or pharmaceutical combinations.

Where formulated together, the two active agents may be provided as a pharmaceutical composition comprising (i) an SOCE facilitator as described herein or a compound which is a sesquiterpene or sesquiterpene lactone as defined herein and (ii) an additional antiviral agent; and (iii) a pharmaceutically acceptable carrier or diluent.

Preferably, the additional antiviral agent is an anti-neuraminidase antiviral agent or an antiviral agent that inhibits the viral M2 protein. More preferably, the antiviral agent is an anti-neuraminidase antiviral agent. Preferably, the antiviral agent is selected from amantadine, rimantadine, zanamivir, oseltamivir, laninamivir and peramivir, or a pharmaceutically acceptable salt of any of the preceding agents.

The combinations of the invention are also useful in treating or preventing viral infection. The present invention therefore provides a combination of the invention for use in medicine. The present invention also provides a combination of the invention for use in treating or preventing viral infection in a subject in need thereof. The invention also provides the use of a combination of the invention in the manufacture of a medicament; e.g. a medicament for treating or preventing viral infection in a subject in need thereof. The invention also provides methods of treating or preventing viral infection using the combinations of the invention.

In one aspect, the subject is a mammal, in particular a human. However, it may be non-human. Preferred non-human animals include, but are not limited to, primates, such as marmosets or monkeys, commercially farmed animals, such as horses, cows, sheep or pigs, and pets, such as dogs, cats, mice, rats, guinea pigs, ferrets, gerbils or hamsters. The subject may also be a bird. Preferred birds include commercially farmed birds such as chickens, geese, ducks, turkeys, pigeons, ostriches and quail. The subject can be any animal that is capable of being infected by a virus.

The compounds, compositions and combinations described herein are useful in the treatment of viral infection which occurs after a relapse following an antiviral treatment. The compounds, compositions and combinations can therefore be used in the treatment of a patient who has previously received antiviral treatment for the (same episode of) viral infection.

The virus causing the infection may be any infective virus. Typically the virus is an RNA virus. Typically the virus is not a DNA virus. More typically, the virus is an influenza virus, such as an influenza A virus. The virus may be a virus of the Paramyxoviridae family e.g. respiratory syncytial virus (RSV virus). Preferably, the virus does not involve in its replication cycle the maturation of progeny viral particles in the endoplasmic reticulum (ER). Influenza viruses and viruses in the Paramyxoviridae family (e.g. RSV virus) do not involve viral accumulation in the ER. Preferably, the virus is not a rotavirus such as a porcine rotavirus. Typically, the viral infection to be treated as described herein is resistant to treatment with a conventional antiviral agent when the conventional antiviral agent is used alone.

The viral infection may, for example, be caused by a human influenza virus such as a human influenza A virus, an avian influenza virus such as an avian influenza A virus, or a porcine influenza virus such as a porcine influenza A virus. The virus may be an epidemic or pandemic strain. The infection may be caused by a virus of strain H1N1, (e.g. pandemic 2009 H1N1), H3N2, H5N1, H5N6 or H7N9 viruses.

The virus may be a virus of the Paramyxoviridae family. Typically, a virus of the Paramyxoviridae family is selected from RSV, parainfluenza virus, measles virus and henipaviruses. Preferably the virus of the Paramyxoviridae family is RSV, such as human RSV. Experiments into the effect of compounds such as TG on RSV viruses have previously been conducted only at toxic levels of the compound leading to decreased cell viability (Cui et al, 2016), such that any observed decrease in infectivity can be assigned to apoptosis and/or cytotoxicity, rather than inhibition of viral replication and/or infectivity. The inventors have surprisingly found that a virus such as RSV can be targeted with SOCE facilitators such as TG at non-toxic levels leading to a viable treatment for such infections.

The compound, composition or combination described herein may be used to treat or prevent infections and conditions caused by any one or a combination of the above-mentioned viruses. In particular, the compound, composition or combination described herein may be used in the treatment or prevention of influenza.

A compound, composition or combination as described herein can be administered to the subject in order to prevent the onset or reoccurrence of one or more symptoms of the viral infection. This is prophylaxis. In this embodiment, the subject can be asymptomatic. The subject is typically one that has been exposed to the virus. A prophylactically effective amount of the agent or formulation is administered to such a subject. A prophylactically effective amount is an amount which prevents the onset of one or more symptoms of the viral infection.

A compound, composition or combination described herein can be administered to the subject in order to treat one or more symptoms of the viral infection. In this embodiment, the subject is typically symptomatic. A symptomatic subject may exhibit one or more of the symptoms of viral infection e.g. infection by influenza virus. For example, the subject may have one or more symptoms selected from fever and/or chills; cough; nasal congestion; rhinorrhea; sneezing; sore throat; hoarseness (dysphonia); respiratory distress; ear pressure; earache; muscle ache; fatigue; headache; irritated eyes; reddened eyes, skin (especially face), mouth, throat and/or nose; petechial rash and/or gastrointestinal symptoms such as diarrhoea, vomiting, and/or abdominal pain. A therapeutically effective amount of the agent or formulation is administered to such a subject. A therapeutically effective amount is an amount effective to ameliorate one or more symptoms of the disorder.

The present invention is particularly advantageous in the medical setting. A compound, composition or combination described herein can be administered to a subject following diagnosis of a viral infection, such as infection by an influenza virus. Alternatively, a compound, composition or combination described herein may be administered to a subject wherein viral infection has not previously been diagnosed. The determination of whether or not viral infection such as by an influenza virus is present may be made in the context of any disease or illness present or suspected of being present in a patient. Such diseases may include those caused by, linked to, or exacerbated by the presence of the virus. Thus, a patient may display symptoms indicating the presence of viral infection (e.g. by the influenza virus), such as a respiratory illness, and a sample may be obtained from the patient in order to determine the presence of the virus and optionally also the serotype, subtype or strain thereof. The serotype, subtype or strain of the virus can be determined by serology, immunoassay or viral culture from a sample provided by the subject. Diagnosis can also be performed based on nucleic acid derived from a sample of a patient, providing an indication to clinicians whether an illness for example a respiratory illness is due to a viral infection e.g. by influenza virus.

The compound, composition or combination may be administered in a variety of dosage forms. Thus, it can be administered orally, for example as tablets, troches, lozenges, aqueous or oily suspensions, dispersible powders or granules. Formulations of the compound, composition or combination may also be administered parenterally, whether subcutaneously, intravenously, intramuscularly, intrasternally, transdermally or by infusion techniques. Preferably, the compound, composition or combination may be administered via inhaled (aerosolised) or oral administration, most preferably by inhaled (aerosolised) administration.

The compound, composition or combination is typically formulated for administration with a pharmaceutically acceptable carrier or diluent. For example, solid oral forms may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, arabic gums, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non-toxic and pharmacologically inactive substances used in pharmaceutical formulations. Such pharmaceutical preparations may be manufactured in known manner, for example, by means of mixing, granulating, tableting, sugar coating, or film coating processes.

The compound, composition or combination may be formulated for pulmonary administration. For example, the compound, composition or combination may be formulated for inhaled (aerosolised) administration as a solution or suspension. The compound, composition or combination may be administered by a metered dose inhaler (MDI) or a nebulizer such as an electronic or jet nebulizer. Alternatively, the compound, composition or combination may be formulated for inhaled administration as a powdered drug; such formulations may be administered from a dry powder inhaler (DPI). When formulated for inhaled administration, the compound, composition or combination may be delivered in the form of particles which have a mass median aerodynamic diameter (MMAD) of from 1 to 100 μm, preferably from 1 to 50 μm, more preferably from 1 to 20 μm such as from 3 to 10 μm, e.g. from 4 to 6 μm. When the compound, composition or combination is delivered as a nebulized aerosol, the reference to particle diameters defines the MMAD of the droplets of the aerosol. The MMAD can be measured by any suitable technique such as laser diffraction.

Accordingly, the invention also provides an aerosol formulation comprising an SOCE facilitator as defined herein or a compound which is a sesquiterpene or sesquiterpene lactone. The SOCE facilitator, sesquiterpene or sesquiterpene lactone may preferably be a compound of Formula (I) as defined herein, or a pharmaceutically acceptable salt, derivative or prodrug thereof. More preferably, the SOCE facilitator, sesquiterpene or sesquiterpene lactone in the aerosol formulation may be selected from a compound of Formula (II) (e.g. Formula (IIa)) as defined herein; or a compound of Formula (III) (e.g. Formula (IIIa)) as defined herein; or a compound of Formula (IV) (e.g. Formula (IVa)) as defined herein; or a pharmaceutically acceptable salt, derivative or prodrug thereof. Most preferably, the SOCE facilitator, sesquiterpene or sesquiterpene lactone in the aerosol formulation may be selected from thapsigargin, artemisinin, (+)-ledene, dehydroleucodine, and valerenic acid or a pharmaceutically acceptable salt, derivative or prodrug thereof, most preferably artemisinin or thapsigargin or a pharmaceutically acceptable salt, derivative or prodrug of artemisinin or thapsigargin.

Liquid dispersions for oral administration may be syrups, emulsions and suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspension or solutions for intramuscular injections or inhalation may contain, together with the active compound, a pharmaceutically acceptable carrier, e.g. sterile water, olive oil, ethyl oleate, glycols, e.g. propylene glycol, and if desired, a suitable amount of lidocaine hydrochloride.

Solutions for inhalation, injection or infusion may contain as carrier, for example, sterile water or preferably they may be in the form of sterile, aqueous, isotonic saline solutions. Pharmaceutical compositions suitable for delivery by needleless injection, for example, transdermally, may also be used.

A therapeutically or prophylactically effective amount of the SOCE facilitator, sesquiterpene or sesquiterpene lactone is administered to a subject. The dose may be determined according to various parameters, especially according to the compound used; the age, weight and condition of the subject to be treated; the route of administration; and the required regimen. Again, a physician will be able to determine the required route of administration and dosage for any particular subject. A typical daily dose is from about 1 ng to 100 mg per kg; for example from about 0.01 to 100 mg per kg, preferably from about 0.1 mg/kg to 50 mg/kg, e.g. from about 1 to 10 mg/kg of body weight, according to the activity of the specific inhibitor, the age, weight and conditions of the subject to be treated, the type and severity of the disease and the frequency and route of administration. Preferably, daily dosage levels are from 5 mg to 2 g.

When the SOCE facilitator is TG or a pharmaceutically acceptable salt thereof, a typical daily dose may be from about 1 ng/kg to about 10 μg/kg of body weight; e.g. from about 8 ng/kg to 2 μg/kg, e.g. from about 0.1 μg/kg to about 1 μg/kg according to the age, weight and conditions of the subject to be treated, the type and severity of the disease and the frequency and route of administration, and may preferably be administered by inhalation.

When the SOCE facilitator, sesquiterpene or sesquiterpene lactone is administered to a subject in combination with another active agent (for example in the form of a pharmaceutical combination comprising an additional antiviral agent), the dose of the other active agent (e.g. additional antiviral agent) can be determined as described above. The dose may be determined according to various parameters, especially according to the agent used; the age, weight and condition of the subject to be treated; the route of administration; and the required regimen. Again, a physician will be able to determine the required route of administration and dosage for any particular subject. A typical daily dose is from about 0.01 to 100 mg per kg, preferably from about 0.1 mg/kg to 50 mg/kg, e.g. from about 1 to 10 mg/kg of body weight, according to the activity of the specific inhibitor, the age, weight and conditions of the subject to be treated, the type and severity of the disease and the frequency and route of administration. Preferably, daily dosage levels are from 5 mg to 2 g.

The dose is preferably administered transiently. The frequency of the administration of the dose may be determined according to various parameters, especially according to the compound used; the age, weight and condition of the subject to be treated; the route of administration; and the required regimen. Again, a physician will be able to determine the required frequency of administration for any particular subject and dosage. A typical frequency of administration is from about once per month to about 5 times per day, e.g. from about once per week to about 3 times per day, such as from about twice per week to about twice per day, e.g. once every other day or once daily, according to the activity of the specific inhibitor, the age, weight and conditions of the subject to be treated, the type and severity of the disease and the route of administration. The frequency and duration of the administration of the dose may be determined to provide an effective priming of the target cells in the subject to prevent or treat viral infection.

When multiple doses are required, the frequency of dosing may be controlled such that successive doses are administered to the patient when the plasma concentration of the

SOCE facilitator, sesquiterpene or sesquiterpene lactone has decreased to at most 50% of the peak concentration following the previous dose, such as at most 25% of the peak concentration, e.g. at most 10% of the peak concentration, such as at most 5% of the peak concentration, e.g. at most 2% of the peak concentration, for example at most 1% of the peak concentration. Such dosage regimens reflect the finding of the present inventors that the SOCE facilitators described herein typically exhibit a sustained antiviral response following their administration.

Those skilled in the art will appreciate that the dose of the SOCE facilitator, sesquiterpene or sesquiterpene lactone administered to the subject is non-toxic to the subject. The dose is preferably determined in order to provide the desired antiviral effect without inducing cytotoxic effects. The dose is thus preferably determined in order to provide the desired antiviral effect without causing cell death or without increasing cytotoxicity. A physician will be able to determine an appropriate dose according to various parameters, especially according to the compound used; the age, weight and condition of the subject to be treated; and the route of administration.

In Vitro Methods

The invention also provides an in vitro method of evaluating the antiviral activity or potential antiviral activity of a compound against a virus. The in vitro method comprises assessing the activity of the compound to activate CRAC entry mediated SOCE. Preferably, the in vitro method further comprising assessing the activity of the compound to prevent infection of cells by the virus.

The in vitro method may involve using a fluorescence-based assay for detecting intracellular calcium mobilization to assess the activity of the compound to activate CRAC entry mediated SOCE. Suitable fluorescence-based assays for detecting intracellular calcium mobilization are known in the art and include the Fluo-8 Ca²⁺ assay kit available from Abcam, used in accordance with its standard operating instructions. The in vitro method may involve using a hemagglutination assay to assess the activity of the compound to reduce virus production from infected cells. Protocols for conducting hemagglutination assays are well known in the art.

The in vitro method may involve evaluating the antiviral activity or potential antiviral activity of the compound by comparing the extent to which the compound (i) activates CRAC entry mediated SOCE and optionally (ii) reduces infection of cells by the virus with that of a reference compound. Preferably, the reference compound is an SOCE facilitator as defined herein. The in vitro method may involve the high-throughput screening of multiple compounds. Typically, in the in vitro method of the invention, the virus is an RNA virus, preferably an influenza virus, more preferably an influenza A virus.

The following Examples illustrate the invention. They do not however, limit the invention in any way. In this regard, it is important to understand that the particular assay used in the Examples section is designed only to provide an indication of biological activity. There are many assays available to determine biological activity, and a negative result in any one particular assay is therefore not determinative.

### EXAMPLES

### Example 1: Materials and Method

Cells and Influenza A Viruses

Primary NHBE cells from three different donors and bronchial epithelial growth media were supplied by Promocell. PTECs were isolated from stripped tracheobronchial mucosae from eight 3- to 4-month-old pigs. Briefly, washed mucosae were incubated at 4° C. overnight with 0.06 U/ml pronase (Sigma) in a 1:1 dilution of Dulbecco's modified Eagle's medium (DMEM)-F12 medium. Supernatants containing cells were centrifuged and washed in DMEM-Glutamax (high glucose) (Invitrogen) and subsequently cultured in bronchial epithelial growth media (Promocell). Skeletal muscle cells were isolated and cultured as previously described (Sebastian et al., 2015). Immortalised NPTr cells were cultured in DMEM-Glutamax supplemented with 10% foetal calf serum (FCS) and 100 U/ml penicillin-streptomycin (P/S). MDCK cells were grown in DMEM-Glutamax (high glucose) supplemented with 10% FCS and 100 U/ml P/S. A human pandemic (pdm) H1N1 2009 (A/California/07/2009) and a human USSR H1N1 (A/USSR/77) were used in this study. All viruses were propagated in 10-day-old embryonated chicken eggs and allantoic fluid was harvested at 48 h post inoculation. Virus was aliquoted and stored in −80° C. until further use.

Cell Viability and Caspase 3/7 Assays

Cells were primed for 30 min with the relevant compound (e.g. TG or other compounds) as indicated in specific experiments, rinsed three times with phosphate buffered saline (PBS) and cultured overnight. Cell viability based on the detection of ATP was determined using a CellTiter-Glo Luminescent Cell Viability Assay kit (Promega), and activated caspase 3 and 7 were quantified using a Caspase-Glo 3/7 Assay (Promega) kit according to manufacturer's instructions.

TG Priming of Cells

The concentrations of TG used to prime each cell type were chosen for no detectable cytotoxic effect on cell viability, based on host ATP production and caspase 3/7 activity. For TG priming pre-infection, cells were cultured in the presence of TG, typically for 30 mins, rinsed three times with PBS and followed by influenza virus infection as described below. For TG priming during infection, cells were first infected for 6 h, rinsed with PBS, primed with TG for 30 min, rinsed again three times with PBS and cultured overnight (24 h culture) in infection media. Spun supernatants were used for virus titration in MDCK cells as described below.

Infection and Progeny Virus Quantification

Infection medium for NPTr cells and pig muscle cells (myoblasts and myotubes) was Ultraculture medium (Lonza) supplemented with 100 U/ml P/S, 2 mM glutamine and 250 ng/ml L-1-tosylamide-2-phenylethyl chloromethyl ketone (TPCK) trypsin (Sigma). Infection medium of primary cells (PTECs and NHBE cells) was bronchial epithelial growth medium (Promocell) supplemented with 250 ng/ml TPCK trypsin. Cells were infected at specified multiplicity of infection (MOI) of influenza virus for 2 h in infection media, after which they were rinsed three times with PBS and incubated in fresh infection media for a further 22 h. MOI of 1.0 is the minimum volume of virus needed to infect all MDCK cells in a culture well. Quantification of infectious virus in spun culture supernatants was conducted as previously described (Kuchipudi et al., 2012) which was an immuno-cytochemical focus forming assay based on infection of MDCK cells with harvested supernatants for 6 h followed by immunodetection of viral nucleoprotein (NP). Briefly, MDCK cells infected for 6 h were fixed in acetone methanol for 10 min followed by peroxidase treatment for 10 min and incubation with a 1:8000 dilution of primary mouse monoclonal antibody to influenza nucleoprotein (Abcam) for 40 min at room temperature. The cells were subsequently rinsed with Tris-buffered saline (TBS), incubated with horse radish peroxidase-labelled polymer for 40 min. After gently rinsing with TBS, the cells were incubated with DAB substrate-chromogen solution for 7 min (Envision+system-HRP kit, Dako). Cells positive for viral nucleoprotein were counted with an inverted microscope and the mean of positive cells in four 96-wells was used to calculate infectious focus-forming units of virus per microlitre of infection volume.

Intracellular Ca²⁺ Monitoring Assay

Intracellular Ca²⁺ was measured using the Fluo-8 No Wash Calcium Assay Kit (Abcam) according to manufacturer's instructions. Briefly, cells were grown until confluent in Nunc Microwell 96-well optical-bottom plates (black) with polymer base. The culture media were replaced with 100 μl of DMEM-Glutamax containing 1% FCS. Fluo-8 dye loading solution was further added at 100 μl to each well and incubated at room temperature for 1 h whereupon TG was added at specified concentrations for 10 min. Fluorescence intensity was measured at Ex/Em=490/525 nm.

RNA Preparation and Real-Time RT-PCR

Total RNA was extracted from cells using an RNeasy Plus Minikit (Qiagen). cDNA was synthesized from 0.5 or 1 μg of total RNA using Superscript III First Strand synthesis kit (Invitrogen). Expression of host genes was performed with a LightCycler-96 instrument (Roche), and computation was based on the comparative Ct approach, normalised to 18 S ribosomal RNA. Primer sequences for human RIG-I (DDX58) were 5′-GAAGG CATTG ACATT GCACA GT-3′ fwd primer and 5′-TGGTT TGGAT CATTT TGATG ACA-3′ rev primer. Human ER stress primers for DDIT3 (FH1_DDIT3 and RH1_DDIT3), HSPA5 (FH1_HSPA5 and RH1_HSPA5) and HSP90B1 (FH1_HSP90B1 and RH1_HSP90B1), human IFNB1 primers (FH1_IFNB1 and RH1_IFNB1), and human OAS1 primers (FH1_OAS1 and RH1_OAS1) were pre-made designs from Sigma-Aldrich. Primer sequences for pig RIG-I were 5′-CCCTG GTTTA GGGAC GATGA G-3′ fwd primer and 5′-AACAG GAACT GGAGA AAAGT GA-3′ rev primer, for pig OAS1 were 5′-GAGCT GCAGC GAGAC TTCCT-3′ (Pig OAS1-Forward 2) and 5′-GGCGG ATGAG GCTCT TCA-3′ (Pig OAS1-Reverse 2), and for pig PKR were 5′-TCTCC CACAA CGAGC ACATC-3′ fwd primer and 5′-ACGTA TTTGC TGAGA AGCCA TTT-3′ rev primer. Pig ER stress primers for DDIT3 (FSUS1_DDIT3 and RSUS1_DDIT3), HSPA5 (FSUS1_HSPA5 and RSUS1_HSPA5) and HSP90B1 (FSUS1_HSP90B1 and RSUS1_HSP90B1), and pig IFNB1 primers (FSUS_IFNB1 and RSUS1_IFNB1) were pre-made designs from Sigma-Aldrich. Primer sequences for USSR H1N1 virus M-gene were 5′-AGATG AGCCT TCTAA CCGAG GTCG-3′ fwd primer and 5′-TGCAA AAACA TCTTC AAGTC TCTG-3′ rev primer, and for pdm H1N1 virus M-gene were 5′-AGATG AGTCT TCTAA CCGAG GTCG fwd primer and 5′-TGCAA AGACA CTTTC CAGTC TCTG-3′ rev primer.

Western Blotting

Cells were lysed by radioimmunoprecipitation assay (RIPA) buffer (Santa Cruz) supplemented with 1% phenylmethylsulfonyl fluoride (PMSF) (Santa Cruz), 1% inhibitor cocktail and 1% sodium orthovanadate (Santa Cruz). Protein concentration was determined by Bio-RAD protein assay (Bio-Rad). Primary antibodies were goat anti-viral M1 at 1:500 dilution (Abcam, ab20910), rabbit anti-viral NP at 1:500 dilution (Thermo Scientific, PAS-32242) and mouse anti-β-actin at 1:10000 (Sigma, A5316), and appropriate species-specific secondary antibodies were peroxidase-conjugated.

STIM1 and Orai1 Gene Knockdown

NHBE cells were transfected with 10 pmol/ml siRNA, using the minimum recommended volume of transfection reagent Lipofectamine RNAiMAX (Invitrogen) according to manufacturer's protocol. Pre-designed Silencer® Select siRNAs against ORAI1 (siRNA ID s228396), STIM1 (siRNA ID s531229) and the Silencer™ Select Negative Control No. 1 siRNA (Invitrogen) were used. Cells were exposed to the siRNA-lipid complex for 6 h before washing with PBS and cultured in fresh media. They were subsequently infected with influenza virus 48 h post transfection.

Electron Microscopy

Samples for transmission electron microscopy were fixed in 3% glutaraldehyde in 0.1 M sodium cacodylate buffer (pH 7.4) for 1 h. The samples were then post fixed in 1% osmium tetroxide, dehydrated graded ethanol series of 50, 70, 90 and 100%, dried further in a transitional solvent, propylene oxide, infiltrated in epoxy resin and polymerised in an embedding oven at 60° C. for 48 h. Ultrathin sections were collected using a diamond knife on a Leica UC6 ultramicrotome, at 80 nm from each block, mounted on 200 mesh copper grids and stained in uranyl acetate and lead citrate. Grids were visualised on a Tecnai T12 Biotwin TEM, which ran at an accelerating voltage of 110 kV, and images were captured using a Mega View SIS camera.

Statistical Analysis

Statistical analysis was performed using GraphPad Prism 6 (GraphPad Software). Paired student t test, one-way ANOVA and two-way ANOVA were used to test differences between different groups. P values <0.05 were considered significant.

### Example 2

This example shows that raising extracellular Ca²⁺ in the culture of different cell types reduced influenza virus output.

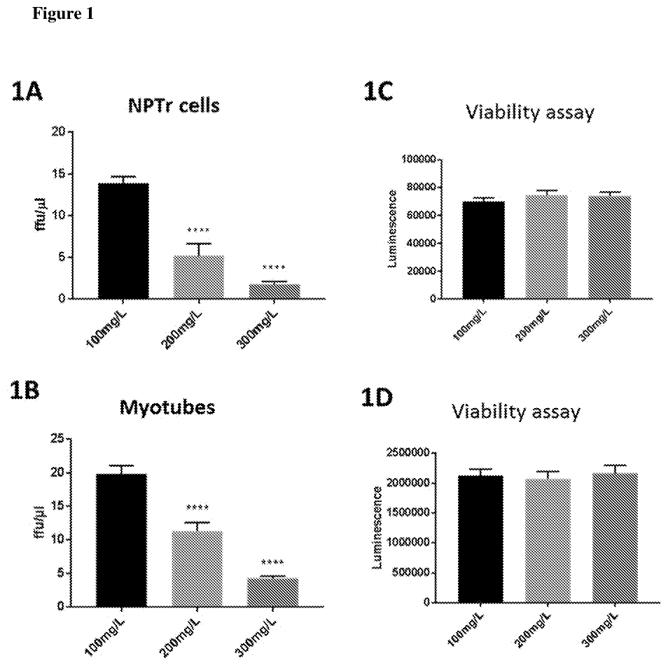

Experiments were conducted to assess the effect of extracellular Ca²⁺ influx on influenza virus. Neonatal pig tracheal epithelial (NPTr) cells (Ferrari et al., 2003) and 12-day-old porcine primary muscle cells (myotubes) were infected with 0.5 MOI pdm H1N1 and 2.0 MOI USSR H1N1 virus (respectively) for 2 h, rinsed with PBS and subsequently kept in different [Ca²⁺] (calcium concentration) of culture media (100 mg/mL; 200 mg/mL; or 300 mg/mL) for 24 h. FIGS. 1A and 1B show that raising extracellular [Ca²⁺] in the culture media of influenza virus infected cells resulted in significantly reduced production of progeny virus.

Cell viability of each cell type (CellTiter-Glo luminescent assay, Promega), determined at 24 h of incubation, was unaffected by different [Ca²⁺] (100 mg/mL; 200 mg/mL; or 300 mg/mL), indicating that different Ca²⁺ concentrations had no adverse impact on cell viability (FIGS. 1C and 1D).

The results unexpectedly show that simply raising extracellular [Ca²⁺] in the culture media of influenza virus infected NPTr cells and porcine myotubes resulted in significantly reduced production of progeny virus.

### Example 3

This example shows that Ca²⁺-release activated-Ca²⁺ (CRAC) mediated store-operated Ca²⁺ entry (SOCE) in a wide range of cell types reduced influenza A virus production.

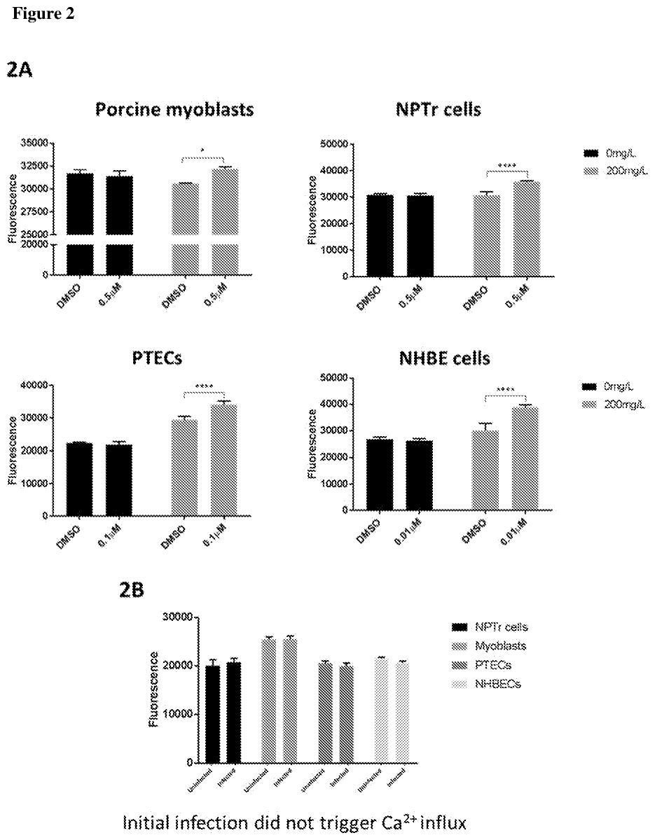

To assess the effect of extracellular Ca²⁺ influx on influenza virus, cells were transiently exposed to thapsigargin (TG) to impede the SERCA pump (Lytton et al., 1991) leading to SOCE (Hogan and Rao, 2015). Fluo-8 No Wash Calcium Assay Kit (Abcam) was used to determine intracellular Ca²⁺ accumulation. Porcine myoblasts, NPTr cells, primary pig tracheal epithelial cells (PTECs) and normal human bronchial epithelial (NHBE) cells were exposed to TG, at non-toxic concentrations (0.5 μM/0.1 μM/0.01 μM; see FIG. 2A), or to DMSO as control for 10 min in the presence of 0 mg/L or 200 mg/L extracellular calcium chloride in culture media after which fluorescence measurements were taken (FIG. 2A). Porcine myoblasts, NPTr cells, PTECs and NHBE cells were infected with USSR H1N1 virus at 2.0, 1.0, 1.0 and 1.0 MOI respectively for 15 min before intracellular Ca²⁺ fluorescence readings were taken (FIG. 2B). There was no significant Ca²⁺ influx detected during early virus infection. Significance determined by 2-way ANOVA, relative to corresponding DMSO treatment.

The results show that modest Ca²⁺ influx triggered by brief TG exposure (10 min) in several cell types (NPTr cells, primary porcinemyoblasts, PTECs and NHBE cells) was dependent on extracellular Ca²⁺, as determined by Fluo-8 Ca²⁺ assays (FIG. 2A). USSR H1N1 virus infection of NPTr cells, myoblasts, PTECs and NHBE cells for about 15 min duration, unlike the use of TG, did not elicit detectable Ca²⁺ influx (FIG. 2B).

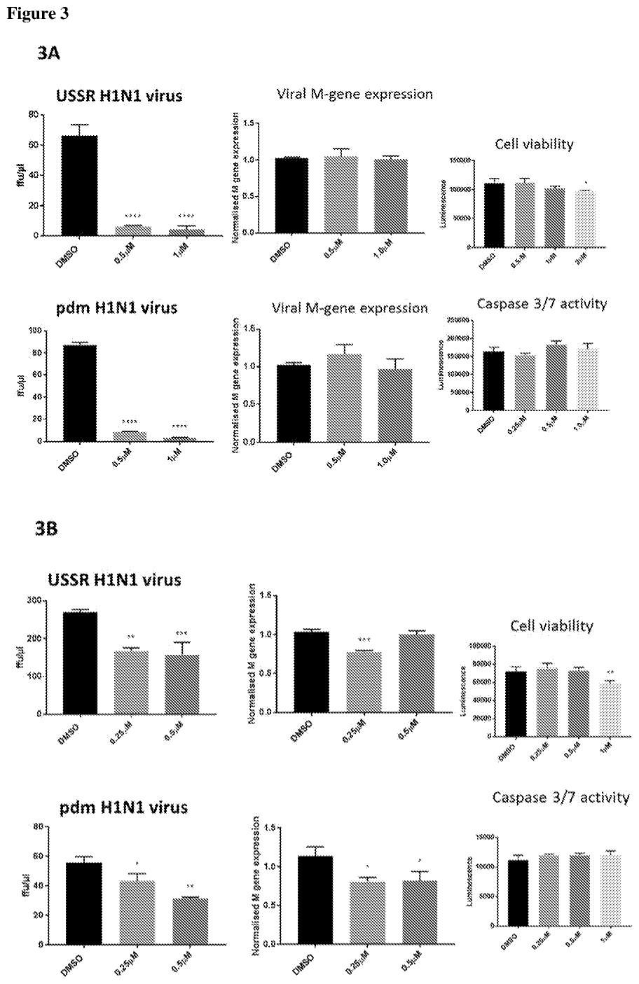

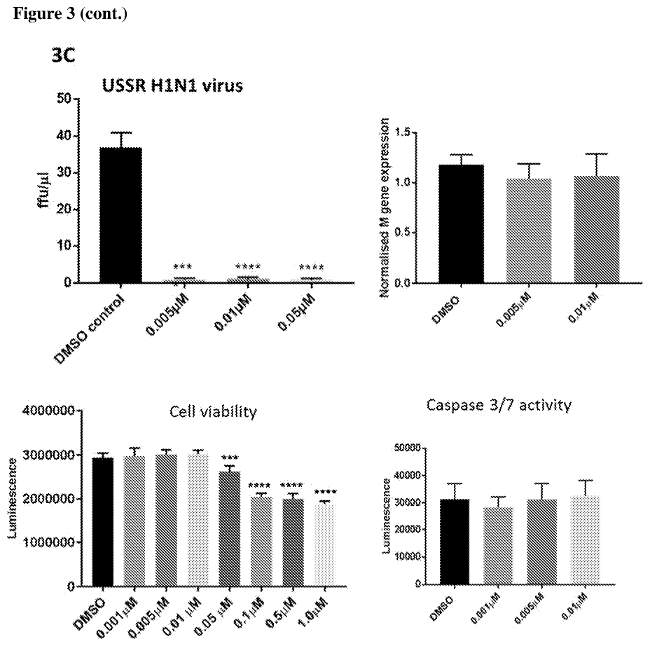

To determine the effect of TG priming on progeny virus output from infected cells, NPTr cells (FIG. 3A) and myoblasts (FIG. 3B) were incubated for 1 h, and NHBE cells (FIG. 3C) for 30 min with TG at concentrations as indicated. The cells were subsequently infected for 24 h with USSR H1N1 or pdm H1N1 virus at 0.5 MOI (based on 6 h focus forming assays). Spun supernatants were used to infect MDCK cells for 6 h in focus forming assays to determine progeny virus output. Viral M-gene expression, normalised to 18 s rRNA, derived from the comparative Ct method, showed no or little change from corresponding DMSO control. As shown in FIGS. 3A-3C, TG concentrations in all infection studies were non-toxic to cells based cell viability and apoptosis assays. Cell viability assays (CellTiter-Glo luminescent assay, Promega) and caspase 3/7 activity assays (Caspase-Glo 3/7 assay, Promega) were performed 24 h post-TG priming. Significance determined by one-way ANOVA in relation to corresponding DMSO control.

The results in FIG. 3 show that, importantly, NPTr cells (FIG. 3A), primary porcine myoblasts (FIG. 3B) and NHBE cells (FIG. 3C) primed with TG (for 30 min or 1 h) immediately before influenza virus infection consistently resulted in marked reduction in virus output. Virus inhibition was typically more pronounced in epithelial cells than myoblasts. The concentrations of TG used to prime each cell type had no cytotoxic effect on cell viability, based on host ATP production (CellTiter-Glo luminescent assay) and caspase 3/7 activity (Caspase-Glo 3/7 assay). Therefore, brief TG treatment of cells induces CRAC entry (SOCE) without detectable cytotoxicity and strongly inhibits influenza virus production.

### Example 4

This example assessed the sustained effect of TG, and the priming effect of TG before or during infection in reducing influenza virus production

The results described in example 3 suggest that among the different cell types evaluated, NHBE cells appeared most sensitive to TG priming; dramatic reduction in progeny virus output was achieved by priming with as little as 5 nM TG for 30 min (FIG. 3C).

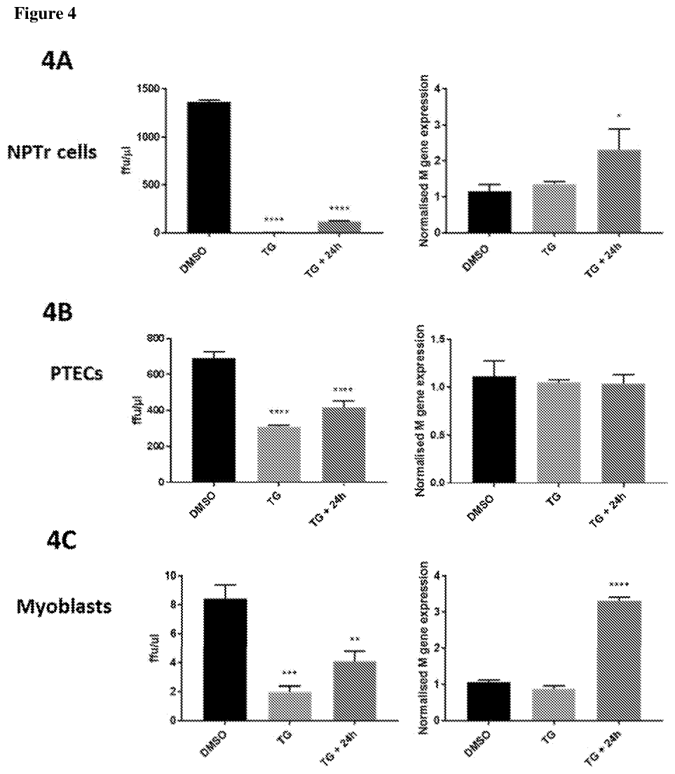

Experiments were conducted to assess the sustained antiviral effect of TG. NPTr cells were incubated with 0.5 μM TG for 30 min; PTECs were incubated with 0.1 μM TG for 30 min; myoblasts were incubated with 1.0 μM TG for 1 h. Cells were then rinsed with PBS and either immediately infected or further cultured for 24 h in normal media followed by infection (TG+24 h). NPTr cells and PTECs were infected with USSR H1N1 virus at 0.5 MOI, and myoblasts were infected with the same virus at 2.0 MOI. Spun supernatants from 24 h infected samples were used to infect MDCK cells for 6 hours in focus forming assays. Results are shown in FIG. 4. Significance determined by one-way ANOVA in relation to corresponding DMSO control.

The results in FIG. 4 show that duration of the antiviral state from non-toxic TG exposure was found to last for at least 24 h in NPTr cells, PTECs and myoblasts in that there was marked reduction in virus production from cells infected after an interval of 24 h from initial TG priming (FIG. 4A to 4C). Corresponding viral M-gene expression, normalised to 18 s rRNA, showed no reduction from TG treatment. There was some increased accumulation of M-gene RNA in NPTr cells and myoblasts from delayed infection (FIGS. 4A and 4C).

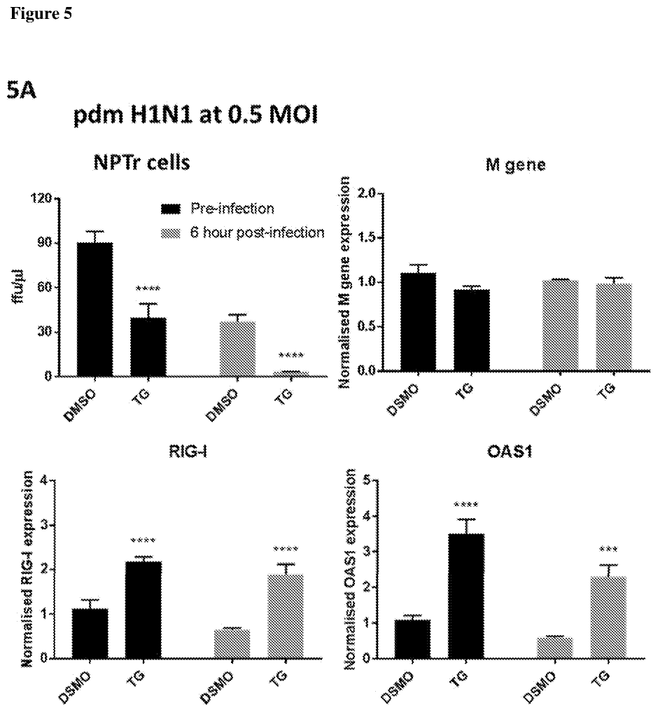

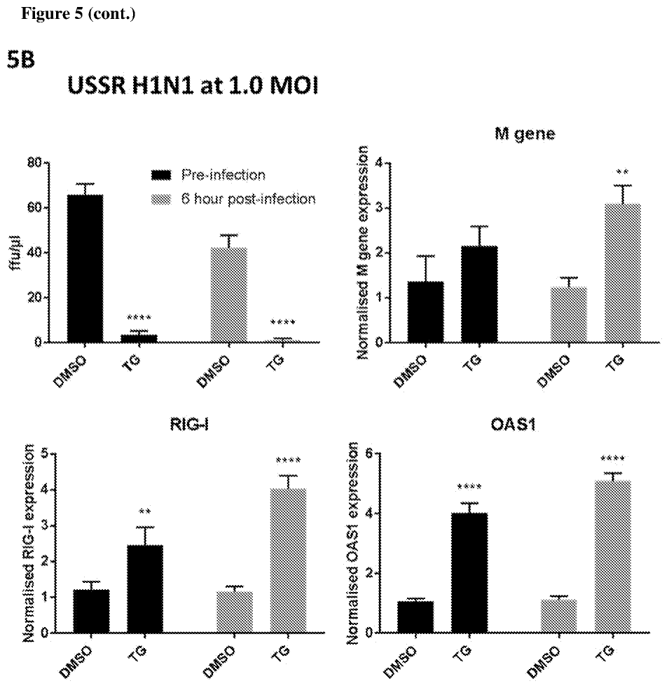

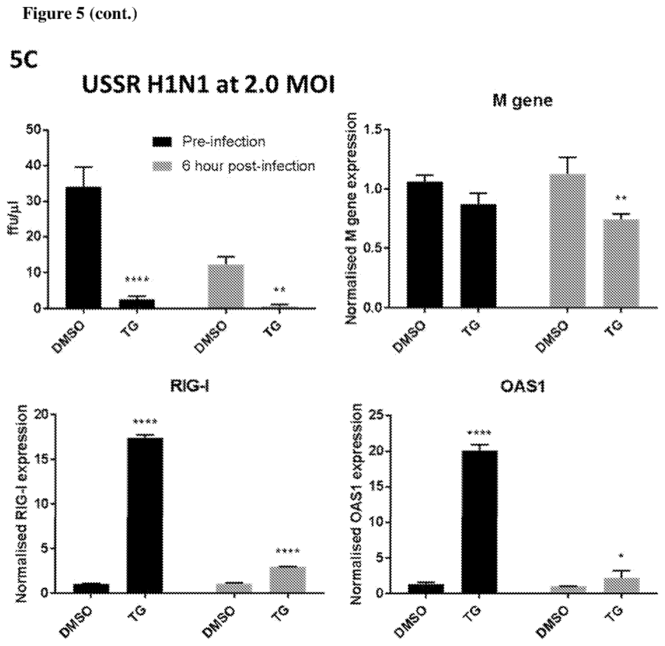

Further experiments were conducted to compare the antiviral activity of TG before or after an initial infection period of 6 h. Results are shown in FIG. 5. NPTr cells (FIG. 5A), NHBE cells (FIG. 5B) and porcine myoblasts (FIG. 5C) were incubated with TG for 30 min and immediately infected (TG pre-infection), or were first infected for 6 h followed by TG exposure for 30 min (TG post-infection). NPTr cells, NHBE cells and myoblasts were treated with TG at 0.5 μM, 0.01 μM and 0.5 μM respectively. NPTr cells, NHBE cells and myoblasts were infected with pdm H1N1 virus at 0.5 MOI, USSR H1N1 virus at 1.0 MOI and USSR H1N1 virus at 2.0 MOI respectively. Pre-infected TG primed cells, after initial 2 h of virus infection, were rinsed with PBS and cultured in fresh infection media overnight. Cells 6 h post-infection were treated with TG (30 min), rinsed with PBS and cultured in fresh infection media overnight. Progeny virus output was determined from spun supernatants. The results in FIG. 5 indicate that brief treatment of cells (NPTr cells, NHBE cells and myoblasts) with TG before or after an initial infection period of 6 h (virus replication cycle is only around 6 h) showed comparable reduction in virus production (FIG. 5A to 5C). This indicates that the antiviral states triggered by TG prior to infection, and during infection were similarly effective at virus reduction.

Variable viral M-gene expression, from corresponding infected cells normalised to 18 S rRNA, suggests post-transcriptional virus inhibition (FIG. 5). TG primed NPTr cells, NHBE cells and myoblasts, before and during infection, also showed strong induction of type I IFN associated genes (RIG-I and OAS1) in response to influenza virus infection (FIG. 5A to 5C).

### Example 5

This example describes experiments conducted to probe the origin of TG's antiviral activity. Without being bound by theory, the inventors believe that the results in this example indicate that TG has a role in elevating type I interferon (IFN) signalling in response to influenza virus infection.

Type I IFNs and their associated genes are essential for host defence against viruses (McNab et al., 2015). Priming of different cell types with TG, before or during infection, consistently increased the expression of type I IFN associated genes including RIG-I (retinoic acid-inducible gene 1, a cytoplasmic sensor of viral RNA) and OAS1 (2′-5′-oligoadenylate synthetase 1, an IFN-stimulated gene) in response to infection (FIG. 5A to 5C).

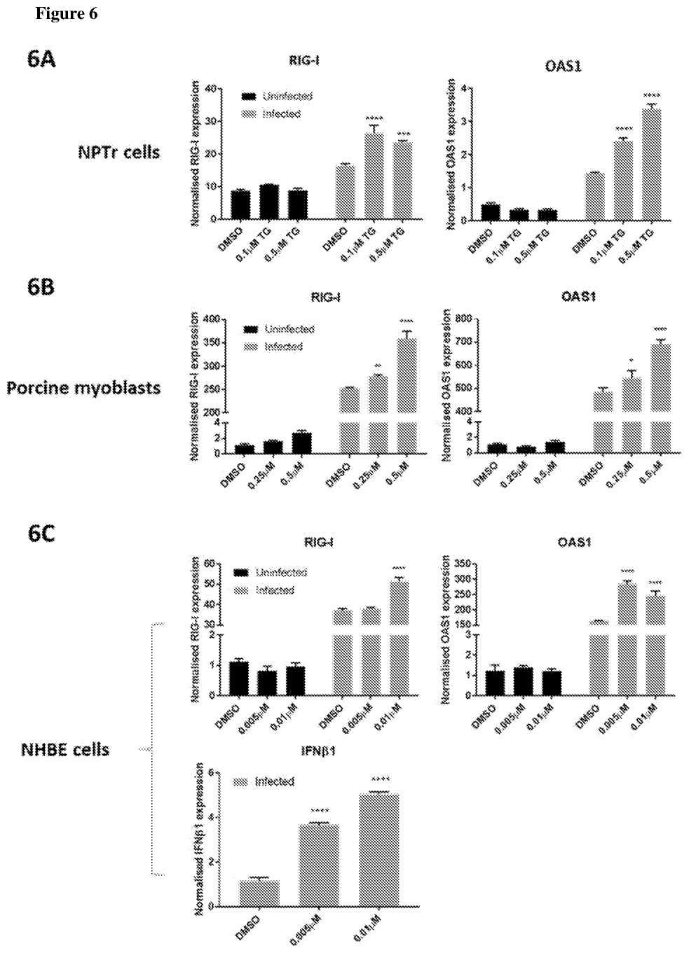

Experiments were conducted to assess whether the increased expression of RIG-I and OAS1 by TG priming occurs in a dose related manner. NPTr cells (FIG. 6A), porcine myoblasts (FIG. 6B) and NHBE cells (FIG. 6C) were incubated with indicated TG at non-toxic levels for 30 min, rinsed and respectively infected with USSR H1N1 virus at 0.5, 2.0 and 0.5 MOI for 24 h. IFNβ RNA in uninfected NHBE cells was below the threshold of detection but was up-regulated in a TG dose related manner during infection, as with OAS1 and RIG-I. Gene expression, normalised to 18 s rRNA, was based on the comparative Ct method relative to corresponding uninfected DMSO control. Significance is relative to corresponding DMSO control.

Without being bound by theory, the inventors consider that elevated expression of type I IFN associated genes by TG treated cells in response to infection (FIGS. 5 and 6) could be of paracrine importance in recruiting neighbouring cells to adopt an antiviral state ahead of infection. Together, the results described above indicate that TG activated SOCE is a potent antiviral pathway that remains active for ≥24 h post-TG priming , is effective when triggered before or during influenza virus infection, and mounts a clear type I IFN associated response to infection.

### Example 6

This example describes further experiments conducted to probe the origin of TG's antiviral activity. Without being bound by theory, the inventors believe that the results in this example indicate that TG has a role in interfering with influenza virus post-transcriptionally.

A feature of TG inhibition of virus production was the absence of consistent reduction in corresponding viral M-gene RNA expression (FIG. 3 to 5) which suggests that virus inhibition took place at post-transcription. The occasional increase in viral M-gene expression detected in TG-primed cells, such as in NPTr cells and myoblasts (FIG. 4), could represent accumulated backlog of incompletely processed viral RNA.

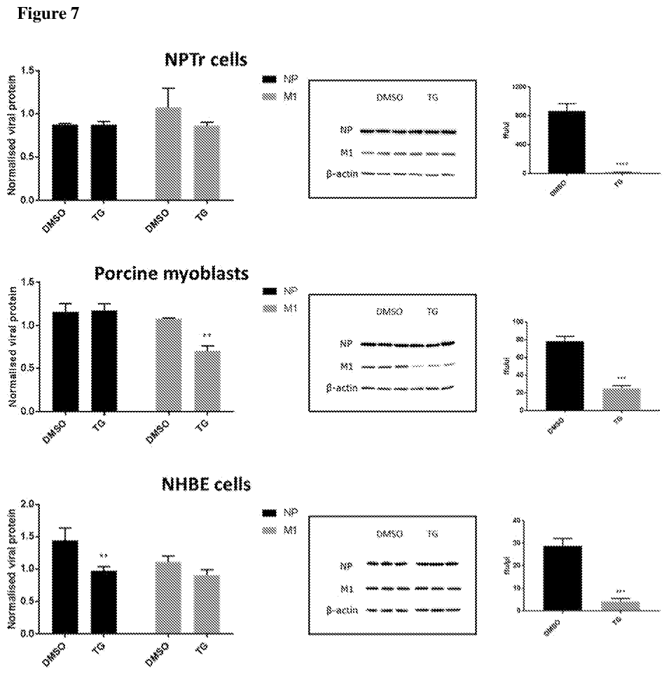

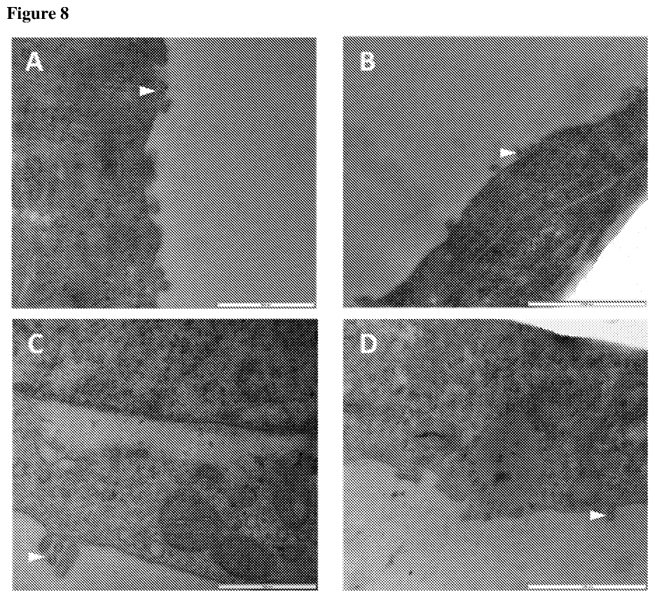

Experiments were conducted to probe this hypothesis. NPTr cells (FIG. 7A) and porcine myoblasts (FIG. 7B) were exposed to 0.5 μM TG, and NHBE cells (FIG. 7C) exposed to 0.005 μM TG for 30 min followed by 2 h of USSR H1N1 virus infection at 0.5, 2.0 and 1.0 MOI respectively. Cells were then rinsed with PBS and cultured for 24 h. Viral NP and M-gene protein, detected by Western blotting, were normalised to β-actin. Spun supernatants were used to infect MDCK cells for 6 h in focus forming assays. Significance determined by two-way ANOVA is relative to corresponding DMSO control. Western blotting results demonstrated no or only small reduction in viral NP and M1 protein in TG-primed infected cells (FIG. 7A to 7C) suggesting that virus disruption took place at the protein level, possibly affecting viral protein processing or assembly, which could involve a host mechanism distinct from the IFN associated response. Furthermore, NPTr cells primed with TG prior to infection did not appear to show abnormal viral morphology of budding influenza viruses suggesting that TG could have acted on reducing virus assembly (FIG. 8).

### Example 7

This example describes further experiments conducted to probe the origin of TG's antiviral activity. Without being bound by theory, the inventors believe that the results in this Example indicate that ER stress also contributed in part to TG mediated virus reduction.

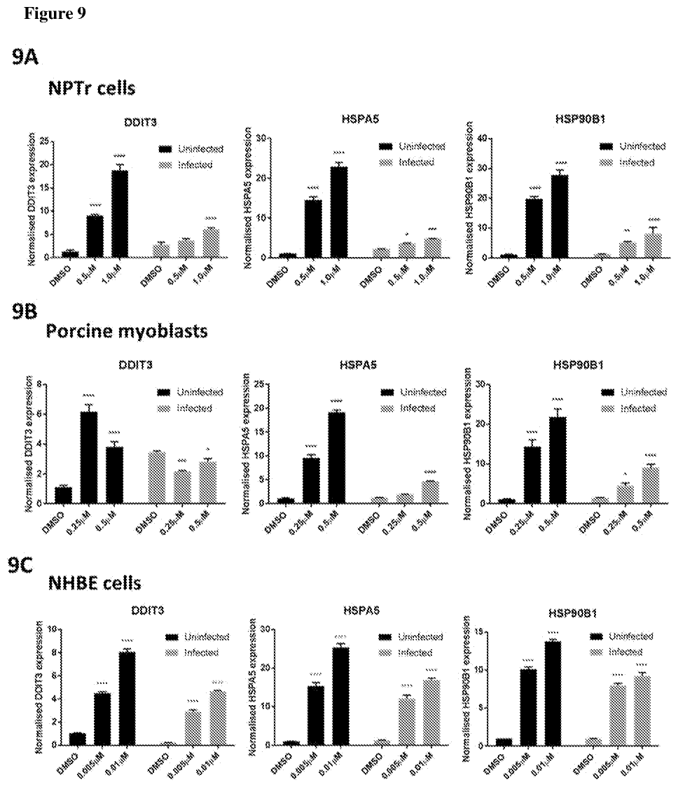

TG-induced CRAC influx is believed to be the culmination of three signalling events: (1) ER Ca²⁺ store depletion, followed by (2) ER stress and (3) extracellular Ca²⁺ entry through activated SOCE. ER stress-induced unfolded protein response (UPR) (Krebs et al., 2015) could interfere with viral protein translation and promote host innate immune response (Janssens et al., 2014; Lencer et al., 2015; Silva et al., 2007). Experiments were therefore conducted to assess whether priming cells with non-toxic doses of TG induced ER stress in a dose dependent response. NPTr cells (FIG. 9A), porcine myoblasts (FIG. 9B) and NHBE cells (FIG. 9C) were primed with TG as indicated for 30 min and then infected with USSR H1N1 virus at 0.5, 2.0 and 0.5 MOI respectively for 24 h. Expression of ER stress genes (DDIT3/Chop, HSPA5/Grp78/BIP, and HSP90B1/Grp94/Gp96) was normalised to 18 S rRNA. Significance, determined by one-way ANOVA within infected or uninfected treatments, is in relation to corresponding DMSO control.

The results show that TG applied at non-toxic levels consistently elevated ER stress associated genes (DDIT3, HSPA5 and HSP90B1) in different cell types (NPTr cells, porcine myoblasts and NHBE cells) in a dose-dependent manner (FIG. 9A to 9C). Interestingly, influenza virus infection attenuated the expression of ER stress genes in TG-primed cells (FIG. 9A to 9C) which might be viral mediated to promote viral protein processing.

### Example 8

This example describes comparative experiments demonstrating that inducing ER stress alone is not sufficient to fully account for the antiviral activity demonstrated by SOCE facilitators such as TG in accordance with the present invention.

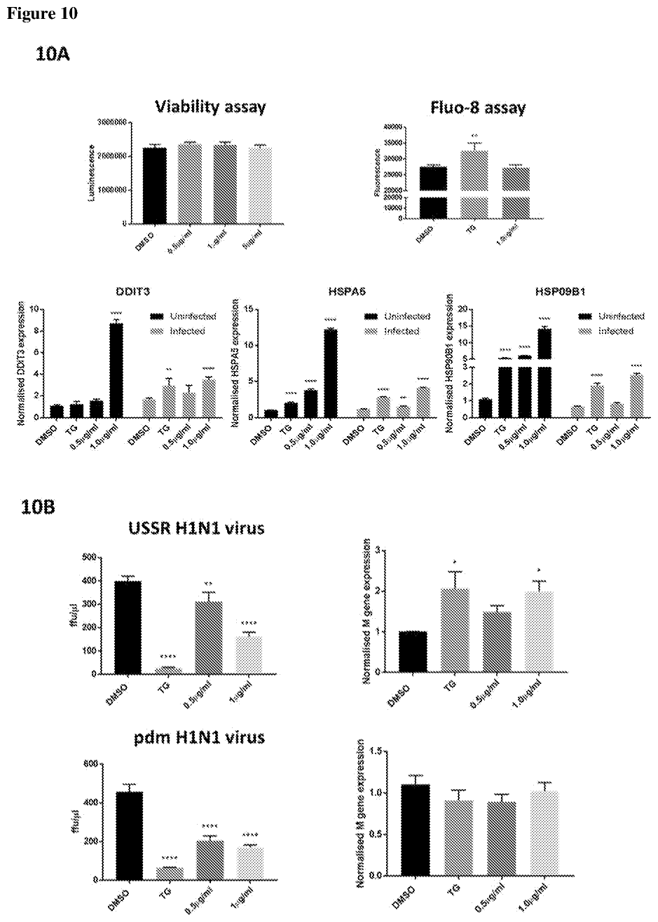

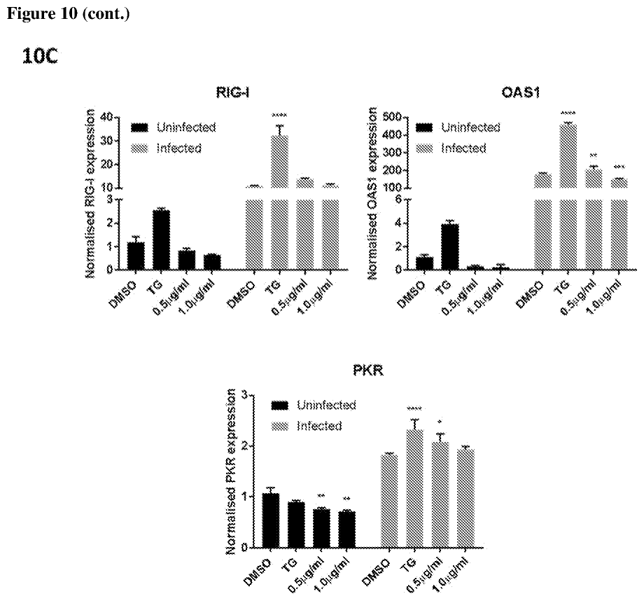

Tunicamycin, a glycosylation inhibitor, is often used as an inducer of ER stress (Oslowski and Urano, 2011; Michelangeli et al., 1995). Experiments were conducted to examine the role of ER stress per se on influenza virus production. NPTr cells were incubated with 0.5 μg/ml or 1.0 μg/ml tunicamycin for 30 min, rinsed with PBS, and infected with USSR H1N1 or pdm H1N1 virus at 0.5 MOI for 24 h. TG exposure, at 0.5 μM for 30 min prior to infection, served as a positive control (FIG. 10A to 10C). Expression of ER stress marker genes, DDIT3, HSPA5 and HSP09B1 (FIG. 10A), viral M-gene (FIG. 10B) and type I IFN associated genes (RIG-I, OAS1 and PKR) (FIG. 10C) was normalised to 18 s rRNA, based on the comparative Ct method. Spun supernatants were used to infect MDCK cells for 6 hours in focus forming assays (FIG. 10B). Significance determined by one-way ANOVA in relation to corresponding DMSO control.

NPTr cells were primed for 30 min before infection with non-toxic doses of tunicamycin that did not affect cell viability nor extracellular Ca²⁺ influx (FIG. 10A). The induction of ER stress associated genes (DDIT3, HSPA5 and HSP90B1) by tunicamycin (at 1.0 μg/ml) was about 3 to 8.2 fold higher than by TG (at 0.5 μM) (FIG. 10A). As with TG (as in FIG. 9), priming with tunicamycin attenuated the expression of ER stress genes during influenza virus infection (FIG. 10A). Importantly, tunicamycin primed cells only slightly reduced virus production without reduction in viral M-gene expression (FIG. 10B); virus production was 2.8 and 7.5 times higher with pdm H1N1 and USSR H1N1 virus respectively than from correspondingly infected TG primed cells (FIG. 10B). Furthermore, unlike the use of TG, tunicamycin conferred little change in the expression of type I IFN associated genes (RIG-I, OAS1 and PKR) in response to infection (FIG. 10C). Therefore, ER stress induced by TG appears to partially contribute to the overall reduction in virus production, but ER stress alone is insufficient to confer the full beneficial results observed for TG.

### Example 9

This example describes further experiments conducted to probe the origin of TG's antiviral activity. Without being bound by theory, the inventors believe that the results in this example indicate that facilitation of SOCE function (here by the over-expression of structural and positive regulatory SOCE genes) inhibits virus production.

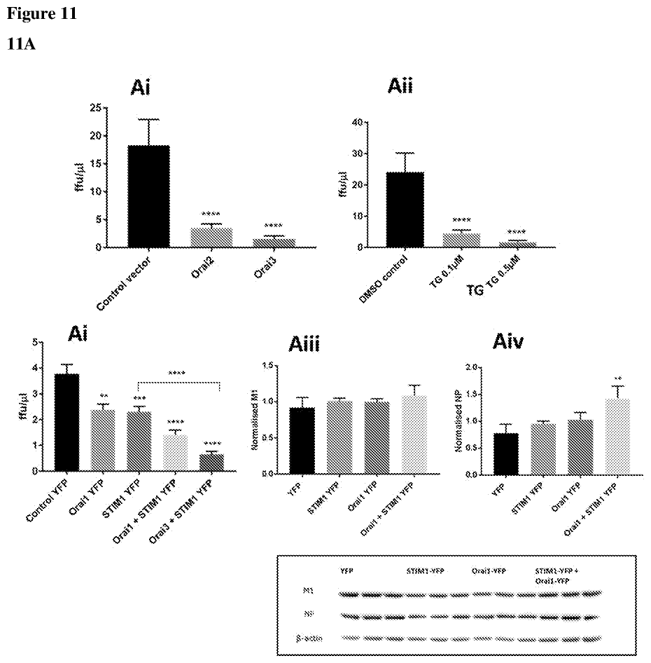

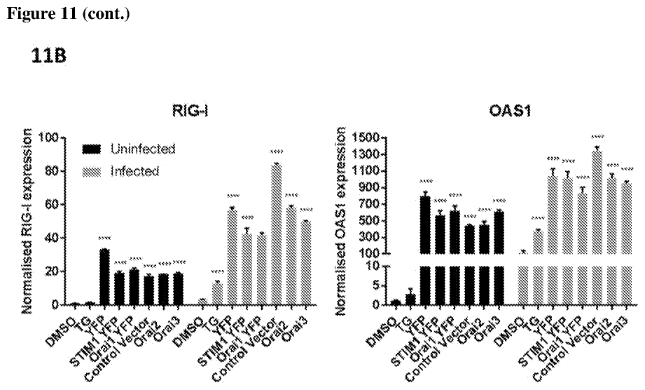

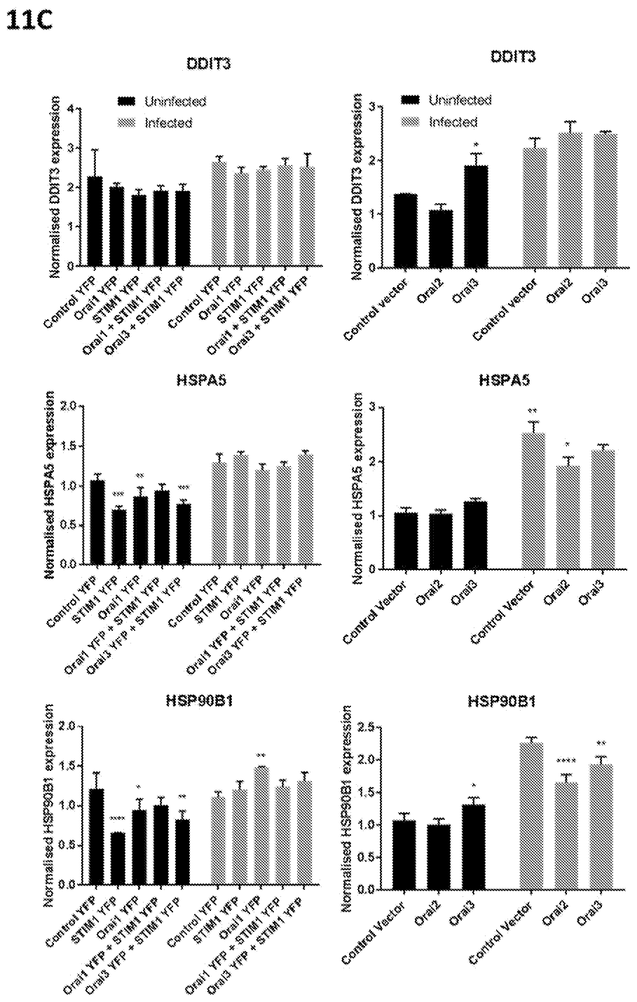

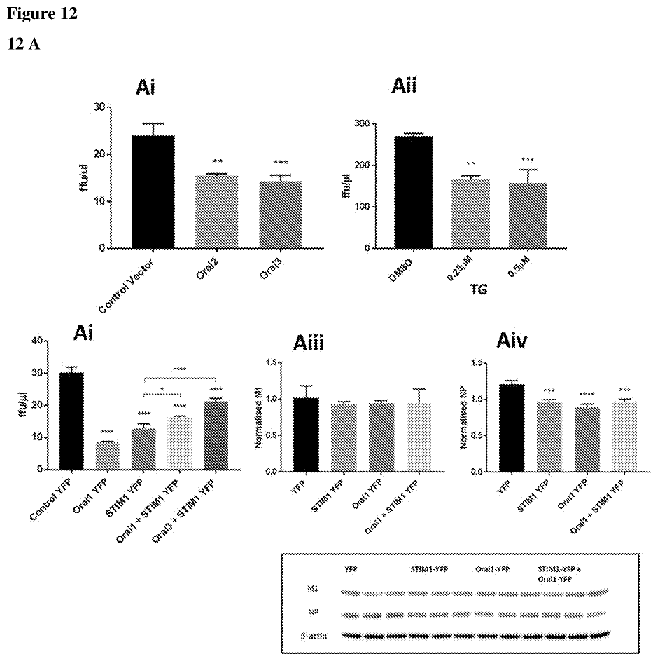

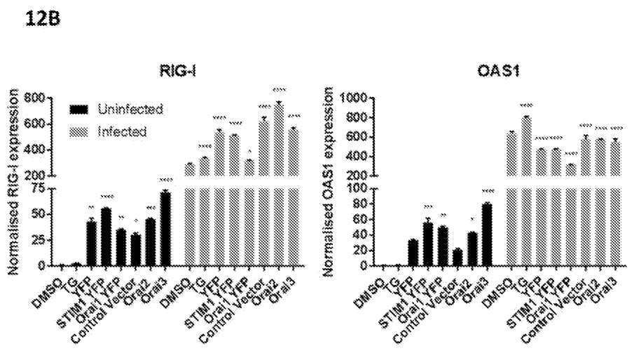

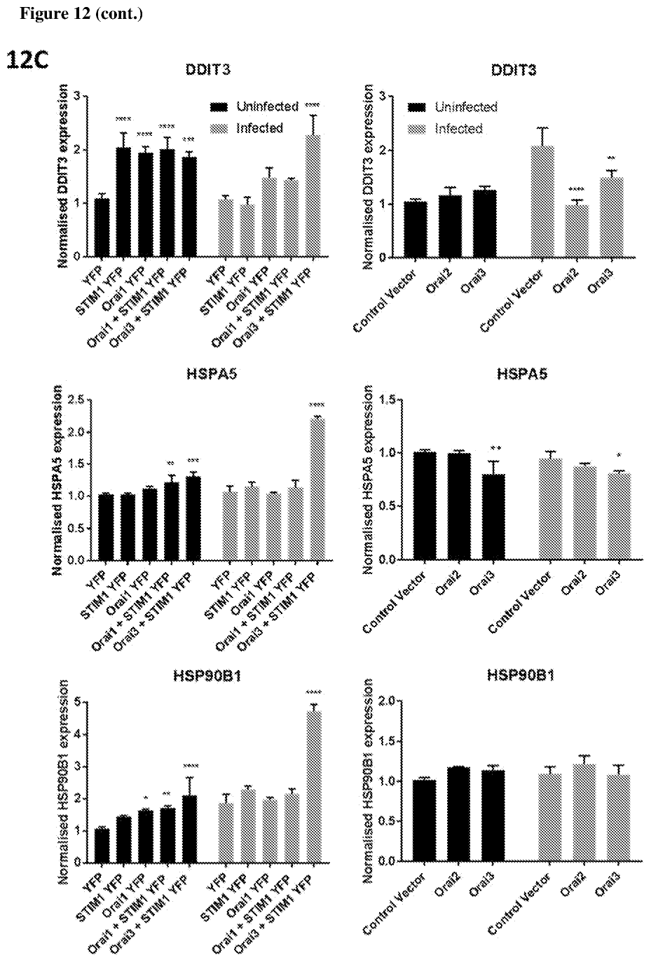

To further investigate the role of SOCE in influenza virus inhibition, structural members of SOCE, STIM1 and Orai1 to 3 isoforms, were over-expressed in NPTr cells (FIG. 11A to 11C) and primary porcine myoblasts (FIG. 12A to 12C) prior to infection.

NPTr cells, transiently transfected with the indicated plasmids for 2 days, were infected with USSR H1N1 virus at 0.5 MOI for 24 h. Spun supernatants were used in focus forming assays on MDCK cells infected for 6 h and immunostained for viral NP positive cells (FIG. 11Ai). Reduction in virus output was comparable to the use of TG (FIG. 11Aii), and expression of viral M1 protein and NP was unaffected by over-expression of SOCE genes (FIGS. 11Aiii and 11Aiv respectively). Expression of type I IFN associated genes (RIG-I and OAS1) (FIG. 11A), and ER stress related genes (DDIT3, HSPA5 and HSP90B1) (FIG. 11C), based on the comparative Ct method, was normalised to 18 S rRNA. Over-expression of STIM1 and Orai isoforms in NPTr cells slightly reduced the expression of type I IFN associated genes in response to infection FIG. 11B).Significance determined by one-way ANOVA in relation to corresponding DMSO control or vector control. YFP=yellow fluorescent protein. Graphs (FIG. 11) are representative of 3 experimental repeats.

In further experiments, porcine myoblasts, transiently transfected with the same indicated plasmids for 2 days, were infected with USSR H1N1 virus at 0.5 MOI for 24 h. Spun supernatants were used in focus forming assays on MDCK cells infected for 6 h and immunostained for viral NP positive cells (FIG. 12Ai). Reduction in virus output was comparable to the use of TG (FIG. 12Aii), and expression of viral M1 protein and NP was unaffected by over-expression of SOCE genes (FIG. 12Aiii and Aiv respectively). Expression of type I IFN associated genes (RIG-I and OAS1) (FIG. 12B), and ER stress related genes (DDIT3, HSPA5 and HSP90B1) (FIG. 12C), based on the comparative Ct method, was normalised to 18 S rRNA. Over-expression of STIM1 and Orai isoforms in myoblasts had little effect on the expression of type I IFN associated genes in response to infection (FIG. 12B). Significance determined by one-way ANOVA in relation to corresponding DMSO control or vector control. YFP=yellow fluorescent protein. Graphs (FIG. 12) are representative of 3 experimental repeats.

Over-expression of each SOCE member in NPTr cells led to reduced USSR H1N1 virus output of which Orai2 (81%) and Orai3 (92%) conferred the most virus reduction (STIM1 gave 39% reduction and Orai1 37%) (FIG. 11Ai). Correspondingly infected NPTr cells, pre-primed with 0.1 μM and 0.5 μM TG for 30 min, resulted in 82% and 93% virus reduction respectively (FIG. 11Aii). Given that transfection efficiency is intrinsically variable between experiments and between different sized constructs, the level of virus reduction mediated by individual SOCE members alone was comparable to the reduction achieved by TG priming. There was, however, no clear synergy in virus reduction from selected co-transfections of STIM1 and Orai1/Orai3 (FIG. 11Ai). Just like the use of TG (FIG. 7), over-expression of Orai1 and STIM1 did not affect production of viral M1 protein or NP indicating post-translational virus inhibition (FIGS. 11Aiii and 11Aiv). There was some reduction in the expression of type I IFN associated genes (RIG-I and OAS1) from SOCE transfected cells in response to virus infection; however, the high basal expression of type I IFN associated genes induced by transfection before virus infection could have accounted for the muted IFN response (FIG. 11B). Furthermore, over-expression of SOCE members had little effect on ER stress associated genes (DDIT3, HSPA5 and HSP90B1); typically there was less than one-fold difference in expression between each SOCE gene and corresponding control (FIG. 11C). The same over-expression study performed on primary myoblasts yielded virtually the same findings as those of NPTr cells: reduced virus output but no effect on viral M-gene protein or NP expression (FIG. 12A), no consistent effect on expression of IFN associated genes (RIG-I and OAS1) (FIG. 12B), and little or no effect on ER stress associated genes (FIG. 12C).

STIM-activating enhancer (STIMATE) and Ca²⁺ release activated channel regulator 2 A (CRACR2A) are positive regulators of SOCE. STIMATE encoded by TMEM110 is a multi-transmembrane ER protein that interacts with STIM1 (Jing et al., 2015; Quintana et al., 2015). CRACR2A is a Ca²⁺ sensor located in the cytoplasm that facilitates translocation and clustering with Orai1 and STIM1 to form a ternary complex (Lopez et al., 2016; Srikanth et al., 2010).

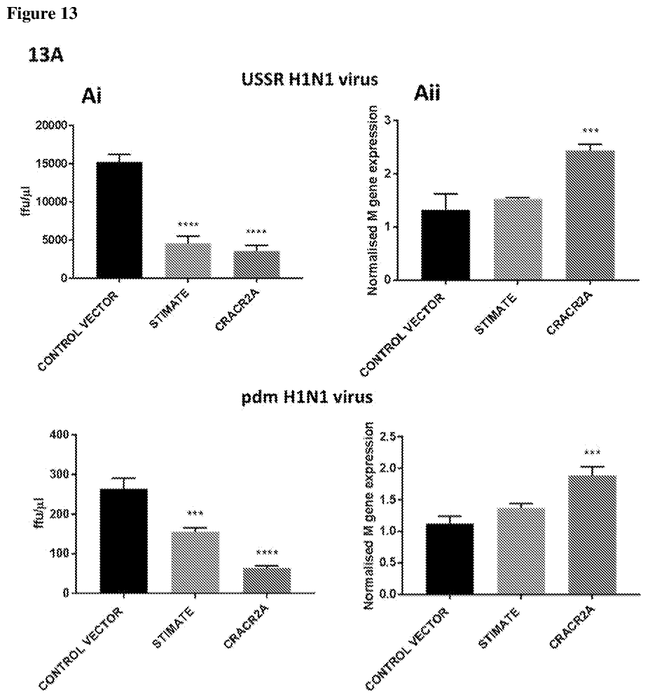

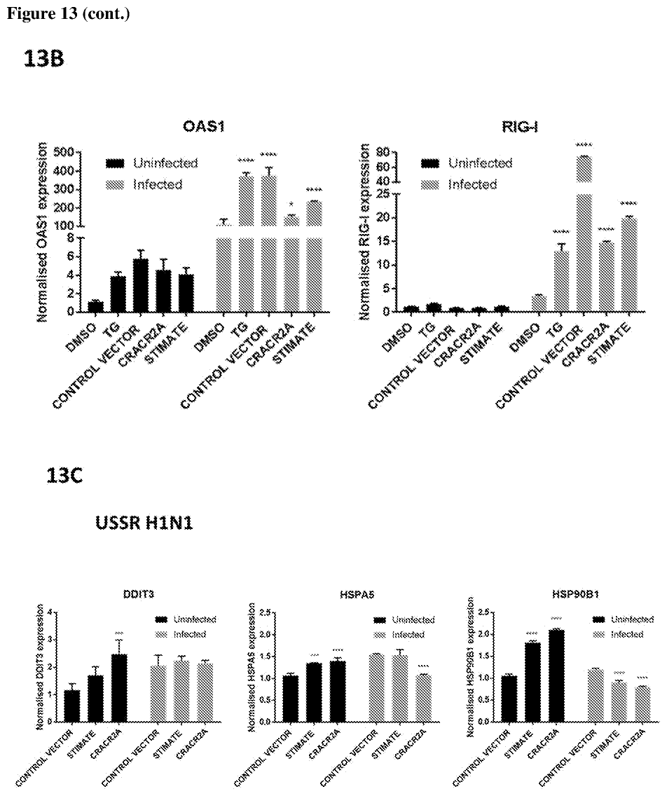

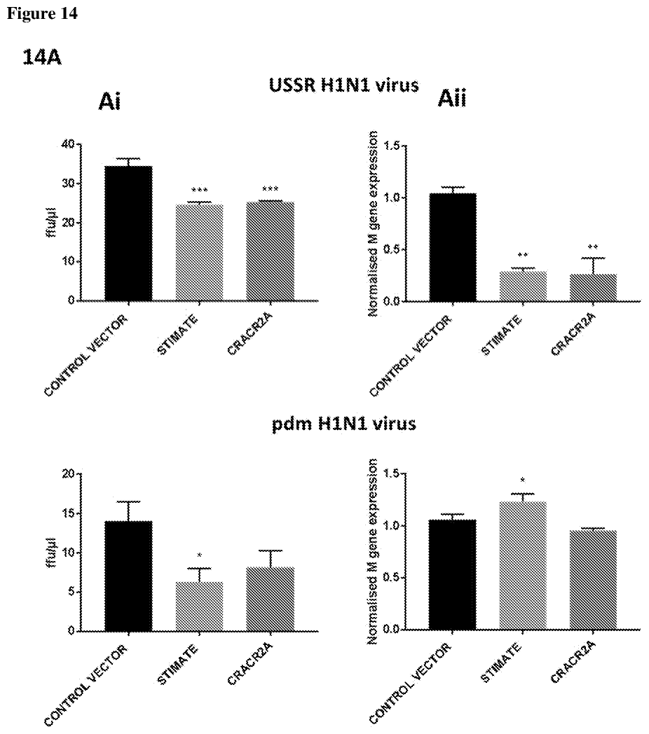

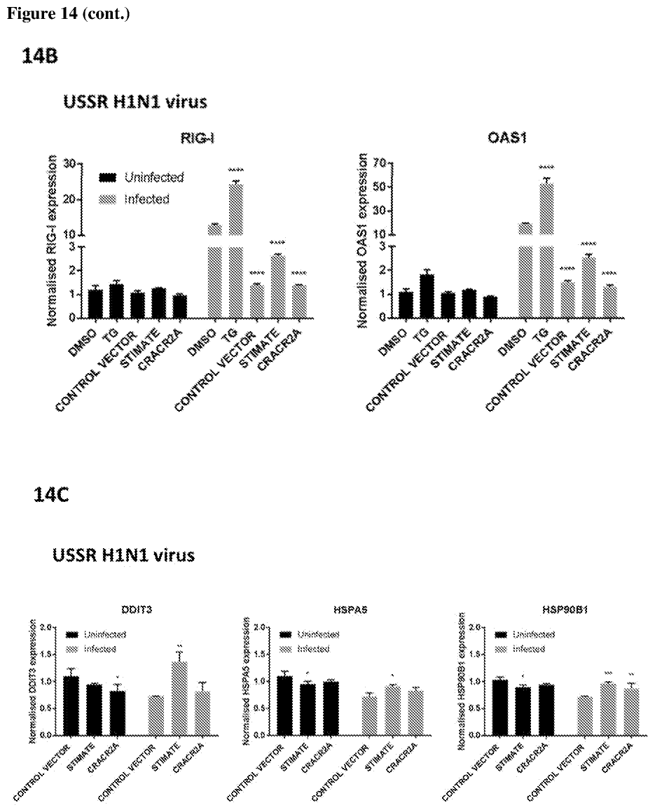

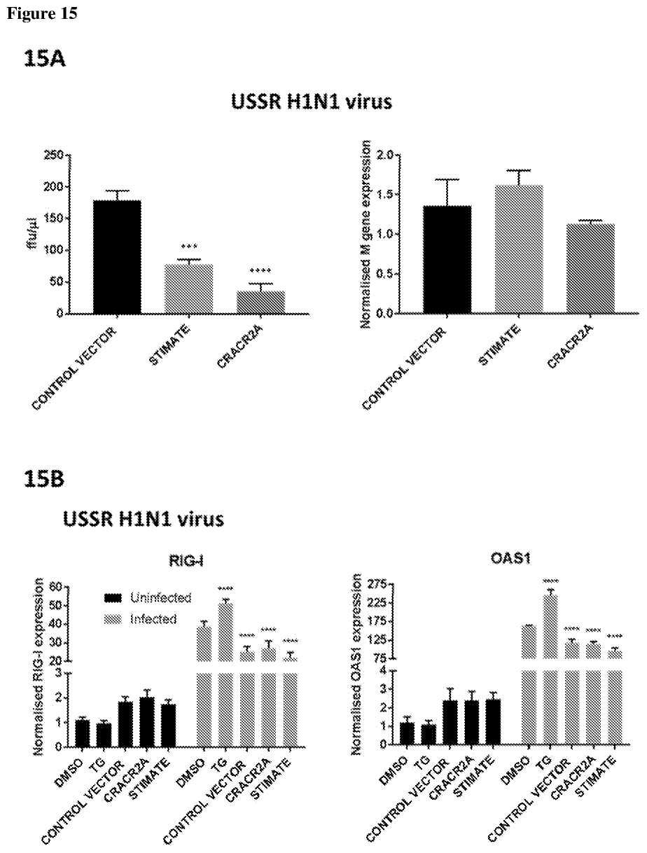

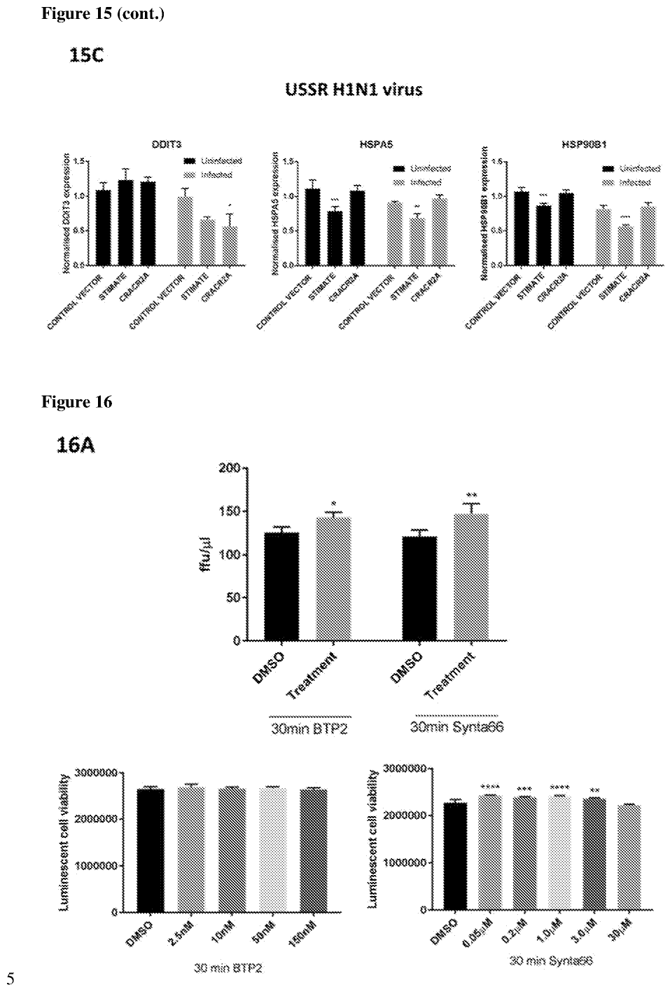

Accordingly, in still further experiments, NPTr cells, stably transfected with indicated plasmids to over-express CRAC2RA and STIMATE, were infected with USSR H1N1 at 0.5 MOI or pdm H1N1 virus at 1.0 MOI for 24 h. Spun supernatants were used to infect MDCK cells for 6 h in focus forming assays (FIG. 13A). Expression of viral M gene (FIG. 13A), IFN associated genes (FIG. 13B), and ER stress associated genes (FIG. 13C) was normalised to 18 S rRNA based on the comparative Ct method. Analogous experiments were conducted using porcine myoblasts (FIG. 14) and NHBE cells (FIG. 15). Porcine myoblasts transfected with indicated plasmids for 2 days were infected with USSR H1N1 virus at 2.0 MOI for 24 h. Spun supernatants were used to infect MDCK cells for 6 h in focus forming assays (FIG. 14A). Expression of viral M gene (FIG. 14A), IFN associated genes (FIG. 14B), and ER stress associated genes (FIG. 14C) was normalised to 18 S rRNA based on the comparative Ct method. NHBE cells, transfected with indicated plasmids for two days, were infected with USSR H1N1 virus at 1.0 MOI for 24 h. Spun supernatants were used to infect MDCK cells for 6 h in focus forming assays (FIG. 15A). Expression of viral M1 gene (FIG. 15A), IFN associated genes (FIG. 15B), and ER stress associated genes (FIG. 15C) was normalised to 18 S rRNA based on the comparative Ct method. Significance determined by a one-way ANOVA relative to corresponding vector control or DMSO control.

NPTr cells stably transfected with STIMATE or CRACR2A conferred substantially reduced progeny virus (66.7% and 73.3% USSR virus reduction respectively, and 42.31% and 76.9% pdm virus reduction respectively) (FIG. 13Ai), but without reduction in viral M-gene expression (FIG. 13Aii); these virus reductions too were comparable to those obtained from the use of TG (FIG. 11Aii). Similar to the over-expression of STIM1 and Orai isoforms, there was reduced expression of type I IFN associated genes (RIG-I and OAS1) in response to virus infection compared with control vector (FIG. 13B). Since this finding was based on stable transfections, the inhibition of virus production by STIMATE or CRACR2A over-expression, as with the over-expression of Orai and STIM1, was likely to be mediated by a Ca²⁺-linked antiviral mechanism distinct from the type I IFN associated response elicited by TG. Over-expression of STIMATE and CRACR2A also had little effect, typically with less than one-fold difference from control, on the expression of ER stress associated genes (DDIT3, HSPA5 and HSP90B1); notably small increase was detected in uninfected cells (FIG. 13C). Similar over-expression studies performed on primary myoblasts and NHBE cells, based on transient transfections, provided virtually the same findings as those of NPTr cells: reduced virus output (FIGS. 14A and 15A), and little or no change in the expression of IFN associated genes (RIG-I and OAS1) post-infection (FIGS. 14B and 15B), and ER stress associated genes (DDIT3, HSPA5 and HSP90B1) pre-infection (FIGS. 14C and 15C). In summary, facilitation of SOCE alone by individual over-expression of SOCE members largely recapitulated the effect of TG in reducing virus production but, unlike TG, had only minor impact on the expression of IFN-associated and ER stress-related genes.

These experiments confirm the role of SOCE in the antiviral activity exhibited by SOCE facilitators such as TG.

### Example 10

This example describes further experiments conducted to probe the origin of TG's antiviral activity. Without being bound by theory, the inventors believe that the results in this example confirm that facilitation of SOCE function inhibits virus production, by showing that inhibition of SOCE function promoted virus production.

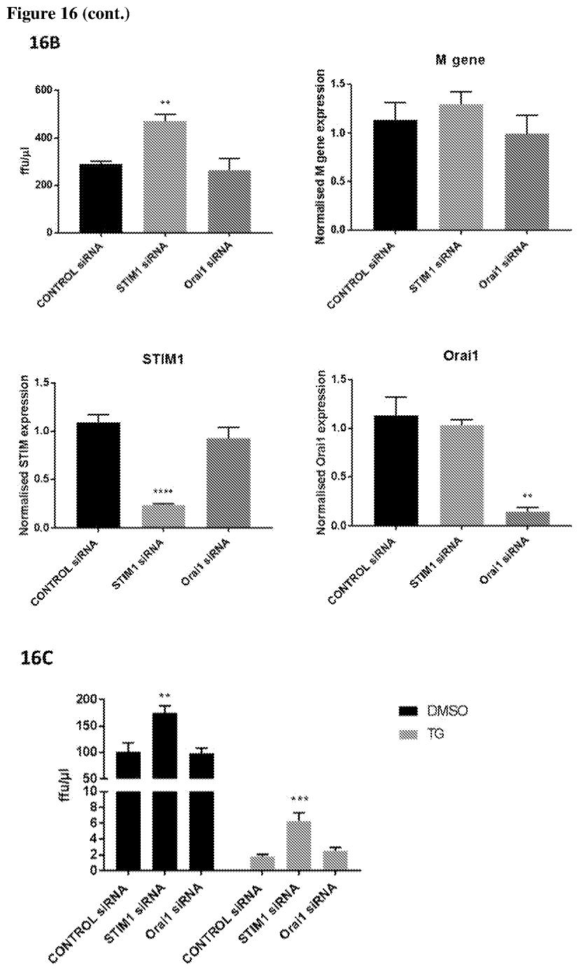

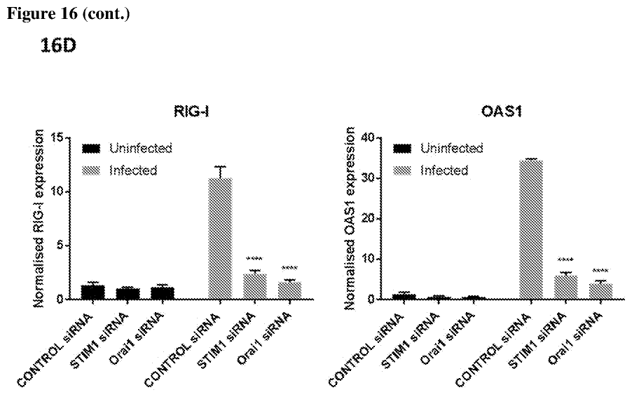

Further experiments were conducted to probe the original of TG's antiviral activity. NPTr cells were exposed to Orai inhibitors, 150 nM BTP2 and 5 μM Synta66 (FIG. 16A). Cells were primed with the respective Orai inhibitors for 30 min, rinsed with PBS and infected with USSR H1N1 virus at 0.5 MOI for 24 h after which spun supernatants were used to determine virus output by 6 h focus forming assays on MDCK cells. Non-toxic doses of BTP2 (150 nM) and Synta66 (5 μM) were used to prime NPTr cells; cells were exposed to each inhibitor for 30 min, rinsed with PBS, cultured overnight and assayed for cell viability (FIG. 16A). In further experiments, NHBE cells were separately transfected with the STIM1 and Orai1 siRNAs for 48 h were subsequently infected with USSR H1N1 at 1.0 MOI for 24 h (FIG. 16B). Spun supernatants were used to infect MDCK cells for 6 h in focus forming assays to determine progeny virus output. Total RNA was extracted from each sample to ascertain M gene, STIM1 and Orai1 gene expression, normalised to 18 S rRNA, based on the comparative Ct method. Effective knockdown of STIM1 and Orai1 was evident by reduced expression of respective RNA (FIG. 16B). STIM1 knockdown was accompanied by increased virus production but without significant effect on viral M gene expression (FIG. 16B). Knockdown of STIM1 but not Orai1 in NHBE cells reduced the inhibitory effect of TG in virus production (FIG. 16C). Knockdown of STIM1 and Orai1 also inhibited the up-regulation of RIG-I and OAS1 expression in response to infection (FIG. 16D). Significance determined by a one-way ANOVA in relation to corresponding control. The data in FIG. 16 indicate that inhibition of SOCE increased influenza virus output and that STIM1 knockdown in NHBE cells raised virus output.

Exposure of NPTr cells to chemical inhibitors of CRAC entry channel, BTP2 and Synta66 (Han et al., 2015), for 30 min prior to infection conferred a small increase in progeny virus release (FIG. 16A), further corroborating the role of Orai-STIM1 in the inhibition of influenza virus. To further demonstrate the anti-influenza role of SOCE, STIM1 and Orai1 were knocked down in NHBE cells for 48 h followed by infection with USSR H1N1 at 1.0 MOI for 24 h (FIG. 16b). STIM1 but not Orai1 knockdown raised virus output. The lack of virus increase in Orai1 knockdown could be due to functional redundancy displayed by other members of the Orai gene family. Like earlier findings, knockdowns had no significant effect on viral M gene expression. Reduced expression of STIM1 and Orai1, however, attenuated the induction of RIG-I and OAS1 expression in response to infection (FIG. 16D). Collectively, inhibition of SOCE function increased influenza virus production, and dampened the RIG-I and OAS1 response to infection in STIM1 and Orai1 knockdown NHBE cells underlining SOCE as a key anti-influenza innate immune process.

### Example 11

This example describes further experiments demonstrating the role of SOCE in inhibiting virus production.

Cyclopiazonic acid (CPA) is identified as a selective inhibitor of SERCA: inhibiting Ca²⁺ store refilling and enhancing Ca²⁺ entry into the cytosol (Uyama et al., 1993, Seidler et al., 1989). However, CPA is not efficient at SERCA inhibition hence relatively high concentrations are generally needed (Croisier et al., 2013).

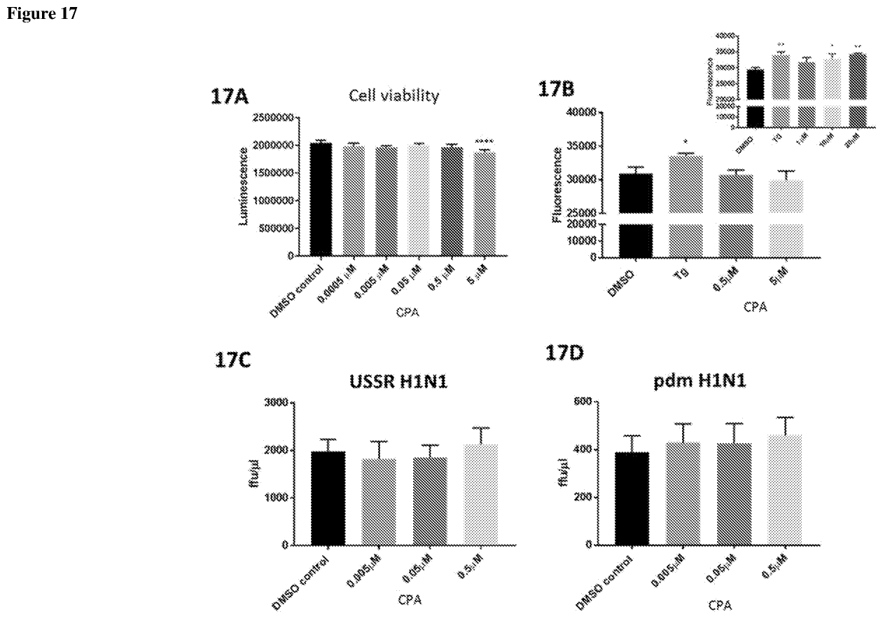

NPTr cells were exposed to different concentrations of CPA for 30 min, rinsed three times with PBS, incubated for 24 h and followed by luminescent cell viability assay (Celltiter-Glo, Promega) (FIG. 17A). At 5 μM CPA exposure, there was an 8.3% reduction in ATP production. Priming with CPA up to 5 μM did not elicit extracellular Ca²⁺ influx based on Fluo-8 Ca2+ assays. Only from 10 μM CPA was Ca²⁺ influx detected (FIG. 17B, inset). Cells were primed with up to 0.5 μM CPA as indicated for 30 min at indicated concentrations, rinsed three times with PBS and subjected to overnight infection with USSR H1N1 (FIG. 17C) and pdm H1N1 (FIG. 17D) virus each at 0.5 MOI. Spun supernatants were used in focus forming assays on MDCK cells infected for 6 h and immunostained for viral NP positive cells. There was no significant reduction in virus output from NPTr cells pre-treated with any of the indicated non-toxic doses of CPA (FIGS. 17C and 17D) supporting the need for SOCE (extracellular Ca²⁺ influx) to inhibit influenza virus production.

### Discussion of Examples 2 to 11

The inventors have shown for the first time that CRAC entry (via SOCE) is a potent innate immune defence against influenza A viruses. The inventors discovered that brief non-cytotoxic priming of a range of cell types, including primary NHBE cells, by SOCE facilitators such as TG strongly reduces virus production.

The inventors demonstrated that activated CRAC entry induced by SOCE facilitators in accordance with the invention is similarly effective at virus reduction whether it is activated before or during infection, and sustains resistance to infection for ≥24 h post-TG exposure.

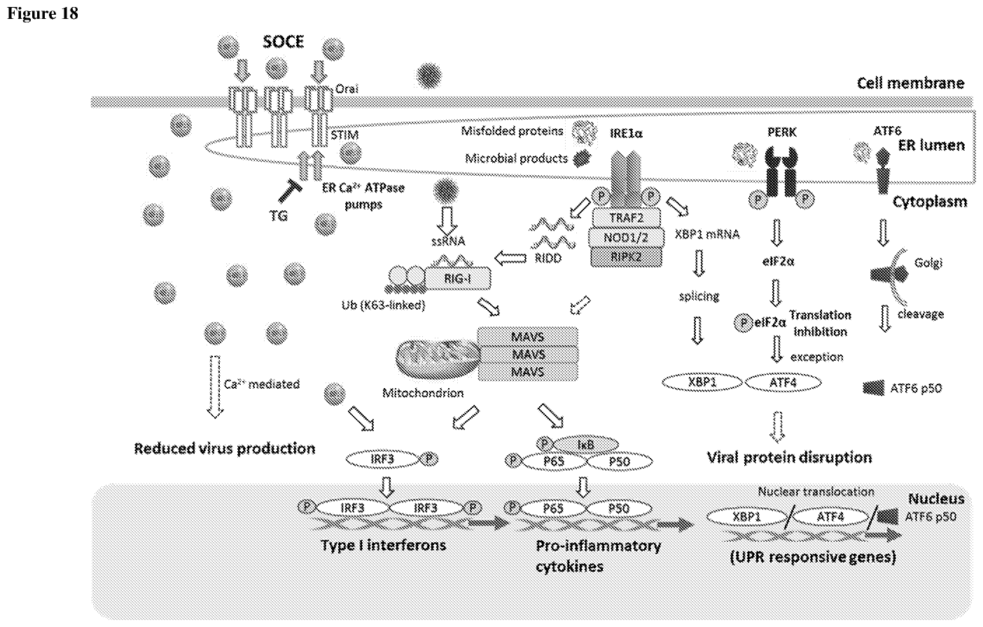

The inventors further showed that virus disruption took place post-transcriptionally at the protein level, possibly affecting viral protein processing or assembly. The antiviral effects of SOCE facilitators such as TG thus appear sustained and rapid in onset. Without being bound by theory, the inventors consider that, mechanistically, SOCE facilitators such as TG seem to operate at several levels to target the inhibition of influenza virus production (FIG. 18).

In this model, CRAC entry mediated by SOCE facilitators such as TG is accompanied by ER stress associated UPR (Krebs et al., 2015) that involves three major ER-transmembrane sensors: ATF6, PERK and IRE1. Upon activation, the precursor form of ATF6 translocates to the Golgi apparatus to be cleaved to release the active ATF6 p50 which is shuttled into the nucleus to transactivate UPR responsive genes, such as ER chaperons (Hassan et al., 2012; Lencer et al., 2015). Activated PERK phosphorylates eIF2α that results in the inhibition of global protein translation that includes the inhibition of influenza virus protein production (Landera-Bueno et al., 2017; Lencer et al., 2015). The activation of ATF6 and PERK can also lead to the activation of NF-κB and induction of cytokines (Janssens et al., 2014). Activated IRE1α, as a kinase as well as an endoribonuclease, appears to have a dual role as an ER stress sensor and a pathogen recognition receptor (PRR) of single stranded RNA generated by its own endoribonuclease action (Cho et al., 2013; Lencer et al., 2015). Activated IRE1α splices the XBP1 mRNA that results in XBP1 translation which in turn up-regulates the expression of ER chaperon and lipogenic genes. Misfolded proteins or microbial (bacterial) products may also activate IRE1α to generate single stranded RNA from host mRNA which induces RIG-I signalling that leads to NF-κB and IRF3 activation (Lencer et al., 2015). Furthermore, ER stress has been shown to recruit NOD1/2-TRAF2-RIPK2 complex to IRE1α leading to the activation of NF-κB that induces IL6 expression (Keestra-Gounder et al., 2016). NOD1 and NOD2 are traditionally regarded as cytosolic sensors of bacterial peptidoglycan fragments, but NOD2 can also function as a cytoplasmic viral PRR for viral ssRNA, including influenza A virus, by signalling through adaptor protein, MAVS adaptor, to trigger the activation of IRF3 and production of IFN-β (Sabbah et al., 2009). Therefore, activated IRE1α is a major site for the transduction of ER stress and innate immune signalling of RIG-I and NOD1/2.

In the experiments described above, the inventors showed that the CRAC entry-mediated inhibition of influenza virus production, triggered by brief non-toxic exposure to TG, appears to operate at several separate levels. Without being bound by theory, the inventors surmise that TG induced-ER stress would have an almost immediate effect in disrupting viral protein production/processing, and TG could have primed cells to subsequently mount a vigorous RIG-I-type I FN signalling response to influenza virus infection. Facilitation of SOCE alone (by over-expression of structural or positive regulatory SOCE members) was sufficient to induce a robust and seemingly IFN-independent antiviral state.

In probing the origin of the above effects, the inventors found that (a) TG-primed cells exhibited elevated type I IFN associated response to infection in a dose-related manner. Such an antiviral response would require de novo protein synthesis and may be a specific Ca²⁺ transduction effect of TG stimulation. The inventors also found that (b) the post-transcriptional inhibition of influenza virus was in part mediated by the induction of ER stress, as evidenced by the use of tunicamycin that did not affect Ca²⁺ influx but likely to have involved PERK activation that promptly inhibited viral protein production or processing (Landera-Bueno et al., 2017; Yan et al., 2002; Connor and Lyles, 2005). The inventors further found that (c) facilitation of SOCE alone (by over-expression of structural or positive regulatory SOCE members) was sufficient to induce a robust antiviral state. Over-expression of SOCE genes (structural and regulatory) did not appear to have any appreciable effect on ER stress. Conversely, knockdown of STIM1 and Orai1 (genetic inhibition of SOCE) promoted virus production, confirming the specificity of SOCE in the inhibition of influenza virus. Presently, it is not completely clear how Ca²⁺ influx via SOCE is transduced into post-transcriptional inhibition of influenza virus. The cross-talk between SOCE and the type I IFN associated response requires further investigation. The inventors found that TG primed cells elevated the expression of IFN associated genes in response to infection. Genetic inhibition of SOCE, conversely, attenuated expression of type I IFN related genes during infection. However, facilitation of SOCE by over-expression of SOCE members, whilst effective in the inhibition of virus production, had little effect on the expression of type I IFN associated genes in response to influenza virus infection. Without being bound by theory, the above indications are believed to support the role of SOCE facilitation in generating a robust antiviral response particularly against influenza viruses.

Without being bound by theory, the inventors believe that the magnitude and duration of SOCE triggered by SOCE facilitators such as TG are likely to be key in conferring host resistance to influenza virus. Induction of modest CRAC influx, as detected in Fluo-8 Ca²⁺ assays, may be all that is required to induce a potent antiviral state. Consistent with this thinking, facilitation of SOCE alone, by the over-expression of SOCE members, was shown to resist virus infection, presumably through small transient Ca²⁺ influx triggered during early infection. Furthermore, the lack of reduction in progeny virus following pre-treatment with CPA at doses that appear not to induce or facilitate Ca²⁺ influx is consistent with this view.

On its own, early influenza virus infection did not induce detectable extracellular Ca²⁺ entry in different cell types. By contrast, chronic surge in Ca²⁺ influx is damaging and can lead to apoptosis such as in rotavirus infection (Halasz et al., 2010; Hyser et al., 2013; Flourakis et al., 2010). Induction of extracellular Ca²⁺ influx during late stages (≥24 h) of highly pathogenic avian influenza H5N1 virus infection in duck embryonic fibroblasts was found to induce apoptosis thus facilitating virus propagation (Ueda et al., 2010). Furthermore, hemorrhagic fever viruses, at a late stage of virus replication, trigger SOCE which is necessary for virus budding (Han et al., 2015). TG-induced increase of cytosolic Ca²⁺ is primarily through extracellular Ca²⁺ influx (May et al., 2014). Influenza virus appears particularly susceptible to transient activation of CRAC entry.

Tunicamycin as an ER stress inducer inhibits protein glycosylation and palmitoylation; it increases Ca²⁺ influx across the plasma membrane (via SOCE) (Czyz et al., 2009; Zhu-Mauldin et al., 2017) and, in part, by ER Ca²⁺ store depletion (Buckley and Whorton, 1997; Czyz et al., 2009; Deniaud et al., 2008). The non-toxic doses of tunicamycin used in the present study induced ER stress but without detectable extracellular Ca²⁺ influx. Not surprisingly, influenza virus infection has been shown to induce ER stress (Roberson et al., 2012; Hrincius et al., 2015; Hassan et al., 2012). However, the inventors found that influenza virus infection also attenuated the ER stress response in cells primed with TG prior to infection. ER stress and influenza virus infection are known to transcriptionally activate P58IPK, an inhibitor of eIF2α kinases including PERK, PKR and GCN2, that reduces the phosphorylation of eIF2α thus, in a negative feedback, promoting protein translation and alleviating ER stress (Yan et al., 2002; Roobol et al., 2015).

At sufficiently high concentration, TG can be toxic to cells leading to apoptosis (Denmeade et al., 2003; Linford and Dorsa, 2002; Wang et al., 2014). Since it is often used to induce ER stress, through ER Ca²⁺ store depletion, it is no surprise that basic cellular functions, such as ATP production (indication of relative viability) and caspase activity (indication of apoptotic progression), can be adversely affected. In such studies, TG is typically applied at relatively high concentration (in μM range) (Tsalikis et al., 2016; Perry et al., 2012) and/or over an extended period (h to days) (Dombroski et al., 2010; Denmeade et al., 2003; Wang et al., 2014). However in virus experiments, the use of TG without proper monitoring of cell viability can complicate the interpretation of findings. Cells with compromised viability can appear morphologically normal but are less able to support full virus production. Therefore, it is necessary that in virus infections, where TG is included to induce ER stress or SOCE, the effect of cytotoxicity is ascertained. In our experiments, unlike some studies (Michelangeli et al., 1995; Fujioka et al., 2013) we explicitly applied TG in the non-toxic range for each cell type, such that it had no detectable cytotoxicity (based on luminescence ATP cell viability and caspase 3/7 assays). By contrast, previous reports of the effect of TG on viral replication have typically applied the TG at potentially cytotoxic levels and/or for extended duration (Michelangeli et al., 1995) such that any observed decrease in infectivity can be assigned to apoptosis and/or cytotoxicity, rather than inhibition of viral replication and/or infectivity. This is consistent with more recent reports which have shown that an increase in cytosolic Ca²⁺, e.g. mediated by the viral non-structural protein 4 (NSP4), is necessary for rotavirus replication (Hyser et al., 2013). To combat pathogenic influenza virus infection, it is strategically more effective to strengthen host resistance than to directly target the virus which can easily mutate to circumvent the antiviral drug. The identification of CRAC entry via SOCE as a potent innate immune defence against influenza virus infection and the exemplification of TG as an SOCE agonist have opened up the possibility of new therapeutics that target the SOCE pathway to blunt the severity and transmission of influenza virus infection.

### Example 12

This example shows that other sesquiterpene lactones also have antiviral effects.

Like TG, artemisinin is a sesquiterpene lactone. The inventors conducted experiments to demonstrate that cells previously primed with artemisinin produced less progeny virus when infected with influenza virus.

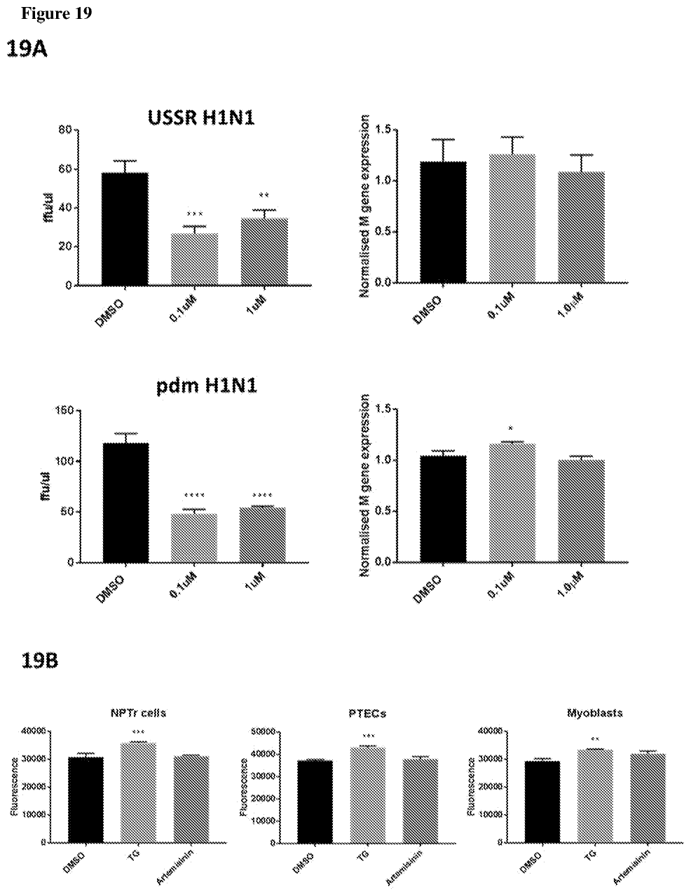

NPTr cells, incubated with 0.1 μM and 1.0 μM artemisinin for 30 min were subsequently infected with USSR H1N1 or pandemic H1N1 virus at 0.5 MOI for 24 h. Spun supernatants were used to infect MDCK cells for 6 h in focus forming assays. Significance determined by one way ANOVA, comparing to corresponding DMSO control. Data are shown in FIG. 19A which demonstrates that, like TG, artemisinin reduces progeny viral output from cells infected by either USSR H1N1 or pandemic H1N1 viral strains without significant alteration in viral M-gene expression (FIG. 19A).

The inventors conducted further experiments to determine the effect of artemisinin on extracellular Ca²⁺ influx. Fluo-8 fluorescence intensity was measured in NPTr cells, PTECs and porcine myoblasts pre-incubated with 1.0 μM artemisinin or TG at 0.5 μM, 0.1 μM and 0.5 μM respectively, for 10 min. Significance was determined by one way ANOVA, relative to the corresponding DMSO control. Results are shown in FIG. 19B.

### Example 13

This example shows that still other sesquiterpenes and sesquiterpene lactones also have antiviral effects.

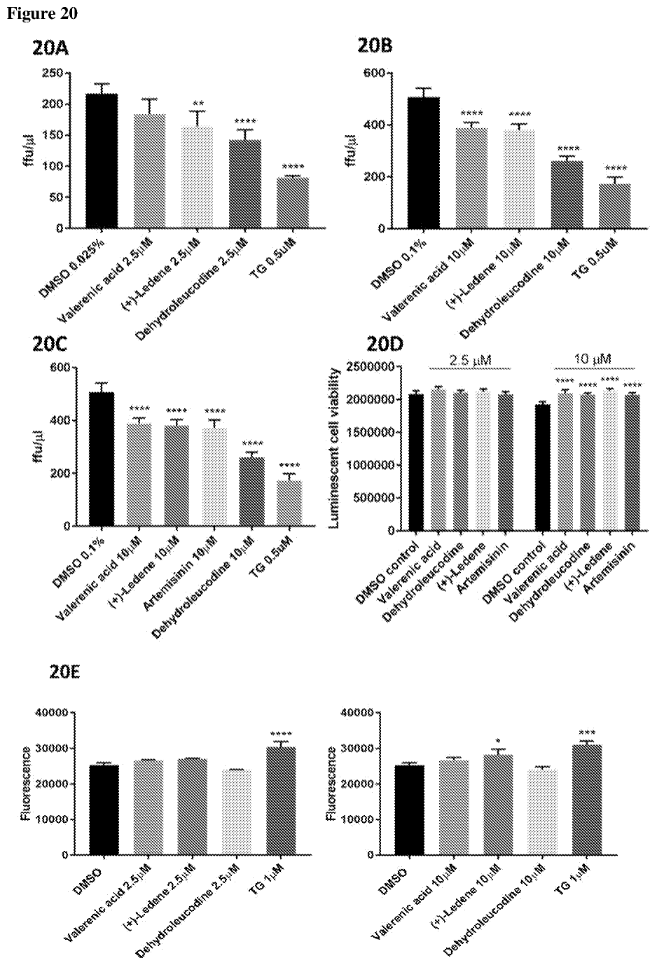

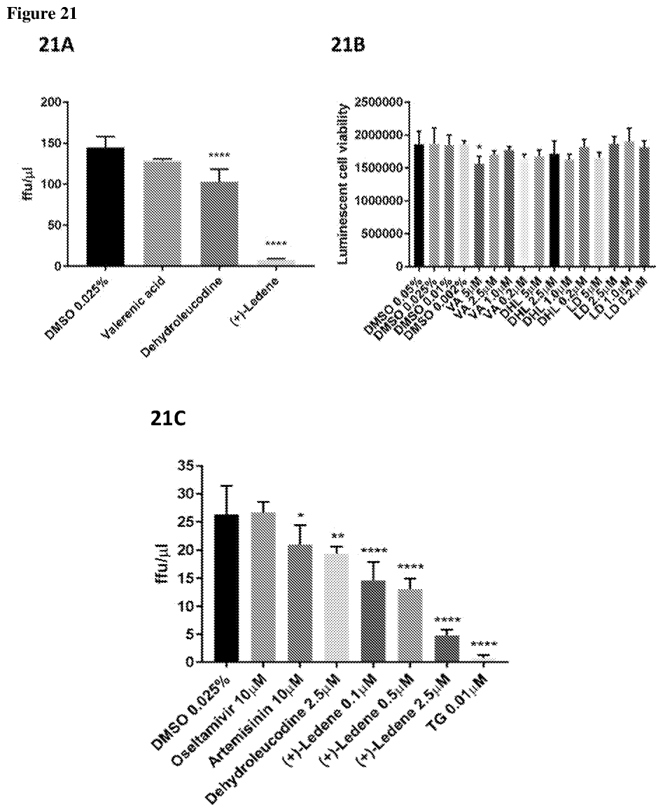

The inventors conducted experiments to compare the effect of various sesquiterpene compounds that show structural similarity to TG in reducing virus production. NPTr and NHBE cells were pre-treated with sesquiterpene compounds (valerenic acid (VA), (+)-ledene (LD), dehydroleucodine (DHL), artemisinin and TG) as indicated for 30 min, rinsed with PBS and infected with USSR H1N1 virus at 0.25 MOI and 0.5 MOI respectively for 24 h. Spun supernatants were used in focus forming assays based on the detection of viral NP in MDCK cells infected for 6 h (FIGS. 20A to 20C, 21A and 21C). NPTr cells were primed with each compound at 2.5 or 10 μM and NHBE cells at 2.5 μM for 30 min, rinsed twice with PBS and cultured overnight for luminescent cell viability assay. Concentrations chosen to prime cells prior to infection had no adverse effect on viability of NPTr (FIG. 20D) and NHBE (FIG. 21B) cells. Results shown in FIG. 20A to 20C and FIGS. 21A and 21C indicate that the tested sesquiterpenes, in particular dehydroleucodine and (+)-ledene, reduced virus production like that of TG. In NHBE cells, pre-treatment with 2.5 μM (+)-ledene resulted in dramatic reduction in progeny virus output (FIGS. 21A and 21C).

The inventors conducted further experiments to determine the effect of the sesquiterpenes and sesquiterpene lactones on extracellular Ca²⁺ influx. Fluo-8 fluorescence intensity was measured in NPTr cells incubated with indicated sesquiterpenes at 2.5 μM or 10 μM for 15 min (FIG. 20E). TG at 1.0 μM was used as a positive control. Significance determined by one way ANOVA in relation to corresponding DMSO control. The data are shown in FIG. 20E.

The inventors have presented compelling evidence that shows SOCE as a potent host innate immune defence against influenza virus infection.

Consistent with this, the inventors showed that the mere facilitation (via over-expression of structural or positive regulatory members of SOCE) or inhibition (via chemical inhibitors or knockdown of STIM1 by siRNA) of SOCE was sufficient to inhibit or promote virus production respectively. Over-expression of STIM1 and Orai isoforms in NPTr cells and myoblasts reduced virus output to a similar extent as the use of TG. Likewise, over-expression of SOCE activators (STIMATE and CRACR2A) in NPTr cells, myoblasts and NHBE cells significantly reduced progeny virus production. Conversely, brief chemical inhibition of Orai channel in NPTr cells, and STIM1 siRNA-knockdown in NHBE cells resulted in raised progeny virus production. Knockdown of STIM1 in NHBE cells reduced the inhibitory effect of TG in virus production, further linking the causal relationship between SOCE and virus inhibition.

A number of sesquiterpenes have been shown by the inventors to exhibit clear antiviral activity (e.g. TG, artemisinin, (+)-ledene and dehydroleucodine). Of these, TG is a SERCA inhibitor hence an activator of CRAC entry via SOCE. Without being bound by theory, the inventors surmise that (+)-ledene, dehydroleucodine and artemisinin also function as facilitators of SOCE upon infection in a manner akin to the antiviral effect seen in the over-expression of SOCE members.

### Example 14

This example demonstrates that sesquiterpene lactones such as thapsigargin are highly selective.

The inventors conducted experiments to determine the selectivity index of TG in primary normal human bronchial epithelial (NHBE) cells and immortalised neonatal pig tracheal epithelial (NPTr) cells.

Cells were primed with a range of TG doses, including DMSO control, for 30 min, washed three times with PBS and following 24 h of incubation, cell viability assay was performed using a CellTiter-Glo Luminescent Cell Viability Assay kit (Promega). CC₅₀ is the concentration of TG used that results in 50% reduction of viability compared with control cells. In a parallel experiment, cells similarly primed with different concentrations of TG were infected with USSR H1N1 virus at 1.0 MOI for NHBE cells or 0.5 MOI for NPTr cells (based on 6 h focus forming assays [ffa] to detect viral NP by immuno-staining) for 24 h and culture media harvested to determine the amount of progeny virus released by ffa detection. IC₅₀ is the dose of TG used that results in 50% reduction of progeny virus in relation to virus output from control cells. Selectivity index (SI) is defined as the ratio CC₅₀/IC₅₀.

The selectivity index in each cell line was determined. The selectivity index in NHBE cells was 8572.89 (cellular cytotoxicity [CC₅₀]=33.52 μM and USSR virus inhibitory concentration [IC₅₀]=0.00391 μM). The selectivity index in NPTr cells was 7952.21 (CC₅₀=56.58 μM and IC₅₀=0.007115 μM). These high selectivity indices indicate a high margin of drug safety.

### Example 15

This example demonstrates that sesquiterpene lactones such as thapsigargin are active against human respiratory syncytial virus (RSV).

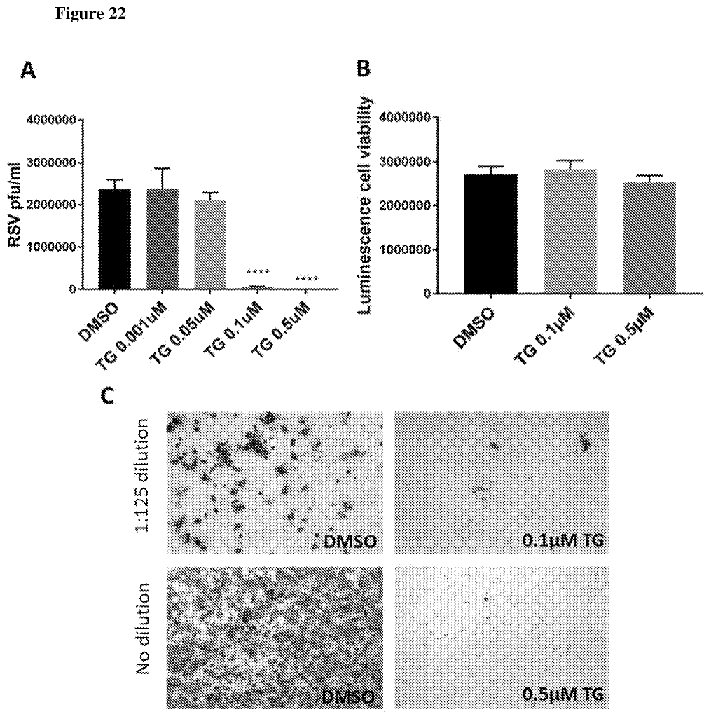

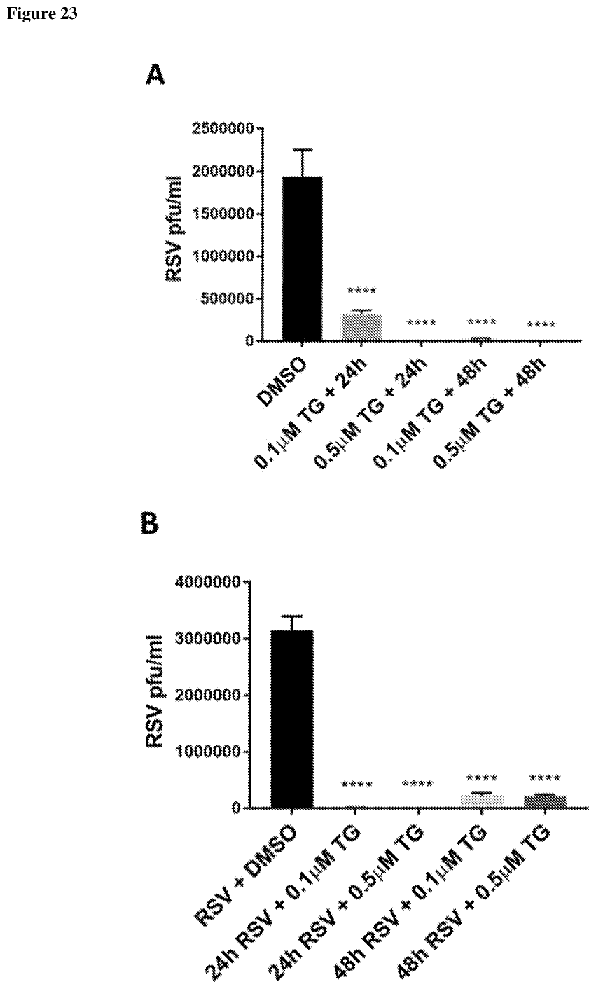

RSV is an enveloped, single negative-strand RNA virus of the Paramyxoviridae family. Human RSV is a major causative agent of respiratory tract infection in children worldwide for which there is still no vaccine available. The inventors conducted experiments to demonstrate that brief 30 min exposure of cells to a non-toxic dose of a sesquiterpene lactones such as thapsigargin is sufficient to effectively block RSV production whether administered before (FIG. 22) or during (FIG. 23) infection.

To demonstrate that TG priming of HEp2 cells at non-toxic doses blocks RSV production, HEp2 cells pre-incubated with indicated concentrations of TG or control DMSO for 30 min were rinsed with serum free media and immediately infected with RSV (A2 strain, ATCC VR-1540) at 0.1 MOI for 3 days. Spun supernatants were collected to infect HEp2 cells for 24 h for immuno-detection of RSV. TG doses used to prime HEp2 cells were non-toxic. 30 min TG treated cells were rinsed, cultured overnight and subjected to cell viability assays (CellTiter-Glo® Luminescent Cell Viability Assay kit, Promega. Results are shown in FIG. 22.

To demonstrate that the TG-activated anti-RSV state in HEp2 cells lasts more than 48 h and is rapidly triggered during infection, HEp2 cells were pre-incubated with TG or control DMSO for 30 min, rinsed with serum free media and further cultured for 24 or 48 h in normal media followed by RSV infection at 0.1 MOI for 3 days. Spun supernatants from infected samples were collected to infect HEp2 cells for 24 h for immuno-detection of RSV. Results are shown in FIG. 23A.

In further experiments, HEp2 cells were first infected with RSV at 0.1 MOI for 24 or 48 h followed by priming with TG or DMSO control for 30 min. Fresh media were used to replace TG containing media of 24 h infected cells; and supernatants collected earlier from 48 h infected cells were used to replace TG containing media of 48 h infected cells. All samples were infected for total period 72 h. Spun supernatants were collected to infect HEp2 cells for 24 h for RSV immuno-detection. DMSO controls were based on combined control supernatants from 24 and 48 h time points. Results are shown in FIG. 23B.

As with influenza virus, TG priming has a sustained anti-viral effect on RSV of over 48 h (FIG. YA) and is rapidly effective in blocking virus production at 48 h post-infection (FIG. YB). The selectivity index of TG over a 3 d infection period in HEp-2 cells is similarly high at 1346.499 (CC₅₀=90 μM and IC₅₀=0.06684 μM).

### REFERENCES

Buckley, B. J., and A. R. Whorton. 1997. Tunicamycin increases intracellular calcium levels in bovine aortic endothelial cells. Am. J. Physiol. 273:C1298-C1305.

Casciano, J. C., N. J. Duchemin, R. J. Lamontagne, L. F. Steel, and M. J. Bouchard. 2017. Hepatitis B virus modulates store-operated calcium entry to enhance viral replication in primary hepatocytes. Plos One 12:e0168328.

Cho, J. A., A. H. Lee, B. Platzer, B. C. S. Cross, B. M. Gardner, H. De Luca, P. Luong, H. P. Harding, L. H. Glimcher, P. Walter, E. Fiebiger, D. Ron, J. C. Kagan, and W. I. Lencer. 2013. The unfolded protein response element IRE1α senses bacterial proteins invading the ER to activate RIG-I and innate immune signaling. Cell Host Microbe 13:558-569.

Chong, A. K., M. S. Peggs, and M. von Itzstein. 1991. Influenza virus sialidase: effect of calcium on steady-state kinetic parameters. Biochim. Biophys. Acta 1077:65-71.

Connor, J. H., and D. S. Lyles. 2005. Inhibition of host and viral translation during vesicular stomatitis virus infection. J. Biol. Chem. 280:13512-13519.

Croisier, H., X. Tan, J. F. Perez-Zoghbi, M. J. Sanderson, J. Sneyd, and B. S. Brook. 2013. Activation of store-operated calcium entry in airway smooth muscle cells: insight from a mathematical model. Plos One 8:e69598.

Cui, R., Y. Wang, L. Wang, G. Li, K. Lan, R. Altmeyer and G. Zou, 2016. Cyclopiazonic acid, an inhibitor of calcium-dependent ATPases with antiviral activity against human respiratory syncytial virus. Antivir. Res. 132:38-45.

Czyz, A., W. Brutkowski, J. Fronk, J. Duszynski, and K. Zablocki. 2009. Tunicamycin desensitizes store-operated Ca²⁺ entry to ATP and mitochondrial potential. Biochem. Biophy. Res. Com. 381:176-180.

Dellis, O., A. Arbabian, B. Papp, M. Rowe, I. Joab, and C. Chomienne. 2011. Epstein-Barr virus latent membrane protein 1 increases calcium influx through store-operated channels in B lymphoid cells. J. Biol. Chem. 286:18583-18592.

Deniaud, A., O. Sharaf el dein, E. Maillier, D. Poncet, G. Kroemer, C. Lemaire, and C. Brenner. 2008. Endoplasmic reticulum stress induces calcium-dependent permeability transition, mitochondrial outer membrane permeabilization and apoptosis. Oncogene 27:285-299.

Denmeade, S. R., C. M. Jakobsen, S. Janssen, S. R. Khan, E. S. Garrett, H. Lilja, S. B. Christensen, and J. T. Isaacs. 2003. Prostate-specific antigen-activated thapsigargin prodrug as targeted therapy for prostate cancer. J. Natl. Cancer 1. 95:990-1000.

Díaz, Y., F. Peña, O. C. Aristimuño, L. Matteo, M. D. Agrela, M. E. Chemello, F. Michelangeli, and M. C. Ruiz. 2012. Dissecting the Ca²⁺ entry pathways induced by rotavirus infection and NSP4-EGFP expression in Cos-7 cells. Virus Res. 167:285-296.

Dombroski, B. A., R. R. Nayak, K. G. Ewens, W. Ankener, V. G. Cheung, and R. S. Spielman. 2010. Gene expression and genetic variation in response to endoplasmic reticulum stress in human cells. Am. J. Hum. Genet. 86:719-729.

Ferrari, M., A. Scalvini, M. N. Losio, A. Corradi, M. Soncini, E. Bignotti, E. Milanesi, P. Ajmone-Marsan, S. Barlati, D. Bellotti, and M. Tonelli. 2003. Establishment and characterization of two new pig cell lines for use in virological diagnostic laboratories. J. Virol. Methods 107:205-212.

Flourakis, M., V. Lehen'kyi, B. Beck, M. Raphaël, M. Vandenberghe, F. V. Abeele, M. Roudbaraki, G. Lepage, B. Mauroy, C. Romanin, R. Skryma, and N. Prevarskaya. 2010. Orai1 contributes to the establishment of an apoptosis-resistant phenotype in prostate cancer cells. Cell Death Dis. 1:e75.

Fujioka, Y., M. Tsuda, A. Nanbo, T. Hattori, J. Sasaki, T. Sasaki, T. Miyazaki, and Y. Ohba. 2013. A Ca²⁺-dependent signalling circuit regulates influenza A virus internalization and infection. Nat. Commun. 4:2763.

Halasz, P., G. Holloway, and B. S. Coulson. 2010. Death mechanisms in epithelial cells following rotavirus infection, exposure to inactivated rotavirus or genome transfection. J. Gen. Virol. 91:2007-2018.

Han, Z., J. J. Madara, A. Herbert, L. I. Prugar, G. Ruthel, J. Lu, Y. Liu, W. Liu, X. Liu, J. E. Wrobel, A. B. Reitz, J. M. Dye, R. N. Harty, and B. D. Freedman. 2015. Calcium regulation of hemorrhagic fever virus budding: mechanistic implications for host-oriented therapeutic intervention. Plos Pathog. 11:e1005220.

Hassan, I. H., M. S. Zhang, L. S. Powers, J. Q. Shao, J. Baltrusaitis, D. T. Rutkowski, K. Legge, and M. M. Monick. 2012. Influenza A viral replication is blocked by inhibition of the inositol-requiring enzyme 1 (IRE1) stress pathway. J. Biol. Chem. 287:4679-4689.

Hogan, P. G., and A. Rao. 2015. Store-operated calcium entry: mechanisms and modulation. Biochem. Biophys. Res. Commun. 460:40-49.

Hrincius, E. R., S. Liedmann, D. Finkelstein, P. Vogel, S. Gansebom, A. E. Samarasinghe, D. You, S. A. Cormier, and J. A. McCullers. 2015. Acute lung injury results from innate sensing of viruses by an ER stress pathway. Cell Rep. 11:1591-1603.

Hyser, J. M., B. Utama, S. E. Crawford, J. R. Broughman, and M. K. Estes. 2013. Activation of the endoplasmic reticulum calcium sensor STIM1 and store-operated calcium entry by rotavirus requires NSP4 viroporin. J. Virol. 87:13579-13588.

Janssens, S., B. Pulendran, and B. N. Lambrecht. 2014. Emerging functions of the unfolded protein response in immunity. Nat. Immunol. 15:910-919.

Jing, J., L. He, A. Sun, A. Quintana, Y. Ding, G. Ma, P. Tan, X. Liang, X. Zheng, L. Chen, X. Shi, S. L. Zhang, L. Zhong, Y. Huang, M. Q. Dong, C. L. Walker, P. G. Hogan, Y. Wang, and Y. Zhou. 2015. Proteomic mapping of ER-PM junctions identifies STIMATE as a regulator of Ca²⁺ influx. Nat. Cell Biol. 17:1339-1347.

Keestra-Gounder, A. M., M. X. Byndloss, N. Seyffert, B. M. Young, A. Chávez-Arroyo, A. Y. Tsai, S. A. Cevallos, M. G. Winter, O. H. Pham, C. R. Tiffany, M. F. de Jong, T. Kerrinnes, R. Ravindran, P. A. Luciw, S. J. McSorley, A. J. Bäumler, and R. M. Tsolis. 2016. NOD1 and NOD2 signalling links ER stress with inflammation. Nature 532:394-397.

Konig, R., S. Stertz, Y. Zhou, A. Inoue, H.-H. Hoffmann, S. Bhattacharyya, J. G. Alamares, D. M. Tscherne, M. B. Ortigoza, Y. Liang, Q. Gao, S. E. Andrews, S. Bandyopadhyay, P. De Jesus, B. P. Tu, L. Pache, C. Shih, A. Orth, G. Bonamy, L. Miraglia, T. Ideker, A. Garcia-Sastre, J. A. T. Young, P. Palese, M. L. Shaw, and S. K. Chanda. 2010. Human host factors required for influenza virus replication. Nature 463:813-817.

Krebs, J., L. B. Agellon, and M. Michalak. 2015. Ca²⁺ homeostasis and endoplasmic reticulum (ER) stress: An integrated view of calcium signaling. Biochem. Biophy. Res. Com. 460:114-121.

Kuchipudi, S. V., S. P. Dunham, R. Nelli, G. A. White, V. J. Coward, M. J. Slomka, I. H. Brown, and K. C. Chang. 2012. Rapid death of duck cells infected with influenza: a potential mechanism for host resistance to H5N1. Immunol. Cell Biol. 90:116-123.

Landera-Bueno, S., Y. Fernández, A. Falcón, J. C. Oliveros, and J. Ortín. 2017. Chemical genomics identifies the PERK-mediated unfolded protein stress response as a cellular target for influenza virus inhibition. mBio 7:e00085-16.

Lencer, W. I., H. DeLuca, M. J. Grey, and J. A. Cho. 2015. Innate immunity at mucosal surfaces: the IRE1-RIDD-RIG-I pathway. Trends Immunol. 36:401-409.

Linford, N. J., and D. M. Dorsa. 2002. 17b-estradiol and the phytoestrogen genistein attenuate neuronal apoptosis induced by the endoplasmic reticulum calcium-ATPase inhibitor thapsigargin. Steroids 67:1029-1040.

Lopez, J. J., L. Albarran, L. J. Gómez, T. Smani, G. M. Salido, and J. A. Rosado. 2016. Molecular modulators of store-operated calcium entry. Biochimica et Biophysica Acta 1853:2037-2043.

Lytton, J., M. Westlin, and M. R. Hanley. 1991. Thapsigargin inhibits the sarcoplasmic or endoplasmic reticulum Ca-ATPase family of calcium pumps. J. Biol. Chem. 266:17067-17071.

Marois, I., A. Cloutier, I. Meunier, H. M. Weingartl, A. M. Cantin, and M. V. Richter. 2014. Inhibition of influenza virus replication by targeting broad host cell pathways. Plos One 9:e110631.

May, V., T. A. Clason, T. R. Buttolph, B. M. Girard, and R. L. Parsons. 2014. Calcium influx, but not intracellular calcium release, supports PACAP-mediated ERK activation in HEK PAC1 receptor cells. J. Mol. Neurosci. 54:342-350.

McNab, F., K. Mayer-Barber, A. Sher, A. Wack, and A. O'Garra. 2015. Type I interferons in infectious disease. Nat. Rev. Immunol. 15:87-103.

Michelangeli, F., F. Liprandi, M. E. Chemello, M. Ciarlet, and M. C. Ruiz. 1995. Selective depletion of stored calcium by thapsigargin blocks rotavirus maturation but not the cytopathic effect. J. Virol. 69:3838-3847.

Oslowski, C. M., and F. Urano. 2011. Measuring ER stress and the unfolded protein response using mammalian tissue culture system. Methods Enzymol. 490:71-92.

Perry, J. W., M. Ahmed, K. O. Chang, N. J. Donato, H. D. Showalter, and C. E. Wobus. 2012. Antiviral activity of a small molecule deubiquitinase inhibitor occurs via induction of the unfolded protein response. Plos Pathog. 8:e1002783.

Quintana, A., V. Rajanikanth, S. Farber-Katz, A. Gudlur, C. Zhang, J. Jing, Y. Zhou, A. Rao, and P. G. Hogan. 2015. TMEM110 regulates the maintenance and remodeling of mammalian ER-plasma membrane junctions competent for STIM-Orai signaling. Proc. Natl. Acad Sci U.S.A. 112:E7083-E7092.

Roberson, E. C., J. E. Tully, A. S. Guala, J. N. Reiss, K. E. Godburn, D. A. Pociask, J. F. Alcorn, D. W. H. Riches, O. Dienz, Y. M. W. Janssen-Heininger, and V. Anathy. 2012. Influenza induces endoplasmic reticulum stress, caspase-12-dependent apoptosis, and cJun N-terminal kinase-mediated transforming growth factor-b release in lung epithelial cells. Am. J. Respir. Cell Mol. Biol. 46:573-581.

Roobol, A., J. Roobol, A. Bastide, J. R. P. Knight, A. E. Willis, and C. M. Smales. 2015. p58^{IPK} is an inhibitor f the eIF2a kinase GCN2 and its localization and expression undepin protein synthesis and ER processing capacity. Biochem. J. 465:213-225.

Sabbah, A., T. H. Chang, R. Harnack, V. Frohlich, K. Tominaga, P. H. Dubei, Y. Xiang, and S. Bose. 2009. Activation of innate immune antiviral responses by Nod2. Nat. Immunol. 10:1073-1080.

Sacchetto, R., I. Bertipaglia, S. Giannetti, L. Cendron, F. Mascarello, E. Damiani, E. Carafoli, and G. Zanotti. 2012. Crystal structure of sarcoplasmic reticulum Ca²⁺-ATPase (SERCA) from bovine muscle. J. Struct. Biol. 178:38-44.

Sebastian, S., L. Goulding, S. Kuchipudi, and K. C. Chang. 2015. Extended 2D myotube culture recapitulates postnatal fibre type plasticity. BMC Cell Biology 16:23.

Seidler, N. W., I. Jona, M. Vegh, and A. Martonosi. 1989. Cyclopiazonic acid is a specific inhibitor of the Ca²⁺-ATPase of sarcoplasmic reticulum. J. Biol. Chem. 264:17816-17823.

Silva, A. M., D. Wang, A. A. Komar, B. A. Castilho, and B. R. G. Williams. 2007. Salicylates trigger protein synthesis inhibition in a protein kinase R-like endoplasmic reticulum kinase-dependent manner. J. Biol. Chem. 282:10164-10171.

Srikanth, S., H. J. Jung, K. D. Kim, P. Souda, J. Whitelegge, and Y. Gwack. 2010. A novel EF-hand protein, CRACR2A, is a cytosolic Ca²⁺ sensor that stabilizes CRAC channels in T cells. Nat. Cell Biol. 12:436-446.

Tsalikis, J., Q. Pan, I. Tattoli, C. Maisonneuve, B. J. Blencowe, D. J. Philpott, and S. E. Girardin. 2016. The transcriptional and splicing landscape of intestinal organoids undergoing nutrient starvation or endoplasmic reticulum stress. BMC Genomics 17:680.

Ueda, M., T. Daidoji, A. Du, C. S. Yang, M. S. Ibrahim, K. Ikuta, and T. Nakaya. 2010. Highly pathogenic H5N1 avian influenza virus induces extracellular calcium influx leading to apoptosis on avian cells. J. Virol. 84:3068-3078.

Uyama, Y., Y. Imaizumi, and M. Watanabe. 1993. Cyclopiazonic acid, an inhibitor of Ca²⁺-ATPase in sarcoplasmic reticulum, increases excitability in ileal smooth muscle. Brit. J. Pharmacol. 110:565-572.

Wang, H., X. Jia, C. Sui, Y. Zhao, Y. Mei, Y. Zheng, and Z. Zhang. 2014. Effects of thapsigargin on the proliferation and survival of human rheumatoid arthritis synovial cells. Sci. World J. 2014:605416.

Yan, W., C. L. Frank, M. J. Korth, B. L. Sopher, I. Novoa, D. Ron, and M. G. Katze. 2002. Control of PERK eIF2a kinase activity by the endoplasmic reticulum stress-induced molecular chaperon P58^{IPK}. Proc. Natl. Acad. Sci. U.S.A. 99:15920-15925.

Yang, B., and M. J. Bouchard. 2012. The hepatitis B virus X protein elevates cytosolic calcium signals by modulating mitochondrial calcium uptake. J. Virol. 86:313-327.

Zhou, Y., T. K. Frey, and J. J. Yang. 2009. Viral calciomics: interplays between Ca²⁺ and virus. Cell Calcium 46:1-17.

Zhu-Mauldin, X., S. A. Marsh, L. Zou, R. B. Marchase, and J. C. Chatham. 2017. Modification of STIM1 by O-linked N-acetylglucosamine (O-GlcNAc) attenuates store-operated calcium entry in neonatal cardiomyocytes. J. Biol. Chem. 287:39094-39106.

1. A Store-Operated Ca²⁺ Entry (SOCE) facilitator for use in the treatment or prevention of viral infection in a subject.

2. An SOCE facilitator for use according to claim 1 wherein said SOCE facilitator is an inhibitor of the sarcoplasmic/endoplasmic reticulum Ca²⁺-ATPase (SERCA) pump.

3. An SOCE facilitator for use according to claim 2 wherein inhibition of the SERCA pump results in endoplasmic reticulum (ER) calcium store depletion and extracellular calcium influx.

4. An SOCE facilitator for use according to any one of claims 1 to 3 wherein said SOCE facilitator inhibits progeny virus production from infected cells.

5. An SOCE facilitator for use according to any one of the preceding claims wherein said SOCE facilitator does not significantly decrease viral RNA expression.

6. An SOCE facilitator for use according to any one of the preceding claims wherein said SOCE facilitator inhibits virus replication in infected cells in the subject.

7. An SOCE facilitator for use according to any one of the preceding claims wherein said SOCE facilitator inhibits virus replication in infected respiratory epithelial cells in the subject.

8. An SOCE facilitator for use according to any one of the preceding claims wherein said SOCE facilitator is a sesquiterpene or a pharmaceutically acceptable salt, derivative or prodrug thereof.

9. An SOCE facilitator for use according to claim 8 wherein said SOCE facilitator is a sesquiterpene lactone or a pharmaceutically acceptable salt, derivative or prodrug thereof.

10. An SOCE facilitator for use according to any one of the preceding claims wherein said SOCE facilitator is a compound of formula (I) or a pharmaceutically acceptable salt, derivative or prodrug thereof, 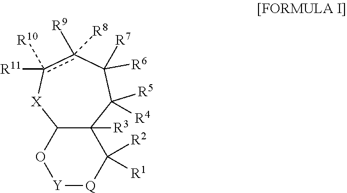
wherein:
X is selected from >C═R^{A}, >CH—R^{A} and —O—;
Y is selected from >C═O and >CH—OR^{Y};
R^{Y} is selected from H, R^{Z}, and —C(O)—R^{Z}; wherein R^{Z} is a C₁₋₂ alkyl group and wherein R^{Z} is unsubstituted or is substituted with —COOH or —C₆H₄COOH;
Q is a bond or is CR¹²R¹³ wherein R¹² and R¹³ are each independently selected from H and methyl;
the moiety 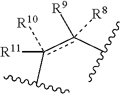
is selected from 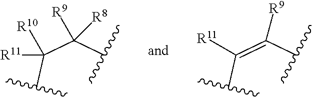
R⁵, R⁶ and R⁷ are each independently selected from H and methyl;
R⁹ is selected from H, —OH, unsubstituted C₁₋₂ alkyl and —OC(O)R^{B};
R⁸ and R¹⁰ if present are each independently selected from H and methyl;
Each RB is independently selected from unsubstituted C₁₋₇ alkyl and unsubstituted C₂₋₇ alkenyl;
and wherein
when X is >C═R^{A} or >CH—R^{A}:
R¹¹ is bonded to R^{A} to form, together with the atoms to which they are attached, a 5-membered carbocyclic group which is substituted by 2 to 4 groups independently selected from —OH, unsubstituted C₁₋₂ alkyl, oxo and —OC(O)R^{B};
R¹ is selected from H and methyl and R² is selected from H, —OH and unsubstituted C₁₋₂ alkyl; or
R¹ and R² together form a methylene moiety such that >CR¹R² is >C═CH₂;
R³ is selected from H, —OH and unsubstituted C₁₋₂ alkyl;
R⁴ is selected from H, —OH, unsubstituted C₁₋₂ alkyl and —OC(O)R^{B}; and
when X is O,
R¹ is selected from H and methyl;
R¹¹ is —O— and R³ is —O— and R¹¹ is bonded to R³ to form a —O—O— linker group;
R⁴ is bonded to R² to form, together with the atoms to which they are attached, a 6-membered carbocyclic group which is substituted by 1 to 3 groups independently selected from —OH and unsubstituted C₁₋₂ alkyl.

11. An SOCE facilitator for use according to claim 10 wherein:
R5 is H; and/or
R6 is H; and/or
R7 is H.

12. An SOCE facilitator for use according to claim 10 or claim 11 wherein X is >C═R^{A} or >CH—R^{A}.

13. An SOCE facilitator for use according to claim 12 wherein R¹¹ is bonded to R^{A} to form, together with the atoms to which they are attached, a 5-membered carbocyclic group which is substituted by (i) two —OC(O)R^{B} groups and by one unsubstituted C₁₋₂ alkyl group or (ii) one oxo group and one unsubstituted C₁₋₂ alkyl group.

14. An SOCE facilitator for use according to claim 13 wherein R¹¹ is bonded to R^{A} to form, together with the atoms to which they are attached, a 5-membered carbocyclic group which is substituted by (i) one methyl group; (ii) one —OC(O)—C₇H₁₅ group and (iii) one —OC(O)—C₄H₇ group.

15. An SOCE facilitator for use according to claim 13 wherein R¹¹ is bonded to R^{A} to form, together with the atoms to which they are attached, a 5-membered carbocyclic group which is substituted by (i) one oxo group and (ii) one methyl group.

16. An SOCE facilitator for use according to any one of claims 10 to 15 wherein said SOCE facilitator is a compound of formula (II) or formula (III) or a pharmaceutically acceptable salt, derivative or prodrug thereof:

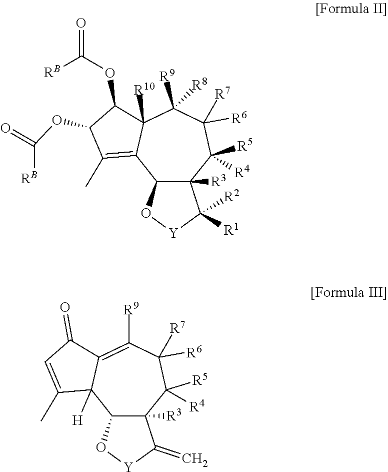

17. An SOCE facilitator for use according to any one of claims 10 to 16 wherein said SOCE facilitator is a compound of formula (IIa) or a pharmaceutically acceptable salt, derivative or prodrug thereof:

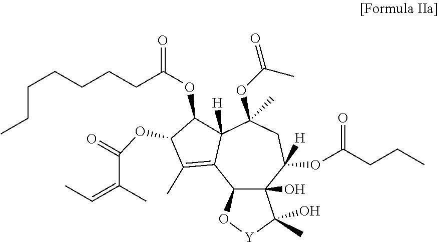

18. An SOCE facilitator for use according to any one of claims 10 to 16 wherein said SOCE facilitator is a compound of formula (IIIa) or a pharmaceutically acceptable salt, derivative or prodrug thereof:

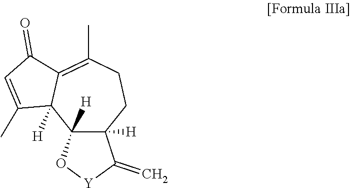

19. An SOCE facilitator for use according to 10 or claim 11 wherein X is —O—.

20. An SOCE facilitator for use according to claim 19 wherein R⁴ is bonded to R² to form, together with the atoms to which they are attached, a 6-membered carbocyclic group which is substituted by 1 unsubstituted C₁₋₂ alkyl group.

21. An SOCE facilitator for use according to any one of claims 10 to 11 or 19 to 20 wherein said SOCE facilitator is a compound of formula (IV) or a pharmaceutically acceptable salt, derivative or prodrug thereof:

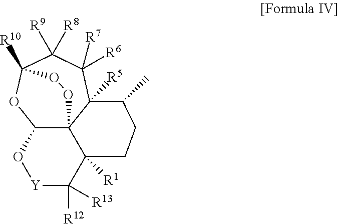

22. An SOCE facilitator for use according to any one of claims 10 to 11 or 19 to 21 wherein said SOCE facilitator is a compound of formula (IVa) or a pharmaceutically acceptable salt, derivative or prodrug thereof:

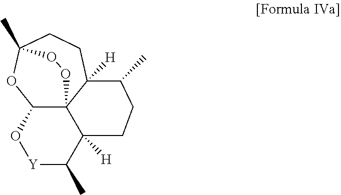

23. An SOCE facilitator for use according to any one of claims 10 to 22 wherein Y is >C═O.

24. An SOCE facilitator for use according to any one of the preceding claims wherein said SOCE facilitator is thapsigargin, artemisinin, (+)-ledene, dehydroleucodine, or valerenic acid; or a pharmaceutically acceptable salt, derivative or prodrug of thapsigargin, artemisinin, (+)-ledene, dehydroleucodine, or valerenic acid.

25. A compound for use in the treatment or prevention of viral infection in a subject in need thereof, wherein said compound is a sesquiterpene or sesquiterpene lactone, wherein preferably said sesquiterpene or sesquiterpene lactone is as defined in any one of claims 10 to 24.

26. A pharmaceutical composition for use in the treatment or prevention of viral infection in a subject in need thereof comprising an SOCE facilitator as defined in any one of claims 1 to 24 or a compound as defined in claim 25 together with at least one pharmaceutically acceptable carrier or diluent.

27. A combination comprising (i) an SOCE facilitator, wherein said SOCE facilitator is preferably as defined in any one of claims 1 to 24; or a compound as defined in claim 25; (ii) an additional antiviral agent; and optionally (iii) at least one pharmaceutically acceptable carrier or diluent.

28. A combination according to claim 27 for use in the treatment or prevention of viral infection in a subject in need thereof.

29. A combination according to claim 27 or combination for use according to claim 28 wherein the antiviral agent is selected from zanamivir, oseltamivir, peramivir, amantadine or rimantadine, or a pharmaceutically acceptable salt of any of the preceding agents.

30. An SOCE facilitator for use according to any one of claims 1 to 24, a compound for use according to claim 25; a pharmaceutical composition for use according to 26 or a combination for use according to claim 28 or claim 29, wherein the viral infection is caused by an RNA virus.

31. An SOCE facilitator for use, compound for use, pharmaceutical composition for use or combination for use according to claim 30 wherein the viral infection is caused by an influenza virus.

32. An SOCE facilitator, compound, pharmaceutical composition or combination for use according to claim 31 wherein the influenza virus is selected from one or more a human influenza A viruses and/or avian influenza A viruses, wherein preferably the influenza virus is selected from one or more of H1N1, H3N2, H5N1, H5N6 and H7N9 viruses.

33. An SOCE facilitator for use, compound for use, pharmaceutical composition for use or combination for use according to claim 30 wherein the viral infection is caused by a virus of the Paramyxoviridae family, preferably respiratory syncytial virus.

34. An SOCE facilitator, compound, pharmaceutical composition or combination for use according to any one of claims 1 to 26 or 28 to 33, wherein said use comprises pulmonary administration of the SOCE facilitator, pharmaceutical composition or combination to the subject.

35. A method of treating or preventing viral infection in a subject, wherein said method comprises administration to the subject of an SOCE facilitator as defined in any one of claims 1 to 24, a compound as defined in claim 25, a pharmaceutical composition as defined in claim 26; or a combination according to any one of claims 27 to 29.

36. An SOCE facilitator as defined in any one of claims 1 to 24, a compound as defined in claim 25; a pharmaceutical composition as defined in claim 26; or a combination according to any one of claims 27 to 29, for use in the manufacture of a medicament for treating or preventing viral infection in a subject.

37. An aerosol formulation comprising an SOCE facilitator or a compound which is a sesquiterpene or sesquiterpene lactone.

38. An aerosol formulation according to claim 37 wherein the SOCE facilitator is as defined in any one of claims 2 to 24, wherein the compound which is a sesquiterpene or sesquiterpene lactone is as defined in claim 25; and/or wherein the SOCE facilitator or compound is present in a pharmaceutical composition or combination as defined in any one of claims 26 to 29.

39. An in vitro method of evaluating the antiviral activity or potential antiviral activity of a compound against a virus, comprising assessing the activity of the compound to facilitate CRAC entry mediated SOCE.

40. A method according to claim 39 further comprising assessing the activity of the compound to reduce infection of cells by the virus.

41. A method according to claim 39 or claim 40 wherein:
i) a fluorescence-based assay for detecting intracellular calcium mobilization is used to assess the activity of the compound to facilitate CRAC entry mediated SOCE; and/or
ii) a hemagglutination assay is used to assess the activity of the compound to reduce virus production from infected cells; and/or
iii) evaluating the antiviral activity or potential antiviral activity of the compound comprises comparing the extent to which the compound (i) facilitates CRAC entry mediated SOCE and optionally (ii) prevents infection of cells by the virus with that of a reference compound, wherein the reference compound is preferably an SOCE facilitator as defined in any one of claims 1 to 24; and/or
iv) the method comprises the high-throughput screening of multiple compounds; and/or
v) the virus is an RNA virus, preferably an influenza virus or a virus of the Paramyxoviridae family, more preferably an influenza A virus.
